(19) United States
(12) Patent Application Publication
(10) Pub. No.: US 2009/0298856 A1
(43) Pub. Date: Dec. 3, 2009

(54) 2,3 Substituted fused bicyclic pyrimidin-4(3H)-ones modulating the function of the vanilliod-1receptor (VR1)

(76) Inventor: Rebecca Elizabeth Brown, Cambridge (GB)

Correspondence Address: MERCK AND CO., INC, P O BOX 2000, RAHWAY, NJ, 07065-0907, US

(21) Appl. No.: 11/919,960

(22) PCT Filed: May 10, 2006
(86) PCT No.: PCT/US06/18119

§ 371 Date: Nov. 17, 2008

(30) Foreign Application Priority Data
May 11, 2005 (GB) 0509574.0
Mar. 1, 2006 (GB) 0604049.7

(51) Int. Cl. A61K 31/519; C07D 473/30; A61K 31/522; C07D 513/04; A61P 19/06; A61P 11/06; A61P 3/04

(52) U.S. Cl. 514/260.1; 544/265; 514/263.3; 544/255

(57) ABSTRACT

The use of a compound of formula (I): for the manufacture of a medicament for the treatment of conditions ameliorated by the modulation of the function of the vanilloid-1 receptor (VR1, also known as TRPV1).

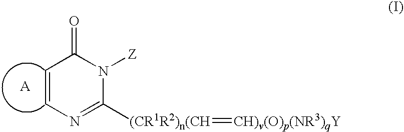

The present invention is concerned with 2,3-substituted fused bicyclic pyrimidin-4(3H)-ones and analogues and derivatives thereof as well as pharmaceutically acceptable salts and prodrugs thereof, which are useful as therapeutic compounds, particularly in the treatment of pain and other conditions ameliorated by the modulation of the function of the vanilloid-1 receptor (VR1, also known as TRPV1).

The pharmacologically active ingredient of chili peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly the VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)-VR1 IC₅₀ of 420 nM. Other sub-micromolar antagonists has also been reported recently (Lee et al, Bioorg. Med. Chem., 9:1713, 2001; Park et al, Bioorg. Med. Chem. Lett., 13:601, 2003; Yoon et al, Bioorg. Med. Chem. Lett., 13:1549, 2003; Lee et al, Bioorg. Med. Chem., 12:3411, 2004; McDonnell et al, Bioorg. Med. Chem. Lett., 14:531, 2004; Ryu et al, Bioorg. Med. Chem. Lett., 14:1751, 2004; Rami et al, Bioorg. Med. Chem. Lett., 14:3631, 2004; Gunthorpe et al, Neuropharmacology 46:133, 2004; Doherty et al, J. Med. Chem., 48:71, 2005), but these reports provide no evidence for in vivo efficacy. A high affinity antagonist has been derived from the potent agonist resiniferatoxin; iodo-resiniferatoxin (Wahl et al., Mol. Pharmacol., 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002).

EP-A-0807633, EP-A-0900799, WO 98/38187 and WO 98/38173 disclose structurally related AMPA receptor antagonists for treating neurodegenerative and CNS-trauma related conditions.

WO-A-9733890 discloses structurally related compounds as pesticides.

U.S. Pat. No. 3,939,161 describes 1,3-dimethyl-1H-pyrazolo[4,3-d]pyrimidine-7(6H)-ones as exhibiting CNS, anti-inflammatory and gastric antisecretory activity.

WO 02/26718 discloses structurally related bicyclic pyrimidin-4-ones as inhibitors of Factor Xₐ, for treating thrombosis conditions.

WO 89/08113 discloses structurally related compounds as immunoregulators and anticancer agents.

BE-B-769844 describes structurally related compounds for disorders such as inflammation.

WO 04/037176 describes structurally related compounds as inhibitors of Factor Xₐ, for treating thromboembolic disorders.

The compounds of the present invention have advantageous properties, such as good in vivo efficacy.

The compounds of the present invention unexpectedly show improved pharmacokinetic properties, such as improved metabolic stability.

We herein describe another novel series of VR1 modulators. These comprise predominantly VR1 antagonists but encompass VR1 partial antagonists and VR1 partial agonists. Such compounds have been shown to be efficacious in animal models of pain.

The use of a compound of formula (I):

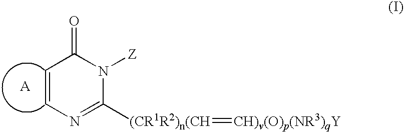

wherein:

A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by one, two or three groups independently chosen from halogen, hydroxy, S(O)ᵣC₁₋₄alkyl, S(O)ᵣNR⁴R⁵, —NR^{x}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆-alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a ring selected from phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

R¹ and R² are independently hydrogen, hydroxy, halogen, C₁₋₆alkyl or haloC₁₋₆alkyl, or R¹ and R² together form an oxo group;

R³ is hydrogen or C₁₋₆alkyl;

each R⁴ and R⁵ is independently hydrogen or C₁₋₆alkyl or R⁴ and R⁵, together with the nitrogen atom to which they are attached, may form a saturated 4-7 membered ring;

R^{x} is hydrogen or C₁₋₆alkyl;

n is zero, one, two, three or four;

v is zero or one;

p and q are both zero or one of p and q is zero and the other is one, provided that when n and v are zero then p and q are both zero;

r is zero, one or two;

Y is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloalkyl ring; a phenyl ring, a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S, the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;

Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by one or more groups independently chosen from halogen, hydroxy, cyano, nitro, NR⁴R⁵ as defined above, S(O)ᵣNR⁴R⁵, —NR^{x}S(O)ᵣC₁₋₆alkyl, S(O)ᵣC₁₋₄alkyl, C₁₋₆alkyl, C₂₋₆alkenyl, C₂₋₆alkynyl, trifluoromethyl, C₁₋₆alkoxy, haloC₁₋₆alkoxy, C₃₋₇cycloalkyl and hydroxyC₁₋₆alkyl;

or a pharmaceutically acceptable salt or tautomer thereof, for the manufacture of a medicament for the treatment or prevention of gout; irritable bowel syndrome; respiratory diseases such as chronic obstructive pulmonary diseases (COPD), chronic bronchitis, cystic fibrosis, asthma and rhinitis, including allergic rhinitis such as seasonal and perennial rhinitis, non-allergic rhinitis and cough; hot flushes; hiccups; obesity; or gastro-oesophageal reflux disease (GERD).

The present invention also provides a method for the treatment or prevention of gout; irritable bowel syndrome; respiratory diseases such as chronic obstructive pulmonary diseases (COPD), chronic bronchitis, cystic fibrosis, asthma and rhinitis, including allergic rhinitis such as seasonal and perennial rhinitis, non-allergic rhinitis and cough; hot flushes; hiccups; obesity; or gastro-oesophageal reflux disease (GERD), which method comprises administration to a patient in need thereof of an effective amount of a compound of formula I or a composition comprising a compound of formula I.

Preferably, the compounds of formula I can be used for the manufacture of a medicament for the treatment or prevention of respiratory diseases such as chronic obstructive pulmonary diseases (COPD), chronic bronchitis, cystic fibrosis, asthma and rhinitis, including allergic rhinitis such as seasonal and perennial rhinitis, non-allergic rhinitis and cough.

In one embodiment the compounds of formula I can be used for the treatment of cough.

In an embodiment of formula I, Y is C₁₋₆alkyl, haloC₁₋₆alkyl, hydroxyC₁₋₆alkyl, aminoC₁₋₆alkyl, carboxyC₁₋₆alkyl; or a C₃₋₇cycloalkyl ring, a phenyl ring, a five-membered heteroaromatic ring containing one, two, three or four heteroatoms independently chosen from O, N and S, at most one heteroatom being O or S, a six-membered heteroaromatic ring containing one, two or three N atoms, a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, or a six-membered saturated ring containing one or two heteroatoms independently chosen from O and N, the ring being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;

v is zero; and

p and q are both zero or one of p and q is zero and the other is one, provided that when n is zero then p and q are both zero.

A is preferably a fused five-membered heteroaromatic ring containing 1, 2 or 3 heteroatoms independently chosen from O, N and S, provided that no more than one O or S atom is present, or a fused six-membered heteroaromatic ring containing 1, 2 or 3 N atoms.

A is more preferably a fused five-membered heteroaromatic ring containing 1, 2 or 3 heteroatoms independently chosen from O, N and S, provided that no more than one O or S atom is present.

In one embodiment A is not a fused pyrazole ring.

In another embodiment A is fused pyridine, thiazole, imidazole or thiophene ring.

In another embodiment A is not a fused thiophene ring.

Preferably A is a fused pyridine, thiazole or imidazole ring. More particularly A is a fused thiazole or imidazole ring, especially an imidazole ring

A is preferably unsubstituted or substituted by halogen, hydroxy, C₃₋₅cycloalkyl, C₁₋₄alkyl, haloC₁₋₄alkyl, C₁₋₄alkoxy, haloC₁₋₄alkoxy or phenyl. More preferably A is unsubstituted or substituted by C₁₋₄alkyl, C₃₋₅cycloalkyl or haloC₁₋₄alkyl. Favourably A is unsubstituted or substituted by C₁₋₄alkyl. More particularly A is unsubstituted or substituted by methyl or ethyl. Further particular substituents on A are propyl, trifluoroethyl, cyclopropyl and difluoroethyl.

In an embodiment A is unsubstituted or substituted by methyl, ethyl, propyl, 2,2,2-trifluoroethyl, cyclopropyl or 2,2-difluoroethyl.

In another embodiment A is unsubstituted or substituted by methyl, ethyl, propyl, 2,2,2-trifluoroethyl or cyclopropyl.

Preferably A is unsubstituted or substituted by one or two groups. More particularly A is monosubstituted. In an embodiment A is unsubstituted or monosubstituted.

When A is substituted by a hydroxy group tautomerism may occur. For example when A is fused imidazole, tautomerism may occur to form an imidazolone.

In one embodiment R¹ and R² are independently hydrogen, hydroxy, halogen, C₁₋₆alkyl or haloC₁₋₆alkyl.

R¹ and R² are independently preferably hydrogen or C₁₋₄alkyl. Most particularly R¹ and R² are both hydrogen.

In an embodiment R¹ and R² are independently selected from hydrogen, methyl and fluorine.

In an embodiment R¹ is hydrogen and R² is hydrogen or C₁₋₆alkyl, preferably hydrogen or methyl.

R³ is preferably hydrogen or C₁₋₂alkyl. R³ may be hydrogen.

In an embodiment R³ is methyl.

In an embodiment each of R⁴ and R⁵ is independently selected from hydrogen and C₁₋₆alkyl.

Preferably n is zero, one, two or three.

In one embodiment n is not two.

In another embodiment n is not zero.

In an embodiment p is zero.

In an embodiment q is zero. In another embodiment q is one.

In one embodiment v is zero.

In an embodiment, when n is zero then v, p and q are zero and when v is one then n is not two.

In another embodiment, n is zero or one and v is zero.

Y is preferably C₁₋₆alkyl, haloC₁₋₆alkyl, hydroxyC₁₋₆alkyl, aminoC₁₋₆alkyl, carboxyC₁₋₆alkyl; or a C₃₋₇cycloalkyl ring, a phenyl ring, a five-membered heteroaromatic ring containing one, two, three or four heteroatoms independently chosen from O, N and S, at most one heteroatom being O or S, a six-membered heteroaromatic ring containing one, two or three N atoms, or a six-membered saturated ring containing one or two heteroatoms independently chosen from O and N, the ring being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵.

In an embodiment Z is optionally substituted phenyl, pyridinyl or thiazolyl.

Z is preferably an optionally substituted phenyl or pyridinyl ring. More particularly Z is an optionally substituted phenyl.

Z is preferably unsubstituted or substituted by one or more substituents independently chosen from cyano, halogen, C₁₋₄alkyl, trifluoromethyl, C₁₋₄alkoxy, haloC₁₋₄alkoxy, amino, C₁₋₄alkylamino and di(C₁₋₄alkyl)amino. A further preferred substituent is amide.

Particular substituents include chlorine, trifluoromethyl, cyano, methyl, fluorine, ethoxy, trifluoromethoxy, bromine, dimethylamino, methoxy and isopropoxy. A further particular substituent is amide.

A favoured substituent is halogen, especially fluorine and chlorine. A further favoured substituent is methyl.

In one embodiment Z is not substituted by trifluoromethyl.

Preferably Z is unsubstituted or substituted by one, two or three groups. More preferably Z is unsubstituted or substituted by one or two groups. Most particularly Z is monosubstituted.

Particular Z groups are fluorophenyl, chlorophenyl, difluorophenyl, (chloro)(fluoro)phenyl, (fluoro)(methyl)phenyl, phenyl, chlorothiazolyl, amidephenyl, cyanophenyl and (chloro)(methyl)phenyl.

Thus, specific Z groups are 4-fluorophenyl and 4-chlorophenyl. Further specific Z groups are 3,4-difluorophenyl, 4-chloro-3-fluorophenyl, 3-fluoro-4-methylphenyl and phenyl. Further specific Z groups are 5-chloro-1,3-thiazol-2-yl, 4-amidephenyl, 4-cyanophenyl and 4-chloro-3-methylphenyl.

The present invention also provides the use of the compounds of formula (I), or a pharmaceutically acceptable salt or tautomer thereof, wherein n, p, q, v, A, R¹, R², R³, Y and Z are as defined above, provided that:

(a) when n is two and v is zero or when n is zero and v is one; and p and q are zero then A is a fused imidazole;

(b) when A is a fused 1,3-dimethyl[4,5]pyrazole ring and Z is optionally substituted phenyl then (CR¹R²)ₙ(CH═CH)ᵥ(O)ₚ(NR³)_{q}Y is not C₁₋₄alkyl, haloC₁₋₄alkyl, morpholinomethyl, piperidinomethyl, methoxymethyl, N-methylpiperazinomethyl or p-chlorophenoxymethyl;

(c) when A is a fused [3,4]thiophene ring then (CR¹R²)ₙ(CH═CH)ᵥ(O)ₚ(NR³)_{q}Y is not C₁₋₇alkyl; and

(d) when A is a fused [3,4]thiophene ring then (CR¹R²)ₙ(CH═CH)ᵥ(O)ₚ(NR³)_{q}Y¹ is not pyridylvinyl;

for the manufacture of a medicament for the treatment or prevention of physiological disorders that may be ameliorated by modulating VR1 activity.

In an embodiment is provided the use of the compounds of formula (I), or a pharmaceutically acceptable salt or tautomer thereof, wherein n, p, q, A, R¹, R², R³, Y and Z are as defined above and v is zero;

provided that:

(a) when n is two then A is a fused imidazole;

(b) when A is a fused 1,3-dimethyl[4,5]pyrazole ring and Z is optionally substituted phenyl then (CR¹R²)ₙ(CH═CH)ᵥ(O)ₚ(NR³)_{q}Y is not C₁₋₄alkyl, haloC₁₋₄alkyl, morpholinomethyl, piperidinomethyl, methoxymethyl, N-methylpiperazinomethyl or p-chlorophenoxymethyl; and

(c) when A is a fused [3,4]thiophene ring then (CR¹R²)ₙ(CH═CH)ᵥ(O)ₚ(NR³)_{q}Y is not C₁₋₇alkyl.

Examples of physiological disorders that may be ameliorated by modulating VR1 activity include pain, such as chronic and acute pain; inflammation disorders; irritable bowel syndrome; urinary incontinence; respiratory diseases; hot flushes; gout; depression, hiccups, obesity and gastro-oesophageal reflux disease (GERD).

The preferences for formula (I) apply mutatis mutandis.

In one embodiment, when n is two and p and q are both zero then Y is C₁₋₆alkyl, haloC₁₋₆alkyl, hydroxyC₁₋₆alkyl, aminoC₁₋₆alkyl, carboxyC₁₋₆alkyl, or an optionally substituted C₃₋₇cycloalkyl ring or six-membered saturated ring containing one or two heteroatoms independently chosen from O and N, pyrid-3-yl, pyrid-4-yl or 3-fluorophenyl.

In one embodiment, when n is two and p and q are both zero then Y is haloC₁₋₆alkyl, an optionally substituted C₃₋₇cycloalkyl ring or 3-fluorophenyl.

In an embodiment when n is two and p and q are both zero then Y is 3-fluorophenyl, cyclohexyl or trifluoromethyl.

In another embodiment when n is two and p and q are both zero then Y is not 3-fluorophenyl.

The present invention also provides novel compounds of formula (IA):

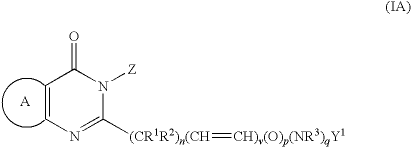

wherein:

A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by one, two or three groups independently chosen from halogen, hydroxy, S(O)ᵣC₁₋₄alkyl, S(O)ᵣNR⁴R⁵, —NR^{x}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl, or R¹ and R² together form an oxo group;

R¹ and R² are independently hydrogen, hydroxy, halogen, C₁₋₆alkyl or haloC₁₋₆alkyl, or R¹ and R² together form an oxo group;

R³ is hydrogen or C₁₋₆alkyl;

each R⁴ and R⁵ is independently hydrogen or C₁₋₆alkyl or R⁴ and R⁵, together with the nitrogen atom to which they are attached, may form a saturated 4-7 membered ring;

R^{x} is hydrogen or C₁₋₆alkyl;

n is zero, one, two, three or four;

v is zero or one;

p and q are both zero or one of p and q is zero and the other is one, provided that when n and v are zero then p and q are both zero;

r is zero, one or two;

Y¹ is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloakyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; a six-membered saturated ring containing one or two heteroatoms independently chosen from O and N; a 8 to 10-membered fused bicyclic partially saturated ring containing a C₅₋₆cycloalkyl ring, a five or a six-membered saturated ring containing one or two heteroatoms independently selected from O, N and S the ring fused to either a phenyl ring, a five-membered heteroaromatic ring as just defined or a six-membered heteroaromatic ring as just defined; or a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, phenyl, morpholino, haloC₁₋₆alkoxy, hydroxyC₁₋₆alkyl, hydroxyC₁₋₆alkoxy and NR⁴R⁵, wherein each of R⁴ and R⁵ are independently selected from hydrogen and C₁₋₆alkyl;

Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by one or more groups independently chosen from halogen, hydroxy, cyano, nitro, NR⁴R⁵ as defined above, S(O)ᵣNR⁴R⁵, —NR^{x}S(O)ᵣC₁₋₆alkyl, S(O)ᵣC₁₋₄alkyl, C₁₋₆alkyl, C₂₋₆alkenyl, C₂₋₆alkynyl, trifluoromethyl, C₁₋₆alkoxy, haloC₁₋₆alkoxy, C₃₋₇cycloalkyl and hydroxyC₁₋₆alkyl;

provided that:

(a) when n is two and v is zero or when n is zero and v is one; and p and q are zero then A is a fused imidazole;

(b) when A is a fused 1,3-dimethyl[4,5]pyrazole ring and Z is an optionally substituted phenyl then (CR¹R²)ₙ(CH═CH)ᵥ(O)ₚ(NR³)_{q}Y¹ is not C₁₋₄alkyl, haloC₁₋₄alkyl, morpholinomethyl, piperidinomethyl, methoxymethyl, N-methylpiperazinomethyl or p-chlorophenoxymethyl;

(c) when A is a fused [3,4]thiophene ring then (CR¹R²)ₙ(CH═CH)ᵥ(O)ₚ(NR³)_{q}Y¹ is not C₁₋₇alkyl;

(d) when A is a fused benzene ring or a fused [3,2]thiophene or [3,2]furan ring; n, p, q and v are zero; Z is an optionally substituted phenyl or optionally substituted pyrid-2-yl ring, and Y¹ is a phenyl, furan, thiophene or pyridine ring; then the Y¹ ring is not substituted by NR⁴R⁵;

(e) when A is a fused [2,3]thiophene ring, Z is optionally substituted phenyl, and p, q and v are zero then Y¹ is not C₁₋₆alkyl or an optionally substituted phenyl, or an optionally substituted 5 or 6 membered heteroaromatic ring or an optionally substituted six-membered saturated ring linked via an N heteroatom;

(f) when A is a fused [3,4]thiophene ring then (CR¹R²)ₙ(CH═CH)ᵥ(O)ₚ(NR³)_{q}Y¹ is not pyridylvinyl;

(g) when A is a fused [3,2]thiophene ring and Z is an optionally substituted phenyl then (CR¹R²)ₙ(CH═CH)ᵥ(O)ₚ(NR³)_{q}Y¹ is not methyl;

(h) when A is a fused benzene ring and Z is an optionally substituted phenyl then (CR¹R²)ₙ(CH═CH)ᵥ(O)ₚ(NR³)_{q}Y¹ is not phenyl or biphenyl; and

(i) when A is a fused [2,3]thiophene ring, n, v, p are zero, q is one and Z is an optionally substituted phenyl then Y¹ is not C₁₋₆alkyl;

or a pharmaceutically acceptable salt or tautomer thereof.

In an embodiment is provided a compound of formula IA, wherein n, A, R¹, R², R³, Z are as defined above;

V is zero;

p and q are both zero or one of p and q is zero and the other is one, provided that when n is zero then p and q are both zero; and

Y¹ is C₁₋₆alkyl, haloC₁₋₆alkyl, hydroxyC₁₋₆alkyl, aminoC₁₋₆alkyl, carboxyC₁₋₆alkyl; or a C₃₋₇cycloalkyl ring; a phenyl ring, a five-membered heteroaromatic ring containing one, two, three or four heteroatoms independently chosen from O, N and S, at most one heteroatom being O or S, a six-membered heteroaromatic ring containing one, two or three N atoms or a six-membered saturated ring containing one or two heteroatoms independently chosen from O and N, the ring being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;

provided that:

(a) when n is two then A is a fused imidazole;

(b) when A is a fused 1,3-dimethyl[4,5]pyrazole ring and Z is an optionally substituted phenyl then (CR¹R²)ₙ(O)ₚ(NR³)_{q}Y¹ is not C₁₋₄alkyl, haloC₁₋₄alkyl, morpholinomethyl, piperidinomethyl, methoxymethyl, N-methylpiperazinomethyl or p-chlorophenoxymethyl;

(c) when A is a fused [3,4]thiophene ring then (CR¹R²)ₙ(O)ₚ(NR³)_{q}Y¹ is not C₁₋₇alkyl;

(d) when A is a fused benzene ring or a fused [3,2]thiophene or [3,2]furan ring; n, p and q are zero; Z is an optionally substituted phenyl or optionally substituted pyrid-2-yl ring; and Y¹ is a phenyl, furan, thiophene or pyridine ring, then the Y¹ ring is not substituted by pyridin-4-yl or NR⁴R⁵; and

(e) when A is a fused [2,3]thiophene ring, n is 1 to 4, Z is optionally substituted phenyl, and p and q are both zero then Y¹ is not C₁₋₆alkyl or an optionally substituted phenyl, or an optionally substituted 5 or 6 membered heteroaromatic ring or an optionally substituted six-membered saturated ring linked via an N heteroatom.

The favoured identities with reference to formula IA are as defined previously for formula I mutatis mutandis.

In one embodiment when n is two and p and q are both zero then Y¹ is C₁₋₆alkyl, haloC₁₋₆alkyl, hydroxyC₁₋₆alkyl, aminoC₁₋₆alkyl, carboxyC₁₋₆alkyl, or an optionally substituted C₃₋₇cycloalkyl ring or six-membered saturated ring containing one or two heteroatoms independently chosen from O and N, or 3-fluorophenyl.

In one embodiment, when n is two and p and q are both zero then Y¹ is haloC₁₋₆alkyl, an optionally substituted C₃₋₇cycloalkyl ring or 3-fluorophenyl.

In an embodiment when n is two and p and q are both zero then Y¹ is 3-fluorophenyl, cyclohexyl or trifluoromethyl

In another embodiment when n is two and p and q are both zero then Y¹ is not 3-fluorophenyl.

In another embodiment Y or Y¹ is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloalkyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; a six-membered saturated ring containing one or two heteroatoms independently chosen from O and N; a 8 to 10-membered fused bicyclic partially saturated ring containing a C₅₋₆cycloalkyl ring, a five or a six-membered saturated ring containing one or two heteroatoms independently selected from O, N and S the ring fused to either a phenyl ring, a five-membered heteroaromatic ring as just defined or a six-membered heteroaromatic ring as just defined; or a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, morpholino, haloC₁₋₆alkoxy, hydroxyC₁₋₆alkyl, hydroxyC₁₋₆alkoxy and NR⁴R⁵, wherein each of R⁴ and R⁵ are independently selected from hydrogen and C₁₋₆alkyl.

Y or Y¹ is preferably C₁₋₆alkyl, haloC₁₋₆alkyl or a C₃₋₇cycloalkyl ring, a phenyl ring, a five-membered heteroaromatic ring containing one, two, three or four heteroatoms independently chosen from O, N and S, at most one heteroatom being O or S, or a six-membered heteroaromatic ring containing one, two or three N atoms, the ring being optionally substituted by one or more groups independently chosen from halogen, C₁₋₄alkyl, hydroxy, C₁₋₄alkoxy, haloC₁₋₄alkyl, phenyl, haloC₁₋₄alkoxy and NR⁴R⁵ where R⁴ and R⁵ are independently C₁₋₄alkyl or, R⁴ and R⁵, together with the nitrogen atom to which they are attached, form a 5 or 6 membered saturated ring.

More particularly, Y or Y¹ is C₁₋₄alkyl, haloC₁₋₄alkyl, a C₃₋₇cycloalkyl ring, a phenyl ring or a five membered heteroaromatic ring containing one, two, three or four heteroatoms independently chosen from O, N and S, providing that no more than one O or S atom is present, the ring being optionally substituted by one or more groups independently selected from halogen, C₁₋₄alkyl and haloC₁₋₄alkyl.

Favourably Y or Y¹ is haloC₁₋₄alkyl, a C₃₋₇cycloalkyl ring or a phenyl ring optionally substituted by one or more groups independently selected from halogen and haloC₁₋₄alkyl.

In an embodiment Y or Y¹ is C₁₋₆alkyl, haloC₁₋₆alkyl, C₂₋₆alkenyl or an optionally substituted ring selected from cyclopropyl, cyclopentyl, cyclohexyl, phenyl, pyridinyl, thiazolyl, oxadiazolyl, tetrahydrobenzothiazolyl, benzoyl, tetrahydronaphthalenyl, oxazolyl, dihydrobenzofuranyl, benzofuranyl, tetrahydrothiopyranyl, dihydroindenyl, benzothiazolyl, dihydrochromenyl, benzoxazolyl, benzofuranyl and benzothienyl.

In an embodiment the optional substituents on the Y or Y¹ ring are selected from halogen, haloC₁₋₄alkyl, C₁₋₄alkyl, morpholino, cyano, phenyl, C₁₋₄alkoxy and oxo.

Particular optional substituents on the Y or Y¹ ring are selected from fluorine, trifluoromethyl, methyl, morpholino, ethyl, cyano, phenyl, chlorine, methoxy, bromine, oxo, isopropyl and tertbutyl.

Preferably when Y or Y¹ is a ring, the ring is unsubstituted or substituted by one, two or three groups. More preferably the ring is unsubstituted or substituted by one or two groups. Most particularly the ring is unsubstituted or monosubstituted.

Favoured substituents on the Y or Y¹ ring are halogen, haloC₁₋₄alkyl and C₁₋₄alkyl. More particular substituents on the Y or Y¹ ring are fluorine, trifluoromethyl and methyl.

Thus, favoured Y or Y¹ groups are trifluoromethyl, cyclohexyl, phenyl, fluorophenyl and trifluoromethylphenyl. Further favoured Y or Y¹ groups are propyl, butyl, cyclopropyl, cyclopentyl, heptafluoropropyl, (trifluoromethyl)pyridinyl, (trifluoromethyl)thiazolyl and methyloxadiazolyl. Further favoured Y or Y¹ groups are (trifluoromethyl)tetrahydrobenzothiazolyl, trifluorophenyl, morpholinopyridinyl, ethylpyridinyl, cyanophenyl, fluorobenzoyl, difluorophenyl, tetraliydronaphthalenyl, ethylthiazolyl, (methyl)(phenyl)oxazolyl, (methyl)(phenyl)thiazolyl, phenylthiazolyl, chloropyridinyl, difluorocyclohexyl, dihydrobenzofuranyl, benzofuranyl, dimethylthiazolyl, chlorophenyl, methylphenyl, methoxyphenyl, bromophenyl, methylphenyl, tetrahydrothiopyranyl, oxocyclohexyl, methyl, (chloro)(fluoro)phenyl, dichlorophenyl, (fluoro)dihydroindenyl, (chloro)difluorophenyl, dihydroindenyl, difluoro(methyl)phenyl, (trifluoromethyl)dihydroindenyl, benzothiazolyl, dihydrochromenyl, benzoxazolyl, vinyl, isopropylcyclohexyl, tertbutylcyclohexyl, (trifluoromethyl)cyclohexyl, chloromethyl, benzofuranyl, benzothienyl and methylbenzothienyl.

Specific Y or Y¹ groups are trifluoromethyl, cyclohexyl, phenyl, 3-fluorophenyl and 3-trifluoromethylphenyl. Further specific Y or Y¹ groups are iso-propyl, tert-butyl, cyclopropyl, cyclopentyl, 1,1,1,2,3,3,3-heptafluoroprop-2-yl, 5-(trifluoromethyl)pyridin-3-yl, 2-(trifluoromethyl)-1,3-thiazol-4-yl and 4-methyl-1,2,5-oxadiazol-3-yl. Further specific Y or Y¹ groups are 2-(trifluoromethyl)-4,5,6,7-tehahydro-1,3-benzothiazol-5-yl, 2,4,6-trifluorophenyl, 6-morpholinopyridin-3-yl, 6-ethylpyridin-3-yl, 3-cyanophenyl, 3-fluorobenzoyl, 2,3-difluorophenyl, 3,5-difluorophenyl, 1,2,3,4-tetrahydronaphthalen-2-yl, 2,4-difluorophenyl, 2-ethyl-1,3-thiazol-4-yl, 5-methyl-2-phenyl-1,3-oxazol-4-yl, 5-methyl-2-phenyl-1,3-thiazol-4-yl, 2-phenyl-1,3-thiazol-4-yl, 2-chloropyridin-4-yl, 2,6-difluorophenyl, 3,4-difluorophenyl, 4,4-difluorocyclohexyl, 2,3-dihydro-1-benzofuran-2-yl, 1-benzofuran-2-yl, 2,3-dihydro-1-benzofuran-3-yl, 2,5-dimethyl-1,3-thiazol-4-yl, 3-chlorophenyl, 3-methylphenyl, 3-methoxyphenyl, 3-bromophenyl, 2-fluorophenyl, 4-fluorophenyl, 2-chlorophenyl, 2,5-difluorophenyl, 3,4,5-trifluorophenyl, 2-trifluoromethylphenyl, 4-methylcyclohexyl, tetrahydro-2H-thiopyran-4-yl, 4-oxocyclohexyl, methyl, 4-chloro-2-fluorophenyl, 2-chloro-4-fluorophenyl, 3-chloro-2-fluorophenyl, 2,4,5-trifluorophenyl, 2,4-dichlorophenyl, 3,4-dichlorophenyl, 2,3,6-trifluorophenyl, 2-chloro-6-fluorophenyl, 2,6-dichlorophenyl, 5-fluoro-2,3-dihydro-1H-inden-1-yl, 2-chloro-3,6-difluorophenyl, 2,3-dihydro-1H-inden-2-yl, 2,3,4-trifluorophenyl, 2,6-difluoro-3-methylphenyl, 3-chloro-2,6-difluorophenyl, 5-(trifluoromethyl)-2,3-dihydro-1H-inden-2-yl, 1,3-benzothiazol-2-yl, 3,4-dihydro-2H-chromen-4-yl, 1,3-benzoxazol-2-yl, vinyl, 4-isopropylcyclohexyl, 4-tertbutylcyclohexyl, 4-(trifluoromethyl)cyclohexyl, chloromethyl, 1-benzofuran-2-yl, 1-benzothien-3-yl and 3-methyl-1-benzothien-2-yl.

In an embodiment Y¹ is not 3-fluorophenyl.

The present invention also provides compounds of formula (IAA):

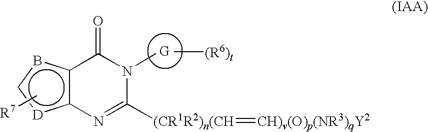

wherein:

B is S and D is C or one of B and D is N and the other N or S;

G is phenyl, pyridine or thiazole;

n is zero, one, two, three or four;

v is zero or one;

p and q are both zero or one of p and q is zero and the other is one, provided that when n and v are zero then p and q are both zero;

t is zero, one or two;

R¹ and R² are independently hydrogen, C₁₋₆alkyl or halogen;

R³ is hydrogen or C₁₋₆alkyl;

R⁶ is cyano, halogen, C₁₋₄alkyl, trifluoromethyl, C₁₋₄alkoxy, haloC₁₋₄alkoxy, amino, C₁₋₄alkylamino, di(C₁₋₆alkyl)amino, amide, C₁₋₄alkylamide or di(C₁₋₆alkyl)amide;

R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;

Y² is C₁₋₆alkyl, C₂₋₆alkenyl, haloC₁₋₆alkyl or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S, the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, or a five or six-membered heteroaromatic ring as just defined, the ring fused to either a five or six membered heteroaromatic ring as just defined; any of which rings being optionally substituted by one or more groups independently chosen from halogen, C₁₋₄alkyl, hydroxy, oxo, C₁₋₄alkoxy, haloC₁₋₄alkyl, cyano, phenyl, haloC₁₋₄alkoxy, morpholino and NR⁴R⁵ where R⁴ and R⁵ are independently hydrogen or C₁₋₄alkyl or, R⁴ and R⁵, together with the nitrogen atom to which they are attached, form a 5 or 6 membered saturated ring;

provided that:

(a) when B is S and D is C; n, p, q and v are zero; G is phenyl or pyrid-2-yl ring; and Y² is a phenyl, furan, thiophene or pyridine ring; then the Y² ring is not substituted by NR⁴R⁵;

(b) when B is S and D is C and G is phenyl then (CR¹R²)ₙ(CH═CH)ᵥ(O)ₚ(NR³)_{q}Y² is not methyl;

(c) when n is two and v is zero or when n is zero and v is one; and p and q are zero then one of B and D is N and the other N or S;

or a pharmaceutically acceptable salt or tautomer thereof.

In an embodiment one of B and D is N and the other N or S.

In an embodiment G is phenyl or 1,3-thiazol-2-yl.

In an embodiment each of R¹ and R² is independently selected from hydrogen, methyl and fluorine.

In an embodiment R⁶ is cyano, halogen, C₁₋₄alkyl or amide. Favoured R⁶ groups include cyano, fluorine, chlorine, methyl and amide.

The present invention also provides compounds of formula (IIA):

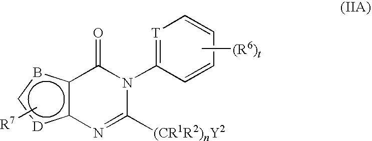

wherein:

one of B and D is N and the other N or S;

T is C or N;

n is zero, one, two, three or four;

t is zero, one or two;

R¹ and R² are independently hydrogen or C₁₋₆alkyl;

R⁶ is cyano, halogen, C₁₋₄alkyl, trifluoromethyl, C₁₋₄alkoxy, haloC₁₋₄alkoxy, amino, C₁₋₄alkylamino or di(C₁₋₆alkyl)amino;

R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;

Y² is C₁₋₆alkyl, haloC₁₋₆alkyl or a C₃₋₇cycloalkyl ring, a phenyl ring, a five-membered heteroaromatic ring containing one, two, three or four heteroatoms independently chosen from O, N and S, at most one heteroatom being O or S, a six-membered heteroaromatic ring containing one, two or three N atoms, an 8 to 10-membered fused bicyclic partially saturated ring containing a C₅₋₆cycloalkyl ring fused to either a phenyl ring, a five-membered heteroaromatic ring as just defined or a six-membered heteroaromatic ring as just defined, the ring being optionally substituted by one or more groups independently chosen from halogen, C₁₋₄alkyl, hydroxy, C₁₋₄alkoxy, haloC₁₋₄alkyl, phenyl, haloC₁₋₄alkoxy and NR⁴R⁵ where R⁴ and R⁵ are independently C₁₋₄alkyl or, R⁴ and R⁵, together with the nitrogen atom to which they are attached, form a 5 or 6 membered saturated ring;

or a pharmaceutically acceptable salt or tautomer thereof.

The favoured identities with reference to formula IAA and IIA are as defined previously for formulae I and IA and described below for formula IB mutatis mutandis.

In one embodiment is provided compounds of formula (IB):

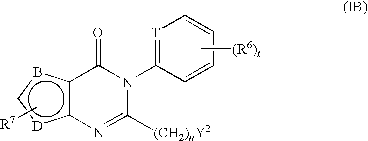

wherein:

one of B and D is N and the other N or S;

T is C or N;

n is zero, one, two, or three;

t is zero, one or two;

R⁶ is cyano, halogen, C₁₋₄alkyl, trifluoromethyl, C₁₋₄alkoxy, haloC₁₋₄alkoxy, amino, C₁₋₄alkylamino or di(C₁₋₆alkyl)amino;

R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;

Y² is C₁₋₆alkyl, C₂₋₆alkenyl, haloC₁₋₆alkyl or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S, the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, or a five or six-membered heteroaromatic ring as just defined, the ring fused to either a five or six membered heteroaromatic ring as just defined; any of which rings being optionally substituted by one or more groups independently chosen from halogen, C₁₋₄alkyl, hydroxy, oxo, C₁₋₄alkoxy, haloC₁₋₄alkyl, cyano, phenyl, haloC₁₋₄alkoxy, morpholino and NR⁴R⁵ where R⁴ and R⁵ are independently hydrogen or C₁₋₄alkyl or, R⁴ and R⁵, together with the nitrogen atom to which they are attached, form a 5 or 6 membered saturated ring;

or a pharmaceutically acceptable salt or tautomer thereof.

In an embodiment of formula BB, Y² is C₁₋₆alkyl, haloC₁₋₆alkyl or a C₃₋₇cycloalkyl ring, a phenyl ring, a five-membered heteroaromatic ring containing one, two, three or four heteroatoms independently chosen from O, N and S, at most one heteroatom being O or S, a six-membered heteroaromatic ring containing one, two or three N atoms, an 8 to 10-membered fused bicyclic partially saturated ring containing a C₅₋₆cycloalkyl ring fused to either a phenyl ring, a five-membered heteroaromatic ring as just defined or a six-membered heteroaromatic ring as just defined, the ring being optionally substituted by one or more groups independently chosen from halogen, C₁₋₄alkyl, hydroxy, C₁₋₄alkoxy, haloC₁₋₄alkyl, phenyl, haloC₁₋₄alkoxy and NR⁴R⁵ where R⁴ and R⁵ are independently C₁₋₄alkyl or, R⁴ and R⁵, together with the nitrogen atom to which they are attached, form a 5 or 6 membered saturated ring.

In one embodiment B and D are both N.

In another embodiment B is N and D is S.

In an embodiment when B and D are both N then R⁷ is attached at D.

When one of B and D is N and the other S then R⁷ is attached at the carbon ring atom.

In an embodiment T is C.

In one embodiment n is not two.

In another embodiment n is not zero.

Preferably t is one or two. In one embodiment t is one.

In an embodiment R¹ and R² are both hydrogen. In another embodiment R¹ is hydrogen and R² is C₁₋₆alkyl, preferably methyl.

In an embodiment each of R⁴ and R⁵ is independently selected from hydrogen and C₁₋₆alkyl. In another embodiment each of R⁴ and R⁵ is independently selected from C₁₋₆alkyl.

In an embodiment R⁶ is substituted at the para position.

Preferably, R⁶ is cyano, C₁₋₆alkyl or halogen. More particularly R⁶ is C₁₋₆alkyl or halogen, especially methyl, chlorine or fluorine.

In an embodiment, each R⁶ is independently cyano or halogen. More particularly R⁶ is halogen, especially chlorine or fluorine.

Preferably R⁷ is hydrogen, C₁₋₄alkyl, C₃₋₅cycloalkyl or halo C₁₋₄alkyl. More particularly R⁷ is hydrogen, methyl, ethyl, propyl, 2,2,2-trifluoroethyl or cyclopropyl. A further particular group is 2,2-difluoroethyl.

In an embodiment R⁷ is C₁₋₄alkyl, C₃₋₅cycloalkyl or haloC₁₋₄alkyl. More particularly R⁷ is C₁₋₄alkyl, especially methyl or ethyl.

In an embodiment Y² is C₁₋₆alkyl, haloC₁₋₆alkyl, C₂₋₆alkenyl or an optionally substituted ring selected from cyclopropyl, cylopentyl, cyclohexyl, phenyl, pyridinyl, thiazolyl, oxadiazolyl, tetrahydrobenzothiazolyl, benzoyl, tetrahydronaphthalenyl, oxazolyl, dihydrobenzofuranyl, benzofuranyl, tetrahydrothiopyranyl, dihydroindenyl, benzothiazolyl, dihydrochromenyl, benzoxazolyl, benzofuranyl and benzothienyl.

Preferably, Y² is C₁₋₆alkyl, haloC₁₋₆alkyl, a ring selected from cyclopropyl, cyclopentyl, cyclohexyl, phenyl, pyridinyl, thiazolyl, oxadiazolyl and tetrahydrobenzothiazolyl, the ring being optionally substituted by one or more groups independently chosen from halogen, C₁₋₄alkyl, hydroxy, C₁₋₄alkoxy, haloC₁₋₄alkyl, phenyl, haloC₁₋₄alkoxy and NR⁴R⁵.

In an embodiment the optional substituents on the Y² ring are selected from halogen, haloC₁₋₄alkyl, C₁₋₄alkyl, morpholino, cyano, phenyl, C₁₋₄alkoxy and oxo.

Particular optional substituents on the Y² ring are selected from fluorine, trifluoromethyl, methyl, morpholino, ethyl, cyano, phenyl, chlorine, methoxy, bromine, oxo, isopropyl and tertbutyl.

Favoured substituents on the Y² ring are halogen, haloC₁₋₄alkyl and C₁₋₄alkyl. More particular substituents on the Y² ring are fluorine, trifluoromethyl and methyl.

Preferably Y² is haloC₁₋₄alkyl, a C₃₋₇cycloalkyl ring or a phenyl ring optionally substituted by one or more groups independently selected from halogen and haloC₁₋₄alkyl.

Preferably when Y² is a ring, the ring is unsubstituted or substituted by one, two or three groups. More preferably the ring is unsubstituted or substituted by one or two groups. Most particularly the ring is unsubstituted or monosubstituted.

Thus, favoured Y² groups are trifluoromethyl, cyclohexyl, phenyl, fluorophenyl, trifluoromethylphenyl, propyl, butyl, heptafluoropropyl, cyclopropyl, cyclopentyl, (trifluoromethyl)pyridinyl, (trifluoromethyl)thiazolyl, methyloxadiazolyl and (trifluoromethyl)tetrahydrobenzothiazolyl. Further favoured Y² groups are trifluorophenyl, morpholinopyridinyl, ethylpyridinyl, cyanophenyl, fluorobenzoyl, difluorophenyl, tetrahydronaphthalenyl, ethylthiazolyl, (methyl)(phenyl)oxazolyl, (methyl)(phenyl)thiazolyl, phenylthiazolyl, chloropyridinyl, difluorocyclohexyl, dihydrobenzofuranyl, benzofuranyl, dimethylthiazolyl, chlorophenyl, methylphenyl, methoxyphenyl, bromophenyl, methylphenyl, tetrahydrothiopyranyl, oxocyclohexyl, methyl, (chloro)(fluoro)phenyl, dichlorophenyl, (fluoro)dihydroindenyl, (chloro)difluorophenyl, dihydroindenyl, difluoro(methyl)phenyl, (trifluoromethyl)dihydroindenyl, benzothiazolyl, dihydrochromenyl, benzoxazolyl, vinyl, isopropylcyclohexyl, tertbutylcyclohexyl, (trifluoromethyl)cyclohexyl, chloromethyl, benzofuranyl, benzothienyl and methylbenzothienyl.

Specific Y² groups are trifluoromethyl, cyclohexyl, phenyl, 3-fluorophenyl, 3-trifluoromethylphenyl, iso-propyl, tert-butyl, 1,1,1,2,3,3,3-heptafluoroprop-2-yl, cyclopropyl, cyclopentyl, 5-trifluoromethyl)pyridin-3-yl, 2-trifluoromethyl)-1,3-thiazol-4-yl, 4-methyl-1,2,5-oxadiazol-3-yl and 2-(trifluoromethyl)-4,5,6,7-tetrahydro-1,3-benzothiazol-5-yl. Further specific Y² groups are 2,4,6-trifluorophenyl, 6-morpholinopyridin-3-yl, 6-ethylpyridin-3-yl, 3-cyanophenyl, 3-fluorobenzoyl, 2,3-difluorophenyl, 3,5-difluorophenyl, 1,2,3,4-tetrahydronaphthalen-2-yl, 2,4-difluorophenyl, 2-ethyl-1,3-thiazol-4-yl, 5-methyl-2-phenyl-1,3-oxazol-4-yl, 5-methyl-2-phenyl-1,3-thiazol-4-yl, 2-phenyl-1,3-thiazol-4-yl, 2-chloropyridin-4-yl, 2,6-difluorophenyl, 3,4-difluorophenyl, 4,4-difluorocyclohexyl, 2,3-dihydro-1-benzofuran-2-yl, 1-benzofuran-2-yl, 2,3-dihydro-1-benzofuran-3-yl, 2,5-dimethyl-1,3-thiazol-4-yl, 3-chlorophenyl, 3-methylphenyl, 3-methoxyphenyl, 3-bromophenyl, 2-fluorophenyl, 4-fluorophenyl, 2-chlorophenyl, 2,5-difluorophenyl, 3,4,5-trifluorophenyl, 2-trifluoromethylphenyl, 4-methylcyclohexyl, tetrahydro-2H-thiopyran-4-yl, 4-oxocyclohexyl, methyl, 4-chloro-2-fluorophenyl, 2-chloro-4-fluorophenyl, 3-chloro-2-fluorophenyl, 2,4,5-trifluorophenyl, 2,4-dichlorophenyl, 3,4-dichlorophenyl, 2,3,6-trifluorophenyl, 2-chloro-6-fluorophenyl, 2,6-dichlorophenyl, 5-fluoro-2,3-dihydro-1H-inden-1-yl, 2-chloro-3,6-difluorophenyl, 2,3-dihydro-1H, inden-2-yl, 2,3,4-trifluorophenyl, 2,6-difluoro-3-methylphenyl, 3-chloro-2,6-difluorophenyl, 5-(trifluoromethyl)-2,3-dihydro-1H-inden-2-yl, 1,3-benzothiazol-2-yl, 3,4-dihydro-2H-chromen-4-yl, 1,3-benzoxazol-2-yl, vinyl, 4-isopropylcyclohexyl, 4-tertbutylcyclohexyl, 4-(trifluoromethyl)cyclohexyl, chloromethyl, 1-benzofuran-2-yl, 1-benzothien-3-yl and 3-methyl-1-benzothien-2-yl.

In one embodiment when n is two then Y² is C₁₋₆alkyl, haloC₁₋₆alkyl or an optionally substituted C₃₋₇cycloalkyl ring or 3-fluorophenyl;

In an embodiment Y² is not 3-fluorophenyl.

The present invention also provides compounds of formula (IIB):

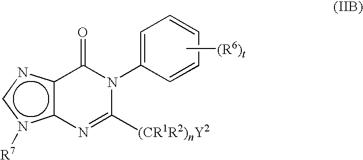

wherein n, t, R¹, R², R⁶, R⁷ and Y² are as defined above;
or a pharmaceutically acceptable salt or tautomer thereof.

The present invention also provides compounds of formula (IC):

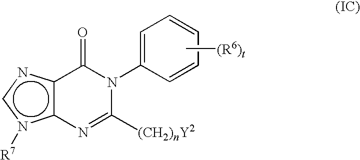

wherein n, t, R⁶, R⁷ and Y² are as defined above;
or a pharmaceutically acceptable salt or tautomer thereof.

The favoured identities with reference to formulae IIB and IC are as defined previously for formulae I and IB mutatis mutandis.

Particular embodiments of the invention include:
1-(4-chlorophenyl)-2-[2-(3-fluorophenyl)ethyl]-9-methyl-1,9-dihydro-6H-purin-6-one;
1-(4-chlorophenyl)-9-ethyl-2-[3-(trifluoromethyl)phenyl]-1,9-dihydro-6H-purin-6-one;
1-(4-chlorophenyl)-9-methyl-2-(4,4,4-trifluorobutyl-1,9-dihydro-6H-purin-6-one;
1-(4-fluorophenyl)-9-methyl-2-(4,4,4-trifluorobutyl)-1,9-dihydro-6H-purin-6-one;
1-(4-chlorophenyl)-9-methyl-2-(3,3,3-trifluoropropyl)-1,9-dihydro-6H-purin-6-one;
1-(4-chlorophenyl)-2-(2-cyclohexylethyl)-9-methyl-1,9-dihydro-6H-purin-6-one;
1-(4-chlorophenyl)-9-methyl-2-(3-phenylpropyl)-1,9-dihydro-6H-purin-6-one;
9-methyl-1-phenyl-2-(4,4,4-trifluorobutyl)-1,9-dihydro-6H-purin-6-one;
6-(4-chlorophenyl)-5-(4,4,4-trifluorobutyl)[1,3]thiazolo[5,4-d]pyrimidin-7(6H)-one;
1-(4-chlorophenyl)-9-methyl-2-(5,5,5-trifluoropentyl)-1,9-dihydro-6H-purin-6-one;
1-(4-chlorophenyl)-9-methyl-2-[3,4,4,4-tetrafluoro-3-(fluoromethyl)butyl]-1,9-dihydro-6H-purin-6-one;
1-(4-chlorophenyl)-9-methyl-2-(3-phenoxypropyl)-1,9-dihydro-6H-purin-6-one;
1-(4-chlorophenyl)-9-methyl-2-[2-trifluoromethyl)-1,3-thiazol-4-yl]-1,9-dihydro-6H-purin-6-one;
1-(4-chlorophenyl)-9-ethyl-2-(4,4,4-trifluorobutyl)-1,9-dihydro-6H-purin-6-one;
1-(4-chlorophenyl)-9-cyclopropyl-2-(4,4,4-trifluorobut-yl)-1,9-dihydro-6H-purin-6-one;
9-ethyl-1-(4-fluorophenyl)-2-(4,4,4-trifluorobutyl)-1,9-dihydro-6H-purin-6-one;
1-(3,4-difluorophenyl)-9-ethyl-2-(4,4,4-trifluorobutyl)-1,9-dihydro-6H-purin-6-one;
1-(4-chloro-3-fluorophenyl)-9-ethyl-2-(4,4,4-trifluorobutyl)-1,9-dihydro-6H-purin-6-one;
1-(4-chlorophenyl)-2-(4,4,4-trifluorobutyl)-9-(2,2,2-trifluoroethyl)-1,9-dihydro-6H-purin-6-one;
1-(4-fluorophenyl)-9-propyl-2-(4,4,4-trifluorobutyl)-1,9-dihydro-6H-purin-6-one;
1-(4-chlorophenyl)-9-propyl-2-(4,4,4-trifluorobutyl)-1,9-dihydro-6H-purin-6-one;
9-ethyl-1-(3-fluoro-4-methylphenyl)-2-(4,4,4-trifluorobutyl)-1,9-dihydro-6H-purin-6-one;
1-(4-chlorophenyl)-9-methyl-2-(4-methylpentyl)-1,9-dihydro-6H-purin-6-one;
1-(4-chlorophenyl)-2-cyclohexyl-9-methyl-1,9-dihydro-6H-purin-6-one;
1-(4-chlorophenyl)-9-methyl-2-(3-methylbutyl)-1,9-dihydro-6H-purin-6-one;
1-(4-chlorophenyl)-9-ethyl-2-(3-methylbutyl)-1,9-dihydro-6H-purin-6-one;
9-ethyl-1-(4-fluorophenyl)-2-(3-methylbutyl)-1,9-dihydro-6H-purin-6-one;
2-tert-butyl-1-(4-chlorophenyl)-9-methyl-1,9-dihydro-6H-purin-6-one;
1-(4-chlorophenyl)-9-methyl-2-{2-[5-(trifluoromethyl)pyridin-3-yl]ethyl}-1,9-dihydro-6H-purin-6-one;
1-(4-chlorophenyl)-9-methyl-2-[2-(trifluoromethyl)-4,5,6,7-tetrahydro-1,3-benzothiazol-5-yl]-1,9-dihydro-6H-purin-6-one;
1-(4-chlorophenyl)-2-(2-cyclopentylethyl)-9-methyl-1,9-dihydro-6H-purin-6-one;
1-(4-chlorophenyl)-2-(2-cyclopropylethyl)-9-ethyl-1,9-dihydro-6H-purin-6-one;
1-(4-chlorophenyl)-9-methyl-2-[(4-methyl-1,2,5-oxadiazol-3-yl)methyl]-1,9-dihydro-6H-purin-6-one;
1-(4-chlorophenyl)-9-methyl-2-(1-methyl-2-[trifluoromethyl)-1,3-thiazol-4-yl]ethyl)-1,9-dihydro-6H-purin-6-one;
1-(4-chlorophenyl)-9-methyl-2-(4,4,4-trifluoro-1-methylbutyl)-1,9-dihydro-6H-purin-6-one;
1-(5-chloro-1,3-thiazol-2-yl)-9-methyl-2-(2,4,6-trifluorobenzyl)-1,9-dihydro-6H-purin-6-one;
1-(4-chlorophenyl)-9-methyl-2-(2,4,6-trifluorobenzyl)-1,9-dihydro-6H-purin-6-one;
1-(4-chlorophenyl)-9-ethyl-2-((6-morpholinopyridin-3-yl)methyl)-1H-purin-6(9H)-one;
1-(4-chlorophenyl)-9-ethyl-2-((6-ethylpyridin-3-yl)methyl)-1H-purin-6(9H)-one;
3-((1-(4-chlorophenyl)-9-ethyl-6-oxo-6,9-dihydro-1H-purin-2-yl)methyl)benzonitrile
1-(4-chlorophenyl)-9-ethyl-2-(3-fluorobenzoyl)-1H-purin-6(9H)-one;
1-(4-chlorophenyl)-9-ethyl-2-(1-(3-(trifluoromethyl)phenyl)ethyl)-1H-purin-6(9H)-one;
1-(4-chlorophenyl)-9-ethyl-2-[3-(trifluoromethyl)benzyl]-1,9-dihydro-6H-purin-6-one;
1-(4-chlorophenyl)-9-cyclopropyl-2-(2,4,6-trifluorobenzyl)-1,9-dihydro-6H-purin-6-one
1-(4-chlorophenyl)-9-cyclopropyl-2-(2,3-difluorobenzyl)-1,9-dihydro-6H-purin-6-one;
1-(4-chlorophenyl)-9-cyclopropyl-2-(2,4-difluorobenzyl)-1,9-dihydro-6H-purin-6-one;
1-(4-chlorophenyl)-9-cyclopropyl-2-(3,5-difluorobenzyl)-1,9-dihydro-6H-purin-6-one;
1-(4-chlorophenyl)-9-cyclopropyl-2-(5,5,5-trifluoropentyl)-1,9-dihydro-6H-purin-6-one;
1-(4-chlorophenyl)-9-cyclopropyl-2-(1,2,3,4-tetrahydronaphthalen-2-yl)-1,9-dihydro-6H-purin-6-one;
6-(4-chlorophenyl)-5-(2,4,6-trifluorobenzyl)[1,3]thiazolo[5,4-d]pyrimidin-7(6H)-one;
6-(4-chlorophenyl)-5-(2,3-difluorobenzyl)-[1,3]thiazolo[5,4-d]pyrimidin-7(6)-one;
6-(4-chlorophenyl)-5-(3,5-difluorobenzyl)[1,3]thiazolo[5,4-d]pyrimidin-7(6H)-one;
6-(4-chlorophenyl)-5-(2,4-difluorobenzyl)[1,3]thiazolo[5,4-d]pyrimidin-7(6H)-one;
6-(4-chlorophenyl)-5-(5,5,5-trifluoropentyl)[1,3]thiazolo[5,4-d]pyrimidin-7(6H)-one;
6-(4-chlorophenyl)-5-(4,4,4-trifluoro-3-methylbutyl)[1,3]thiazolo[5,4-d]pyrimidin-7(6H)-one;
1-(4-chlorophenyl)-9-ethyl-2-(5,5,5-trifluoropentyl)-1,9-dihydro-6H-purin-6-one;
1-(4-chlorophenyl)-9-ethyl-2-(2-ethyl-1,3-thiazol-4-yl)methyl]-1,9-dihydro-6H-purin-6-one;
6-(4-chlorophenyl)-5-(4,4,4-trifluoro-2-methylbutyl)[1,3]thiazolo[5,4-d]pyrimidin-7(6H)-one;
6-(4-chlorophenyl)-5-(1,2,3,4-tetrahydronaphthalen-2-yl)[1,3]thiazolo[5,4-d]pyrimidin-7(6H)-one;
6-(4-chlorophenyl)-5-(cyclohexylmethyl)[1,3]thiazolo[5,4-d]pyrimidin-7-(6H)-one;
1-(4-chlorophenyl)-2-(cyclohexylmethyl)-9-cyclopropyl-1H-purin-6(9H)-one;
1-(4-chlorophenyl)-9-ethyl-2-[(5-methyl-2-phenyl-1,3-oxazol-4-yl)methyl]-1,9-dihydro-6H-purin-6-one;
1-(4-chlorophenyl)-9-ethyl-2-[(5-methyl-2-phenyl-1,3-thiazol-4-yl)methyl]-1,9-dihydro-6H-purin-6-one;
1-(4-chlorophenyl)-9-ethyl-2-[(2-phenyl-1,3-thiazol-4-yl)methyl]-1,9-dihydro-6H-purin-6-one;
1-(4-chlorophenyl)-2-(2-chloropyridin-4-yl)-9-ethyl-1,9-dihydro-6H-purin-6-one;
1-(4-chlorophenyl)-9-ethyl-2-[fluoro(3-fluorophenyl)methyl]-1,9-dihydro-6H-purin-6-one;
1-(4-chlorophenyl)-2-[(2,6-difluorophenyl)(fluoro)methyl]-9-ethyl-1,9-dihydro-6H-purin-6-one;
1-(4-chlorophenyl)-2-[(3,4-difluorophenyl)(fluoro)methyl]-9-ethyl-1,9-dihydro-6H-purin-6-one;
1-(4-chlorophenyl)-2-[(3,4-difluorophenyl)(difluoro)methyl]-9-ethyl-1,9-dihydro-6H-purin-6-one;
1-(4-chlorophenyl)-2-[(4,4-difluorocyclohexyl)methyl]-9-ethyl-1,9-dihydro-6H-purin-6-one;
1-(4-chlorophenyl)-2-[(4,4-difluorocyclohexyl)methyl]-9-methyl-1,9-dihydro-6H-purin-6-one;
1-(4-chlorophenyl)-9-ethyl-2-[1-(5-methyl-2-phenyl-1,3-oxazol-4-yl)ethyl]-1,9-dihydro-6H-purin-6-one;
1-(4-chlorophenyl)-2-[1-(3,5-difluorophenyl)ethyl]-9-ethyl-1,9-dihydro-6H-purin-6-one;
1-(4-chlorophenyl)-2-[1-(2,6-difluorophenyl)ethyl]-9-ethyl-1,9-dihydro-6H-purin-6-one;
1-(4-chlorophenyl)-2-[2-(2,3-dihydro-1-benzofuran-2-yl)ethyl]-9-ethyl-1,9-dihydro-6H-purin-6-one;
2-[(E)-2-(1-benzofuran-2-yl)vinyl]-1-(4-chlorophenyl)-9-methyl-1,9-dihydro-6H-purin-6-one;
1-(4-chlorophenyl)-2-(2,3-dihydro-1-benzofuran-3-ylmethyl)-9-ethyl-1,9-dihydro-6H-purin-6-one;
1-(4-chlorophenyl)-2-(2,3-dihydro-1-benzofuran-3-ylmethyl)-9-methyl-1,9-dihydro-6H-purin-6-one;
1-(4-chlorophenyl)-2-[(2,5-dimethyl-1,3-thiazol-4-yl)methyl]-9-ethyl-1,9-dihydro-6H-purin-6-one;
2-(3-chlorobenzyl)-1-(4-chlorophenyl)-9-ethyl-1,9-dihydro-6H-purin-6-one;
1-(4-chlorophenyl)-9-ethyl-2-(3-methylbenzyl)-1,9-dihydro-6H-purin-6-one;
1-(4-chlorophenyl)-9-ethyl-2-(3-methoxybenzyl)-1,9-dihydro-6H-purin-6-one;
2-(3-bromobenzyl)-1-(4-chlorophenyl)-9-ethyl-1,9-dihydro-6H-purin-6-one;
1-(4-chlorophenyl)-9-ethyl-2-(1-phenylethyl)-1,9-dihydro-6H-purin-6-one;
1-(4-chlorophenyl)-9-ethyl-2-(2-fluorobenzyl)-1,9-dihydro-6H-purin-6-one;
1-(4-chlorophenyl)-9-ethyl-2-(4-fluorobenzyl)-1,9-dihydro-6H-purin-6-one;
1-(4-chlorophenyl)-9-ethyl-2-[1-(3-fluorophenyl)ethyl]-1,9-dihydro-6H-purin-6-one;
1-(4-chlorophenyl)-2-(2,3-difluorobenzyl)-9-ethyl-1,9-dihydro-6H-purin-6-one;
1-(4-chlorophenyl)-2-(3,4-difluorobenzyl)-9-ethyl-1,9-dihydro-6H-purin-6-one;
1-(4-chlorophenyl)-2-(3,5-difluorobenzyl)-9-ethyl-1,9-dihydro-6H-purin-6-one;
2-benzyl-1-(4-chlorophenyl)-9-ethyl-1,9-dihydro-6H-purin-6-one;
2-(2-chlorobenzyl)-1-(4-chlorophenyl)-9-ethyl-1,9-dihydro-6H-purin-6-one;
1-(4-chlorophenyl)-2-(2,6-difluorobenzyl)-9-ethyl-1,9-dihydro-6H-purin-6-one;
1-(4-chlorophenyl)-2-(2,4-difluorobenzyl)-9-ethyl-1,9-dihydro-6H-purin-6-one;
1-(4-chlorophenyl)-2-(2,5-difluorobenzyl)-9-ethyl-1,9-dihydro-6H-purin-6-one;
1-(4-chlorophenyl)-9-ethyl-2-(3,4,5-trifluorobenzyl)-1,9-dihydro-6H-purin-6-one;
1-(4-chlorophenyl)-9-ethyl-2-[2-(trifluoromethyl)benzyl]-1,9-dihydro-6H-purin-6-one;
1-(4-chlorophenyl)-9-ethyl-2-(2,4,6-trifluorobenzyl)-1,9-dihydro-6H-purin-6-one;
1-(4-chlorophenyl)-2-(cyclohexylmethyl)-9-ethyl-1,9-dihydro-6H-purin-6-one;
1-(4-chlorophenyl)-9-ethyl-2-(3,3,3-trifluoro-2-methylpropyl)-1,9-dihydro-6H-purin-6-one;
1-(4-chlorophenyl)-9-ethyl-2-(1,2,3,4-tetrahydronaphthalen-2-yl)-1,9-dihydro-6H-purin-6-one;
1-(4-chlorophenyl)-9-ethyl-2-[(4-methylcyclohexyl)methyl]-1,9-dihydro-6H-purin-6-one;
1-(4-chlorophenyl)-9-ethyl-2-tetrahydro-2H-thiopyran-4-ylmethyl)-1,9-dihydro-6H-purin-6-one;
1-(4-chlorophenyl)-9-methyl-2-(3,3,3-trifluoro-2-methylpropyl)-1,9-dihydro-6H-purin-6-one;
1-(4-chlorophenyl)-9-ethyl-2-[(4-oxocyclohexyl)methyl]-1,9-dihydro-6H-purin-6-one;
1-(4-chlorophenyl)-9-ethyl-2-(4,4,4-trifluoro-2-methylbutyl-1,9-dihydro-6H-purin-6-one;
1-(4-chlorophenyl)-9-methyl-2-(4,4,4-trifluoro-2-methylbutyl)-1,9-dihydro-6H-purin-6-one;
1-(4-chlorophenyl)-9-methyl-2-(1,2,3,4-tetrahydronaphthalen-2-yl)-1,9-dihydro-6H-purin-6-one;
1-(4-fluorophenyl)-9-methyl-2-(1,2,3,4-tetrahydronaphthalen-2-yl)-1,9-dihydro-6H-purin-6-one;
9-ethyl-1-(4-fluorophenyl)-2-(4,4,4-trifluoro-2-methylbutyl)-1,9-dihydro-6H-purin-6-one;
1-(4-fluorophenyl)-9-methyl-2-(4,4,4-trifluoro-2-methylbutyl)-1,9-dihydro-6H-purin-6-one;
1-(4-chlorophenyl)-9-cyclopropyl-2-(4,4,4-trifluoro-2-methylbutyl)-1,9-dihydro-6H-purin-6-one;
1-(4-chlorophenyl)-9-ethyl-2-(4,4,4-trifluoro-3-methylbutyl)-1,9-dihydro-6H-purin-6-one;
1-(4-chlorophenyl)-9-methyl-2-(4,4,4-trifluoro-3-methylbutyl)-1,9-dihydro-6H-purin-6-one;
1-(4-chlorophenyl)-9-ethyl-2-(5,5,5-trifluoro-4-methylpentyl)-1,9-dihydro-6H-purin-6-one;
1-(4-chlorophenyl)-9-methyl-2-(5,5,5-trifluoro-4-methylpentyl)-1,9-dihydro-6H-purin-6-one;
9-ethyl-1-(4-fluorophenyl)-2-(4,4,4-trifluoro-3-methylbutyl)-1,9-dihydro-6H-purin-6-one;
1-(4-fluorophenyl)-9-methyl-2-(4,4,4-trifluoro-3-methylbutyl)-1,9-dihydro-6H-purin-6-one;
1-(4-chlorophenyl)-2-(2,6-difluorobenzyl)-9-(2,2,2-trifluoroethyl)-1,9-dihydro-6H-purin-6-one;
1-(4-chlorophenyl)-2-(2,6-difluorobenzyl)-9-(2,2-difluoroethyl)-1,9-dihydro-6H-purin-6-one;
1-(4-chlorophenyl)-9-(2,2,2-trifluoroethyl)-2-(4,4,4-trifluoro-2-methylbutyl)-1,9-dihydro-6H-purin-6-one;
1-(4-chlorophenyl)-9-(2,2-difluoroethyl)-2-(4,4,4-trifluoro-2-methylbutyl)-1,9-dihydro-6H-purin-6-one;
1-(4-chlorophenyl)-9-ethyl-2-(4-methylpentyl)-1,9-dihydro-6H-purin-6-one;
9-ethyl-1-(4-fluorophenyl)-2-(5,5,5-trifluoro-4-methylpentyl)-1,9-dihydro-6H-purin-6-one;
1-(4-fluorophenyl)-9-methyl-2-(5,5,5-trifluoro-4-methylpentyl)-1,9-dihydro-6H-purin-6-one;
1-(4-chlorophenyl)-2-(2,2-dimethylpropyl)-9-ethyl-1,9-dihydro-6H-purin-6-one;
1-(4-chlorophenyl)-2-(2,2-dimethylpropyl)-9-methyl-1,9-dihydro-6H-purin-6-one;
9-ethyl-1-(4-fluorophenyl)-2-(4-methylpentyl)-1,9-dihydro-6H-purin-6-one;
1-(4-fluorophenyl)-9-methyl-2-(4-methylpentyl)-1,9-dihydro-6H-purin-6-one;
1-(4-chlorophenyl)-2-(2,2-dimethylbutyl)-9-ethyl-1,9-dihydro-6H-purin-6-one;
2-(2,6-difluorobenzyl)-9-ethyl-1-(4-fluorophenyl)-1,9-dihydro-6H-purin-6-one;
9-ethyl-1-(4-fluorophenyl)-2-(2,4,6-trifluorobenzyl)-1,9-dihydro-6H-purin-6-one;
2-(2,4-difluorobenzyl)-9-ethyl-1-(4-fluorophenyl)-1,9-dihydro-6H-purin-6-one;
2-(2,5-difluorobenzyl)-9-ethyl-1-(4-fluorophenyl) 1,9-dihydro-6H-purin-6-one;
9-ethyl-1-(4-fluorophenyl)-2-[2-(trifluoromethyl)benzyl]-1,9-dihydro-6H-purin-6-one;
9-ethyl-2-(2-fluorobenzyl)-1-(4-fluorophenyl)-1,9-dihydro-6H-purin-6-one;
2-(3,5-difluorobenzyl)-9-ethyl-1-(4-fluorophenyl)-1,9-dihydro-6H-purin-6-one;
2-(2-chlorobenzyl)-9-ethyl-1-(4-fluorophenyl)-1,9-dihydro-6H-purin-6-one;
9-ethyl-1-(4-fluorophenyl)-2-[3-(trifluoromethyl)benzyl]-1,9-dihydro-6H-purin-6-one;
1-(4-chlorophenyl)-2-(2,4-difluorobenzyl)-9-methyl-1,9-dihydro-6H-purin-6-one;
1-(4-chlorophenyl)-2-(2,5-difluorobenzyl)-9-methyl-1,9-dihydro-6H-purin-6-one;
1-(4-chlorophenyl)-2-(2,3-difluorobenzyl)-9-methyl-1,9-dihydro-6H-purin-6-one;
1-(4-chlorophenyl)-2-(2,6-difluorobenzyl)-9-methyl-1,9-dihydro-6H-purin-6-one;
1-(4-chlorophenyl)-2-(3,5-difluorobenzyl)-9-methyl-1,9-dihydro-6H-purin-6-one;
2-(2,3-difluorobenzyl)-9-ethyl-1-(4-fluorophenyl)-1,9-dihydro-6H-purin-6-one;
9-ethyl-1-(4-fluorophenyl)-2-{1-[3-(trifluoromethyl)phenyl]ethyl}-1,9-dihydro-6H-purin-6-one;
9-ethyl-1-(4-fluorophenyl)-2-[1-(2,4,6-trifluorophenyl)ethyl]-1,9-dihydro-6H-purin-6-one;
1-(4-chlorophenyl)-2-(2-fluorobenzyl)-9-methyl-1,9-dihydro-6H-purin-6-one;
2-(2-chlorobenzyl)-1-(4-chlorophenyl)-9-methyl-1,9-dihydro-6H-purin-6-one;
2-(4-chloro-2-fluorobenzyl)-1-(4-chlorophenyl)-9-methyl-1,9-dihydro-6H-purin-6-one;
2-(2-chloro-4-fluorobenzyl)-1-(4-chlorophenyl)-9-methyl-1,9-dihydro-6H-purin-6-one;
2-(3-chloro-2-fluorobenzyl)-1-(4-chlorophenyl)-9-methyl-1,9-dihydro-6H-purin-6-one;
9-ethyl-1-(4-fluorophenyl)-2-(2,4,5-trifluorobenzyl)-1,9-dihydro-6H-purin-6-one;
2-(2-chloro-4-fluorobenzyl)-9-ethyl-1-(4-fluorophenyl)-1,9-dihydro-6H-purin-6-one;
2-(3-chloro-2-fluorobenzyl)-9-ethyl-1-(4-fluorophenyl)-1,9-dihydro-6H-purin-6-one;
2-(4-chloro-2-fluorobenzyl)-9-ethyl-1-(4-fluorophenyl)-1,9-dihydro-6H-purin-6-one;
2-(2,4-dichlorobenzyl)-9-ethyl-1-(4-fluorophenyl)-1,9-dihydro-6H-purin-6-one;
2-(3,4-dichlorobenzyl)-9-ethyl-1-(4-fluorophenyl)-1,9-dihydro-6H-purin-6-one;
9-ethyl-1-(4-fluorophenyl)-2-(2,3,6-trifluorobenzyl)-1,9-dihydro-6H-purin-6-one;
1-(4-chlorophenyl)-2-(2,4-dichlorobenzyl)-9-methyl-1,9-dihydro-6H-purin-6-one;
1-(4-chlorophenyl)-2-(3,4-dichlorobenzyl)-9-methyl-1,9-dihydro-6H-purin-6-one;
1-(4-chlorophenyl)-9-methyl-2-(2,3,6-trifluorobenzyl)-1,9-dihydro-6H-purin-6-one;
2-(2-chloro-4-fluorobenzyl)-1-(4-fluorophenyl)-9-methyl-1,9-dihydro-6H-purin-6-one;
2-(4-chloro-2-fluorobenzyl)-1-(4-fluorophenyl)-9-methyl-1,9-dihydro-6H-purin-6-one;
2-(2,4-difluorobenzyl)-1-(4-fluorophenyl)-9-methyl-1,9-dihydro-6H-purin-6-one;
2-(2,3-difluorobenzyl)-1-(4-fluorophenyl)-9-methyl-1,9-dihydro-6H-purin-6-one;
2-(4-chloro-2-fluorobenzyl)-3-(4-chlorophenyl)thieno[3,2-d]pyrimidin-4(3H)-one;
2-(2-chloro-6-fluorobenzyl)-1-(4-chlorophenyl)-9-methyl-1,9-dihydro-6H-purin-6-one;
1-(4-chlorophenyl)-2-(2,6-dichlorobenzyl)-9-methyl-1,9-dihydro-6H-purin-6-one;
1-(4-chlorophenyl)-9-ethyl-2-(5-fluoro-2,3-dihydro-1H-inden-1-yl)-1,9-dihydro-6H-purin-6-one;
2-(2-chloro fluorobenzyl)-3-(4-chlorophenyl)thieno[3,2-d]pyrimidin-4(3H)-one;
2-(4-chloro-2-fluorobenzyl)-3-(4-chlorophenyl)-7-methylthieno[3,2-d]pyrimidin-4(3H)-one;
2-(4-chloro-2-fluorobenzyl)-1-(3,4-difluorophenyl)-9-methyl-1,9-dihydro-6H-purin-6-one;
2-(2-chloro-4-fluorobenzyl)-1-(3,4-difluorophenyl)-9-methyl-1,9-dihydro-6H-purin-6-one;
2-(2,4-dichlorobenzyl-1-(3,4-difluorophenyl)-9-methyl-1,9-dihydro-6H-purin-6-one;
4-[2-(2-chloro-4-fluorobenzyl)-9-methyl-6-oxo-6,9-dihydro-1H-purin-1-yl]benzamide;
2-(2,6-dichlorobenzyl)-1-(4-fluorophenyl)-9-methyl-1,9-dihydro-6H-purin-6-one;
2-(2,4-dichlorobenzyl)-1-(4-fluorophenyl)-9-methyl-1,9-dihydro-6H-purin-6-one;
4-[2-(2-chloro-4-fluorobenzyl)-9-methyl-6-oxo-6,9-dihydro-1H-purin-1-yl]benzonitrile;
2-(2,3-difluorobenzyl)-1-(3,4-difluorophenyl)-9-methyl-1,9-dihydro-6H-purin-6-one;
2-(2,4-difluorobenzyl)-1-(3,4-difluorophenyl)-9-methyl-1,9-dihydro-6H-purin-6-one;
1-(3,4-difluorophenyl)-9-methyl-2-(2,4,6-trifluorobenzyl)-1,9-dihydro-6H-purin-6-one;
4-[2-(4-chloro-2-fluorobenzyl)-9-methyl-6-oxo-6,9-dihydro-1H-purin-1-yl]benzonitrile;
4-[2-(2,4-difluorobenzyl)-9-methyl-6-oxo-6,9-dihydro-1H-purin-1-yl]benzonitrile;
4-[2-(2,3-difluorobenzyl)-9-methyl-6-oxo-6,9-dihydro-1H-purin-1-yl]benzonitrile;
1-(4-fluorophenyl)-9-methyl-2-(2,4,6-trifluorobenzyl)-1,9-dihydro-6H-purin-6-one;
1-(4-fluorophenyl)-9-methyl-2-(2,3,6-trifluorobenzyl)-1,9-dihydro-6H-purin-6-one;
2-(2-chloro-3,6-difluorobenzyl)-1-(4-fluorophenyl)-9-methyl-1,9-dihydro-6H-purin-6-one;
3-(4-chlorophenyl)-7-methyl-2-(2,4,6-trifluorobenzyl)thieno[3,2-d]pyrimidin-4(3H)-one;
2-(2-chloro-3,6-difluorobenzyl)-1-(4-chlorophenyl)-9-methyl-1,9-dihydro-6H-purin-6-one;
2-(2-chloro-3,6-difluorobenzyl)-1-(3,4-difluorophenyl)-9-methyl-1,9-dihydro-6H-purin-6-one;
2-(2,3-dihydro-1H-inden-2-yl)-1-(4-fluorophenyl)-9-methyl-1,9-dihydro-6H-purin-6-one;
1-(4-chlorophenyl)-9-methyl-2-(2,3,4-trifluorobenzyl) 1,9-dihydro-6H-purin-6-one;
1-(4-chlorophenyl)-2-(2,6-difluoro-3-methylbenzyl)-9-methyl-1,9-dihydro-6H-purin-6-one;
2-(3-chloro-2,6-difluorobenzyl)-1-(4-chlorophenyl)-9-methyl-1,9-dihydro-6H-purin-6-one;
2-(2,6-difluoro-3-methylbenzyl)-1-(4-fluorophenyl)-9-methyl-1,9-dihydro-6H-purin-6-one;
2-(3-chloro-2,6-difluorobenzyl)-1-(4-fluorophenyl)-9-methyl-1,9-dihydro-6H-purin-6-one;
4-[9-methyl-6-oxo-2-(2,4,6-trifluorobenzyl)-6,9-dihydro-1H-purin-1-yl]benzonitrile;
1-(4-fluorophenyl)-2-isopropyl-9-methyl-1,9-dihydro-6H-purin-6-one;
1-(4-chlorophenyl)-2-isopropyl-9-methyl-1,9-dihydro-6H-purin-6-one;
1-(4-chlorophenyl)-9-ethyl-2-isopropyl-1,9-dihydro-6H-purin-6-one;
1-(4-fluorophenyl)-9-methyl-2-(2,3,4-trifluorobenzyl) 1,9-dihydro-6H-purin-6-one;
1-(4-chloro-3-fluorophenyl)-2-(2,4-difluorobenzyl)-9-methyl-1,9-dihydro-6H-purin-6-one;
1-(4-chloro-3-fluorophenyl)-9-methyl-2-(2,4,6-trifluorobenzyl)-1,9-dihydro-6H-purin-6-one;
1-(4-chloro-3-fluorophenyl)-9-methyl-2-(2,3,6-trifluorobenzyl)-1,9-dihydro-6H-purin-6-one;
1-(4-chloro-3-fluorophenyl)-9-methyl-2-(2,3,4-trifluorobenzyl)-1,9-dihydro-6H-purin-6-one;
2-(4-chloro-2-fluorobenzyl)-1-(4-chloro-3-fluorophenyl)-9-methyl-1,9-dihydro-6H-purin-6-one;
2-(2-chloro-4-fluorobenzyl)-1-(4-chloro-3-fluorophenyl)-9-methyl-1,9-dihydro-6H-purin-6-one;
1-(4-chloro-3-methylphenyl)-2-(2,4-difluorobenzyl)-9-methyl-1,9-dihydro-6H-purin-6-one;
2-(4-chloro-2-fluorobenzyl)-1-(4-chloro-3-methylphenyl)-9-methyl-1,9-dihydro-6H-purin-6-one;
2-(2-chloro-4-fluorobenzyl)-1-(4-chloro-3-methylphenyl)-9-methyl-1,9-dihydro-6H-purin-6-one;
1-(4-chloro-3-methylphenyl)-9-methyl-2-(2,4,6-trifluorobenzyl)-1,9-dihydro-6H-purin-6-one;
1-(4-chloro-3-methylphenyl)-9-methyl-2-(2,3,4-trifluorobenzyl) 1,9-dihydro-6H-purin-6-one;
1-(4-chloro-3-methylphenyl)-9-methyl-2-(2,3,6-trifluorobenzyl)-1,9-dihydro-6H-purin-6-one;
3-(4-chlorophenyl)-7-methyl-2-(4,4,4-trifluorobutyl)thieno[3,2-d]pyrimidin-4(3H)-one;
1-(4-chlorophenyl)-2-(2,3-dihydro-1H-inden-2-yl)-9-ethyl-1,9-dihydro-6H-purin-6-one;
1-(4-chlorophenyl)-2-(2,3-dihydro-1H-inden-2-yl)-9-methyl-1,9-dihydro-6H-purin-6-one;
1-(3,4-difluorophenyl)-2-(2,3-dihydro-1H-inden-2-yl)-9-methyl-1,9-dihydro-6H-purin-6-one;
1-(4-fluorophenyl)-9-methyl-2-[5-trifluoromethyl)-2,3-dihydro-1H-inden-2-yl]-1,9-dihydro-6H-purin-6-one;
2-[2-(1,3-benzothiazol-2-yl)ethyl]-1-(4-chlorophenyl)-9-ethyl-1,9-dihydro-6H-purin-6-one;
2-[2-(1,3-benzothiazol-2-yl)ethyl]-1-(4-chlorophenyl)-9-methyl-1,9-dihydro-6H-purin-6-one;
1-(4-chlorophenyl)-9-methyl-2-[5-trifluoromethyl)-2,3-dihydro-1H-inden-2-yl]-1,9-dihydro-6H-purin-6-one;
1-(4-chlorophenyl)-9-ethyl-2-[5-(trifluoromethyl)-2,3-dihydro-1H-inden-2-yl]-1,9-dihydro-6H-purin-6-one;
2-[2-(1,3-benzoxazol-2-yl)ethyl]-1-(4-chlorophenyl)-9-ethyl-1,9-dihydro-6H-purin-6-one;
1-(4-chlorophenyl)-2-(3,4-dihydro-2H-chromen-4-yl)-9-ethyl-1,9-dihydro-6H-purin-6-one;
2-[2-(1,3-benzoxazol-2-yl)ethyl]-1-(4-chlorophenyl)-9-methyl-1,9-dihydro-6H-purin-6-one;
1-(4-chlorophenyl)-2-[2-(dimethylamino)ethyl]-9-ethyl-1,9-dihydro-6H-purin-6-one;
1-(4-chlorophenyl)-2-(3,4-dihydro-2H-chromen-4-yl)-9-methyl-1,9-dihydro-6H-purin-6-one;
1-(4-chlorophenyl)-9-ethyl-2-vinyl-1,9-dihydro-6H-purin-6-one;
1-(4-chlorophenyl)-2-(2,2-dimethylbutyl)-9-methyl-1,9-dihydro-6H-purin-6-one;
1-(4-chlorophenyl)-2-(3,3-dimethylbutyl)-9-ethyl-1,9-dihydro-6H-purin-6-one;
1-(4-chlorophenyl)-2-(3,3-dimethylbutyl)-9-methyl-1,9-dihydro-6H-purin-6-one;
1-(4-chlorophenyl)-9-(2,2-difluoroethyl)-2-(3,3,3-trifluoro-2-methylpropyl)-1,9-dihydro-6H-purin-6-one;
1-(4-chlorophenyl)-9-(2,2-difluoroethyl)-2-(4,4,4-trifluorobutyl)-1,9-dihydro-6H-purin-6-one;
1-(4-chlorophenyl)-9-(2,2-difluoroethyl)-2-(4,4,4-trifluoro-3-methylbutyl)-1,9-dihydro-6H-purin-6-one;
1-(4-chlorophenyl)-9-(2,2-difluoroethyl)-2-(5,5,5-trifluoropentyl)-1,9-dihydro-6H-purin-6-One;
1-(4-chlorophenyl)-9-(2,2,2-trifluoroethyl)-2-(3,3,3-trifluoro-2-methylpropyl)-1,9-dihydro-6H-purin-6-one;
1-(4-chlorophenyl)-9-(2,2,2-trifluoroethyl)-2-(4,4,4-trifluoro-3-methylbutyl)-1,9-dihydro-6H-purin-6-one;
1-(4-chlorophenyl)-9-(2,2,2-trifluoroethyl)-2-(5,5,5-trifluoropentyl)-1,9-dihydro-6H-purin-6-one;
9-(2,2-difluoroethyl)-1-(4-fluorophenyl)-2-(4,4,4-trifluoro-2-methylbutyl)-1,9-dihydro-6H-purin-6-one;
9-(2,2-difluoroethyl)-1-(4-fluorophenyl)-2-(4,4,4-trifluoro-3-methylbutyl) 1,9-dihydro-6H-purin-6-one;
9-(2,2-difluoroethyl)-1-(4-fluorophenyl)-2-(5,5,5-trifluoropentyl)-1,9-dihydro-6H-purin-6-one;
1-(4-chlorophenyl)-9-ethyl-2-(4-methylcyclohexyl)-1,9-dihydro-6H-purin-6-one;
1-(4-chlorophenyl)-9-ethyl-2-(4-isopropylcyclohexyl)-1,9-dihydro-6H-purin-6-one;
2-(4-tert-butylcyclohexyl)-1-(4-chlorophenyl)-9-ethyl-1,9-dihydro-6H-purin-6-one;
1-(4-chlorophenyl)-9-ethyl-2-[4-(trifluoromethyl)cyclohexyl]-1,9-dihydro-6H-purin-6-one;
1-(4-chlorophenyl)-2-(4,4-difluorocyclohexyl)-9-ethyl-1,9-dihydro-6H-purin-6-one;
9-ethyl-1-(4-fluorophenyl)-2-[4-(trifluoromethyl)cyclohexyl]-1,9-dihydro-6H-purin-6-one;
1-(4-chlorophenyl)-9-methyl-2-[4-trifluoromethyl)cyclohexyl]-1,9-dihydro-6H-purin-6-one;
1-(4-fluorophenyl)-9-methyl-2-[4-(trifluoromethyl)cyclohexyl]-1,9-dihydro-6H-purin-6-one;
4-[9-methyl-6-oxo-2-(4,4,4-trifluoro-3-methylbutyl)-6,9-dihydro-1H-purin-1-yl]benzonitrile;
4-[9-methyl-6-oxo-2-(4,4,4-trifluoro-2-methylbutyl)-6,9-dihydro-1H-purin-1-yl]benzonitrile;
2-(2-chloroethyl)-1-(4-chlorophenyl)-9-ethyl-1,9-dihydro-6H-purin-6-one;
1-(4-chlorophenyl)-2-[2-(2,3-dihydro-1-benzofuran-2-yl)ethyl]-9-ethyl-1,9-dihydro-6H-purin-6-one;
1-(4-chlorophenyl)-2-[2-(2,3-dihydro-1-benzofuran-2-yl)ethyl]-9-methyl-1,9-dihydro-6H-purin-6-one;
2-[(E)-2-(1-benzofuran-2-yl)vinyl]-1-(4-chlorophenyl)-9-methyl-1,9-dihydro-6H-purin-6-one;
2-(1-benzothien-3-ylmethyl)-1-(4-chlorophenyl)-9-ethyl-1,9-dihydro-6H-purin-6-one;
2-(1-benzothien-3-ylmethyl)-1-(4-chlorophenyl)-9-methyl-1,9-dihydro-6H-purin-6-one;
1-(4-chlorophenyl)-2-(3-chloropropyl)-9-ethyl-1,9-dihydro-6H-purin-6-one;
1-(4-chlorophenyl)-9-ethyl-2-[(3-methyl-1-benzothien-2-yl)methyl]-1,9-dihydro-6H-purin-6-one;
1-(3,4-difluorophenyl)-9-methyl-2-[(3-methyl-1-benzothien-2-yl)methyl]-1,9-dihydro-6H-purin-6-one;
9-ethyl-1-(4-fluorophenyl)-2-[5-(trifluoromethyl)-2,3-dihydro-1H-inden-2-yl]-1,9-dihydro-6H-purin-6-one;
1-(4-chlorophenyl)-2-(2,3-dihydro-1-benzofuran-3-ylmethyl)-9-ethyl-1,9-dihydro-6H-purin-6-one;
1-(4-chlorophenyl)-2-(2,3-dihydro-1-benzofuran-3-ylmethyl)-9-methyl-1,9-dihydro-6H-purin-6-one;
1-(3,4-difluorophenyl)-9-methyl-2-[5-(trifluoromethyl)-2,3-dihydro-1H-inden-2-yl]-1,9-dihydro-6H-purin-6-one;
   and pharmaceutically acceptable salts and tautomers thereof.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂F, CHF₂, CH₂CH₂F, CH₂CHF₂, CH₂CF₃, OCF₃, OCH₂CH₂F, OCH₂CHF₂ or OCH₂CF₃, and most especially CF₃ and OCF₃. A further preferred group is chloroC₁₋₆alkyl, for example CCl₃, CH₂Cl, CHCl₂, CH₂CH₂Cl, CH₂CHCl₂, CH₂CCl₃, especially CH₂Cl.

The cycloalkyl groups referred to herein may represent, for example, cyclopropyl, cyclobutyl, cyclopentyl or cyclohexyl. Such groups also include, for example, cyclopropylmethyl and cyclohexylmethyl.

As used herein, the terms “alkenyl” and “alkynyl” as a group or part of a group means that the group is straight or branched. Examples of suitable alkenyl groups include vinyl and alkyl. A suitable alkynyl group is acetylene or propargyl.

When used herein, the term “halogen” means fluorine, chlorine, bromine and iodine. The most preferred halogens are fluorine and chlorine, especially chlorine.

Examples of 6-membered saturated rings are morpholine, piperidine and piperazine. A further saturated ring is tetrahydrothiopyranyl.

Examples of 6-membered heteroaromatic rings are pyridine, pyrimidine, pyrazine, pyridazine and triazine.

Examples of 5-membered heteroaromatic rings are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

Examples of 9- or 10-membered fused bicyclic heteroaromatic rings include benzofuran, benzothiophene, benzimidazole, benzoxazole, benzothiazole, benzisothiazole, quinoline, isoquinoline and cinnoline.

Examples of 8 to 10 membered fused bicyclic partially saturated ring include tetrahydrobenzothiazolyl, tetrahydronaphthalenyl, dihydrobenzofuranyl, dihydroindenyl, and dihydrochromenyl.

An example of a 9-membered fused bicyclic partially saturated ring is tetrahydrobenzothiazolyl.

In a further aspect of the present invention, the compounds of formula I, IA, IAA, IIA, IB, IIB, or IC may be prepared in the form of a pharmaceutically acceptable salt, especially an acid addition salt.

For use in medicine, the salts of the compounds of formula I, IA, IAA, IIA, IB, IIB or IC will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. A further salt is the acid addition salt with benzenesulfonic acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula I, IA, IAA, IIA, IB, IIB or IC with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula I, IA, IAA, IIA, IB, IIB or IC above. In general, such N-oxides may be formed on any available nitrogen atom. The N-oxides may be formed by conventional means, such as reacting the compound of formula I, IA, IAA, IIA, IB, IIB or IC with Oxone® in the presence of wet alumina.

The present invention includes within its scope prodrugs of the compounds of formula I, IA, IAA, IIA, IB, IIB or IC above. In general, such prodrugs will be functional derivatives of the compounds of formula I, IA, IAA, IIA, IB, IIB or IC which are readily convertible in vivo into the required compound of formula I, IA, IAA, IIA, IB, IIB or IC. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I, IA, IAA, IIA, IB, IIB or IC and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula I, IA, IAA, IIA, IB, IIB or IC may also exist in tautomeric forms and the invention includes within its scope both mixtures and separate individual tautomers.

The compounds may exist in different isomeric forms, all of which are encompassed by the present invention.

The present invention further provides pharmaceutical compositions comprising one or more compounds of formula I, IA, IAA, IIA, IB, IIB or IC in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula I required for use in any treatment will vary not only with the particular compounds or composition selected but also with the route of administration, the nature of the condition being treated, and the age and condition of the patient, and will ultimately be at the discretion of the attendant physician.

The invention further provides a compound of formula I, IA, IAA, IIA, B, IIB or IC as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynecological pain, for example, dysmenorrhea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, for example stump pain and phantom limb pain, peripheral neuropathies, tic douloureux, atypical facial pain, nerve root damage, and arachnoiditis; itching conditions including pruritis, itch due to hemodialysis, contact dermatitis, vulvar vestibulitis, insect bites and skin allergies; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy, post-herpetic neuralgia, causalgia (reflex sympathetic dystrophy—RSD, secondary to injury of a peripheral nerve), neuritis (including sciatic neuritis, peripheral neuritis, polyneuritis, optic neuritis, postfebrile neuritis, migrating neuritis, segmental neuritis and Gombault's neuritis, neuronitis, neuralgias (including cervicobrachial neuralgia, cranial neuralgia, geniculate neuralgia, glossopharynigial neuralgia, migranous neuralgia, idiopathic neuralgia, intercostals neuralgia, mammary neuralgia, mandibular joint neuralgia, Morton's neuralgia, nasociliary neuralgia, occipital neuralgia, red neuralgia, Sluder's neuralgia, splenopalatine neuralgia, supraorbital neuralgia and vidian neuralgia), AIDS-related neuropathy and MS related neuropathy; “non-painful” neuropathies; oral neuropathic pain; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence (including overflow incontinence, urge incontinence and stress incontinence) including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis and cough; autoimmune diseases; immunodeficiency disorders; hot flushes; postmastectomy pain syndrome; dental pain, such as toothache and denture pain; reflex sympathetic dystrophy; trigeminal neuralgia; fibromyalgia; Guillain-Barre syndrome; meralgia paresthetica; burning-mouth syndrome; Charcot's pains; intestinal gas pains; menstrual pain; hemorrhoidal pain; dyspeptic pains; angina; homotopic and heterotopic pain; pain associated with venom exposure; other trauma associated pain such as pain from cuts, bruises, broken bones and burns); and carpel tunnel syndrome. The compounds of the present invention may also be used to treat depression, hiccups and obesity. They may also be used to treat gastro-oesophageal reflux disease (GERD), particularly the pain associated with GERD. The compounds may also be used for the treatment or prevention of treatment and prevention of diabetes mellitus, including Type I diabetes (Insulin Dependent Diabetes Mellitus), Type II diabetes (Non-Insulin Dependent Diabetes Mellitus), gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), Type A insulin resistance syndrome, Type B insulin resistance syndrome, lipatrophic diabetes, and diabetes induced by (3-cell toxins).

Thus, according to a further aspect, the present invention provides a compound of formula I, IA, IAA, IIA, IB, IIB or IC for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula I, IA, IAA, IIA, IB, IIB or IC or a composition comprising a compound of formula I, IA, IAA, IIA, B, IIB or IC.

According to a further or alternative aspect, the present invention provides a compound of formula I, IA, IAA, IIA, IB, IIB or IC for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula I, IA, IAA, IIA, IB, IIB or IC or a composition comprising a compound of formula I, IA, IAA, IIA, IB, IIB or IC.

According to a further aspect of the present invention, it may be desirable to treat any of the aforementioned conditions with a combination of a compound according to the present invention and one or more other pharmacologically active agents suitable for the treatment of the specific condition. The compound of formula I, IA, IAA, IIA, IB, IIB or IC and the other pharmacologically active agent(s) may be administered to a patient simultaneously, sequentially or in combination.

Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin, asthma treatments (such as θ₂-adrenergic receptor agonists or leukotriene D₄ antagonists (e.g. montelukast), gold compounds, corticosteroids, methotrexate, tumor necrosis (TNF) receptor antagonists, anti-TNF alpha antibodies, anti-C5 antibodies and interluekin-1 (IL-1) receptor antagonists.

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib, tilicoxib, flurbiprofen, naproxen sodium, combinations of diclofenac sodium and misoprostol, oxaprozin, diflunisal, fenoprofen, calcium, sodium nabumetone, sulfasalazine, tolmetin sodium, hydroxychloroquine, acetylsalicylic acid, sodium salicylate, choline and magnesium salicylates, salsalate, cortisone, dexamethasone, methylprednisolone, prednisolone, prednisolone sodium phosphate, prednisone and lumiracoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene, pentazocine, alphaprodine, anileridine, bezitramide, diacetyldihydromorphine, diphenoxylate, ethylmorphine, heroin, isomethadone, levomethorphan, levorphanol, metazocine, methorphan, metopon, opium extracts, opium fluid extracts, powdered opium, granulated opium, raw opium, tincture of opium, oxycodone, paregoric, pethidine, phenazocine, piminodine, racemethorphan, racemorphan, thebaine, acetorphine, acetyldihydrocodeine, acetylmethadol, allylprodine, alphracetylmethadol, alphameprodine, alphamethadol, benzethidine, benzylmorphine, betacetylmethadol, betameprodine, betamethadol, betaprodine, clonitazene, codeine methylbromide, codeine-N-oxide, cyprenorphine, desomorphine, dextromoramide, diampromide, diethylthiambutene, dihydromorphine, dimenoxadol, dimepheptanol, dimethylthiamubutene, dioxaphetyl, butyrate, dipipanone, drotebanol, ethanol, ethylmethylthiambutene, etonitazene, etorphine, etoxeridine, furethidine, hydromorphinol, hydroxypethidine, ketobemidone, levomoramide, levophenacylmorphan, methyldesorphine, methyldihydromorphine, morpheridine, morphine methylpromide, morphine methylsulfonate, morphine-N-oxide, myrophin, naloxone, nalbuyphine, naltyhexone, nicocodeine, nicomorphine, noracymethadol, norlevorphanol, normethadone, normorphine, norpipanone, pentazocaine, phenadoxone, phenampromide, phenomorphan, phenoperidine, piritramide, pholcodine, proheptazoine, properidine, propiran, racemoramide, thebacon and trimeperidine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Thus, for example, for the treatment or prevention of cough, a compound of the present invention may be used in conjunction with other medication designed to treat this condition, such as antibiotics, anti-inflammatory agents, cystinyl leukotrienes, histamine antagonists, corticosteroids, opioids, NMDA antagonists, proton pump inhibitors, nociceptin, neurokinin (NK1, NK2 and NK3) and bradykinin (BK1 and BK2) receptor antagonists, cannabinoids, blockers of Na+-dependent channels and large conductance Ca(2+)-dependent K+-channel activators. Specific agents include dexbrompheniramine plus pseudoephedrine, loratadine, oxymetazoline, ipratropium, albuterol, beclomethasone, morphine, codeine, pholcodeine and dextromethorphan.

Thus, for example, for the treatment or prevention of urinary incontinence, a compound of the present invention may be used in conjunction with other medication designed to treat this condition, such as estrogen replacement therapy, progesterone congeners, electrical stimulation, calcium channel blockers, antispasmodic agents, cholinergic antagonists, antimuscarinic drugs, tricyclic antidepressants, SNRIs, beta adrenoceptor agonists, phosphodiesterase inhibitors, potassium channel openers, nociceptin/orphanin FQ (OP4) agonists, neurokinin (NK1 and NK2) antagonists, P2X3 antagonists, musculotrophic drugs and sacral neuromodulation. Specific agents include oxybutinin, emepronium, tolterodine, flavoxate, flurbiprofen, tolterodine, dicyclomine, propiverine, propantheline, dicyclomine, imipramine, doxepin, duloxetine and 1-deamino-8-D-arginine vasopressin.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

Compounds of formula I can be prepared by reacting a compound of formula II:

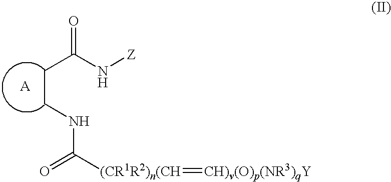

wherein n, p, q, v, A, R¹, R², R³, Y and Z are as defined above, with a cyclising agent such as phosphorus oxychloride, generally at a temperature of 100° to 120° C. The reaction may also be carried out in a solvent or a mixture of solvents such as toluene at about 110° C. or tetrahydrofuran at 65° C., or with a base such as sodium ethoxide in a solvent such as ethanol at room temperature to 50° C. Alternatively, a cyclising agent such as polyphosphoric acid (PPA) may be used, generally at about 150° C.

Compounds of formula II can be prepared by reacting a compound of formula III with a compound of formula IV:

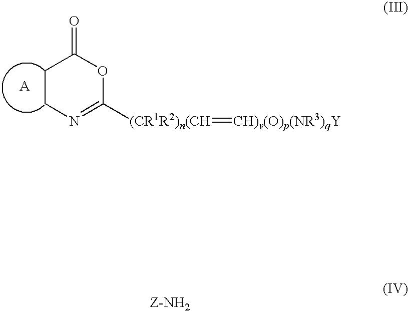

wherein n, p, q, v, A, R¹, R², R³, Y and Z are as defined above. The reaction is generally carried out in the presence of a weak acid such as pivalic or acetic acid in a solvent such as toluene at a temperature of about 110° C.

Compounds of formula III can be prepared by reacting a compound of formula V:

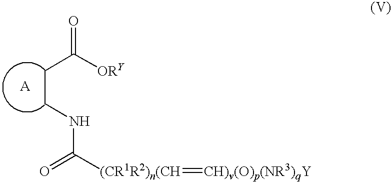

wherein n, p, q, v, A, R¹, R², R³ and Y are as defined above and R^{Y} is a C₁₋₆alkyl group such as ethyl, with a cyclising agent such as phosphorous oxychloride, generally at about 100 to 120° C. The reaction may also be carried out in a solvent or mixture of solvents such as toluene at about 110° C. or tetrahydrofuran at about 65° C., or with a base such as sodium ethoxide in a solvent such as ethanol at room temperature to 50° C.

Compounds of formula V can be prepared by reacting a compound of formula VI with a compound of formula VII:

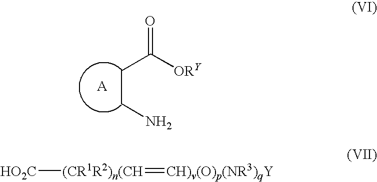

wherein n, p, q, v, A, R¹, R², R³, Y and R^{Y} are as defined above. The reaction is generally carried out in a solvent such as trimethylacetic anhydride at about 85° C. An amide coupling agent such as EDC or HATU may be used.

In an alternative process, compounds of formula I wherein n, p, q and v are zero and Y is an aromatic ring (Ar) can be prepared by reacting a compound of formula VIII with a compound of formula IX:

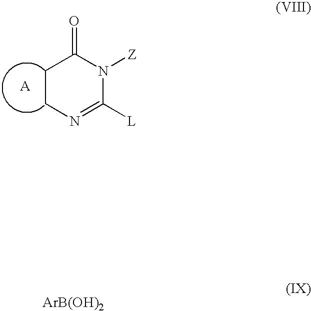

wherein A and Z are as defined above, Ar is an aromatic ring as defined for Y above and L is a leaving group such as chlorine. The reaction is generally carried out in a solvent such as tetrahydrofuran (THF) at about 150° C. under pressure in a microwave reactor or at reflux.

Compounds of formula VIII wherein L is chlorine can be prepared by reacting a compound of formula X:

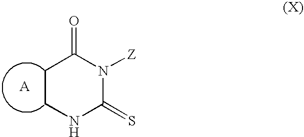

wherein A and Z are as defined above with a chlorinating agent such as POCl₃, generally at reflux.

Compounds of formula X can be prepared by reacting a compound of formula XI:

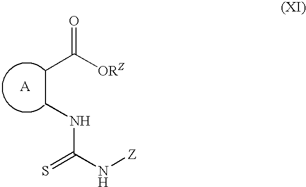

wherein A and Z are as defined above and R^{Z} is a C₁₋₆alkyl group such as ethyl, with a base such as potassium hydroxide or sodium hydroxide, generally in a solvent such as water at a temperature from about 50 to 80° C.

Compounds of formula XI can be prepared by reacting a compound of formula XII with a compound of formula XIII:

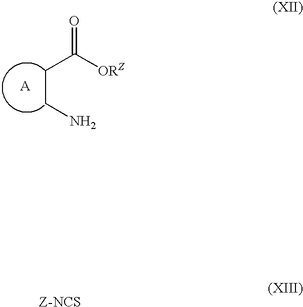

wherein A, Z and R^{Z} are as defined above. The reaction is generally carried out in a solvent such as pyridine at about 45° C.

Alternatively, compounds of formula I wherein n is two and v is zero or n is zero and v is one; and p and q are both zero and R¹ and R² are both hydrogen can be prepared by hydrogenation of a compound of formula XIV:

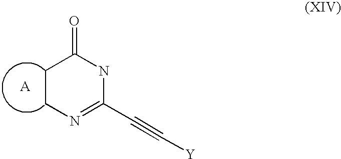

wherein A and Y are as defined above, generally in the presence of catalysts such as palladium on carbon and in solvents such as methanol and ethyl acetate.

Compounds of formula XIV can be prepared by reacting a compound of formula VIII with a compound of formula XV:

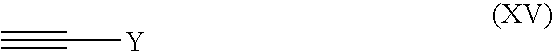

wherein Y is as defined above, generally in the presence of a catalyst such as copper(I)iodide and bis(triphenylphosphino)palladium(II)dichloride, in solvents such as triethylamine and N,N-dimethylacetamide at a temperature of about 110° C.

Compounds of formula I can alternatively be prepared by reacting a compound of formula XVI with a compound of formula VII:

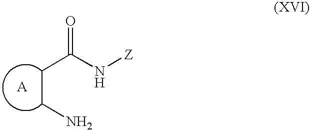

wherein A and Z are as defined above, generally in the presence of a cyclising agent such as polyphosphoric acid at a temperature of about 150° C.

Compounds of formula XVI can be prepared by reacting a compound of formula IV with a compound of formula VI, generally in the presence of trimethylaluminium in a solvent such as 1,2-dichloroethane at about 90° C. to reflux.

Compounds of formula II can alternatively be prepared by reacting a compound of formula XVI with a compound of formula VII. The reaction can be carried out in the presence of bis(2-oxo-3-oxazolidinyl)phosphinic chloride (BOP-Cl), a base such as N,N-di-isopropylethylamine(iPrNEt₂), in a solvent such as dichloroethane (DCE) at a temperature of about 160° C.

Compounds of formula II can alternatively be prepared by reacting a compound of formula XVI with a compound of formula XVII:

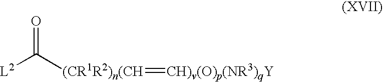

wherein n, p, q, v, R¹, R², R³ and Y are as defined above and L² is a halogen such as chlorine. The reaction can generally be carried out in the presence of a base such as i-PrNEt₂, in a solvent such as THF at about 100° C.

Compounds of formulae IA, IAA, IIA, IB, IIB and IC can be prepared by analogous methods as described above for formula I mutatis mutandis.

Where the synthesis of intermediates and starting materials is not described these compounds are commercially available or can be made from commercially available compounds by standard methods, or by extension from the Descriptions and Examples herein.

Compounds of formula I may be converted to other compounds of formula I by known methods or by methods described in the Descriptions and Examples.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### Description 1

### Ethyl 5-amino-1-ethyl-1H-imidazole-4-carboxylate

To a solution of ethyl 3-nitriloalaninate (Synthesis, 1996, 11, 1325; 25 g, 0.195 mol) in MeCN (400 ml) was added triethylorthoformate (32.5 ml, 28.9 g, 0.195 mol) and the resulting solution heated to 90° C. After 70 min the solution was cooled to room temperature and a solution of ethylamine (2 M in tetrahydrofuran, 98 ml, 0.195 mol) added and the reaction stirred at RT for 18 h. The reaction was condensed in vacuo to a viscous oil then taken up in hydrochloric acid (1 N, 200 ml). The aqueous layer was washed with dichloromethane (2×200 ml, 1×100 ml). The aqueous layer was neutralised by the addition of solid sodium bicarbonate (˜25 g) and then extracted with dichloromethane (5×200 ml). The organic layers were combined, dried over MgSO₄ and condensed in vacuo to give a brown/red solid residue. The residue was slurried in ethyl acetate (50 ml), filtered, and the solid rinsed with diethyl ether and dried to give ethyl 5-amino-1-ethyl-1H-imidazole-4-carboxylate (13.0 g, 36%). ¹H NMR (360 MHz, CDCl₃) δ 7.05 (1H, s), 4.85 (2H, br. s), 4.34 (2H, q, J7), 3.79 (2H, q, J7), 1.43 (3H, t, J7), 1.39 (3H, t, J 7).

### Description 2

### 1-(4-Chlorophenyl)-9-ethyl-2-thioxo-1,2,3,9-tetrahydro-6H-purin-6-one hydrochloride

Description 1 (0.62 g, 3.4 mmol) and 4-chlorophenyl isothiocyanate (0.58 g, 3.4 mmol) were stirred in pyridine (2 ml) at 45° C. for 18 h. The suspension was cooled and diluted by the addition of ice. When the ice had melted the reaction was extracted into ethyl acetate. The organic layer was dried over MgSO₄ and evaporated in vacuo to give a mixture of the symmetrical thiourea N,N′-bis(4-chlorophenyl)thiourea and ethyl 5-({[(4-chlorophenyl)amino]carbonothioyl}amino)-1-ethyl-1H-imidazole-4-carboxylate (1.12 g). The solid was slurried in 1% aqueous sodium hydroxide solution (20 ml) and heated at 90° C. for 16 h. The reaction was filtered, the filtrate evaporated in vacuo, and the residue was diluted with methanol and loaded onto a strong cation exchange (SCX) cartridge. The cartridge was washed with methanol and dichloromethane and then the product eluted with 2 M methanolic ammonia. After drying, this gave the title compound (756 mg, 72%). ¹H NMR (400 MHz, CD₃OD) δ 7.76 (1H, s), 7.43 (2H, d, J8.5), 7.13 (2H, d, J8.7), 4.16 (2H, q, J7.3), 1.45 (3H, t, J7.2); m/z (ES⁺) 307, 309 (M+H⁺).

### Description 3

### 2-Chloro-1-(4-chlorophenyl)-9-ethyl-1,9-dihydro-6H-purin-6-one

Description 2 (860 mg, 2.5 mmol) was suspended in a large excess of phosphorous oxychloride (>20 eq) and heated to 135° C. for 36 h. The reaction mixture was cooled, evaporated in vacuo, and azeotroped twice with toluene. The resulting sticky brown oil was dissolved in dichloromethane then neutralised with sat. NaHCO₃ (aq). The dichloromethane layer was dry loaded onto a silica flash column, eluting product with ethyl acetate/dichloromethane (1:1) to give the title compound as a pale yellow solid (426 mg, 55%). ¹H NMR (500 MHz, CDCl₃) δ 7.79 (1H, s), 7.52 (2H, d, J8.6), 7.21 (2H, d, J8.6), 4.23 (2H, q, J7.3), 1.56 (3H, t, J7.3); m/z (ES⁺) 309, 311 (M+H⁺).

### Description 4

### Ethyl 5-amino-1-methyl-1H-imidazole-4-carboxylate

To a solution of ethyl 3-nitriloalaninate (Synthesis, 1996, 11, 1325; 20 g, 0.156 mol) in MeCN (400 ml) was added triethylorthoformate (26 ml, 23.2 g, 0.156 mol) and the resulting solution heated to 90° C. After 1 h the solution was cooled to room temperature and a solution of methylamine (8 M in ethanol, 20 ml, 0.156 mol) added and the reaction stirred at RT for 18 h. The reaction was condensed in vacuo to a viscous oil then taken up in hydrochloric acid (1 N, 180 ml). The aqueous layer was washed with dichloromethane (2×200 ml, 1×100 ml). The aqueous layer was neutralised by the addition of solid sodium bicarbonate (˜20 g) and then extracted with dichloromethane (5×200 ml). The organic layers were combined, dried over MgSO₄ and condensed in vacuo to give a brown/red solid residue. The residue was slurried in ethyl acetate (40 ml) with sonication, filtered, then the solid rinsed with ether and dried to give ethyl 5-amino-1-methyl-1H-imidazole-4-carboxylate (9.88 g, 37%). ¹H NMR (400 MHz, DMSO) δ 7.08 (1H, s), 5.94 (2H, s), 4.15 (2H, q, J7.1), 3.39 (3H, s), 1.24 (3H, t, J7.1).

### Description 5

### 1-(4-Chlorophenyl)-9-methyl-2-thioxo-1,2,3,9-tetrahydro-6H-purin-6-one hydrochloride

Description 4 (4.5 g, 26.6 mmol) and 4-chlorophenyl isothiocyanate (4.5 g, 26.6 mmol) were stirred in pyridine (22 ml) at 45° C. for 18 h. The suspension was cooled and diluted by the addition of ice. When the ice had melted the reaction was filtered, the product rinsed with water and diethyl ether to give ethyl 5-({[(4-chlorophenyl)amino]carbonothioyl}amino)-1-methyl-1H-imidazole-4-carboxylate. The solid was slurried in 1% aqueous sodium hydroxide solution (150 ml) and heated at 80° C. for 30 mins. The reaction was filtered to remove insoluble impurities and then acidified to pH˜5 using hydrochloric acid (5 N), causing a thick white suspension to form The mixture was aged for 30 minutes and filtered. The solid was rinsed with water then diethyl ether and dried to give the title compound as a white solid (5.28 g, 68%). ¹H NMR (360 MHz, DMSO) δ 7.58 (1H, s), 7.37 (2H, m), 7.06 (1H, br. s), 6.96 (2H, m), 3.54 (3H, s); m/z (ES⁺) 293, 295 (M+H⁺).

### Description 6

### 2-Chloro-1-(4-chlorophenyl)-9-methyl-1,9-dihydro-6H-purin-6-one

Prepared from Description 5 according to the procedure outlined in Description 3.

¹H NMR (360 MHz, DMSO) δ 8.14 (1H, s), 7.64 (2H, d, J8.6), 7.52 (2H, d, J8.6), 3.76 (3H, s); m/z (ES⁺) 295, 297 (M+H⁺).

### Description 7

### Ethyl 1-methyl-5-[(4,4,4-trifluorobutanoyl)amino]-1H-imidazole-4-carboxylate

A mixture of Description 4 (510 mg, 3.02 mmol), 4,4,4-trifluorobutanoic acid (1.73 g, 12.2 mmol) and trimethylacetic anhydride (4 ml) was heated at 85° C. for 24 h, after which time tlc indicated the reaction was complete. The mixture was cooled to room temperature, poured into ether (50 ml) and crystallisation was induced by scratching. The product was collected by filtration, washed with a small quantity of ether and dried to give the title compound as a pale brown solid (588 mg, 67%). ¹H NMR (360 MHz, CDCl₃) δ 8.27 (1H, s), 7.34 (1H, s), 4.36 (2H, q, J7.1), 3.60 (3H, s), 2.80-2.70 (2H, app. t, J7.5), 2.62-2.52 (2H, m), 1.39 (3H, t, J7.1); m/z (ES⁺) 294 (M+H⁺).

### Description 8

### 3-Methyl-5-(3,3,3-trifluoropropyl)imidazo[4,5-d][1,3]oxazin-7(3H)-one

Description 7 (585 mg, 2.00 mmol) was suspended in toluene (6 ml) and phosphorus oxychloride (373 μL, mmol) was added. The mixture was then heated at reflux for 6 h, after which time tlc indicated the reaction was complete. The mixture was cooled to room temperature, the toluene evaporated and the residue purified by flash column chromatography (eluant: 5% MeOH in CH₂Cl₂) to give the title compound (325 mg, 66%). ¹H NMR (400 MHz, CDCl₃) δ 7.72 (1H, s), 3.78 (3H, s), 3.03 (2H, app. t, J 7.9), 2.74-2.62 (2H, m); m/z (ES⁺) 248 (M+H⁺).

### Description 9

### Ethyl 1-methyl-5-[(5,5,5-trifluoropentanoyl)amino]-1H-imidazole-4-carboxylate

A mixture of Description 4 (670 mg, 3.96 mmol), 5,5,5-trifluoropentanoic acid (prepared according to EP96995; 625 mg, 4.00 mmol) and trimethylacetic anhydride (6 ml) was heated at 85° C. for 24 h, after which time tlc indicated the reaction was complete. The mixture was cooled to room temperature, poured into ether (70 ml) and crystallisation was induced by scratching. The product was collected by filtration, washed with a small quantity of ether and dried to give the title compound as a pale brown solid (680 mg, 56%). ¹H NMR (500 MHz, CDCl₃) δ 8.23 (1H, s), 7.33 (1H, s), 4.36 (2H, q, J7.1), 3.62 (3H, s), 2.58 (2H, br. t, J7.0), 2.27-2.17 (2H, m), 2.02 (2H, quin, J7), 1.39 (3H, t, J7.1); m/z (ES⁺) 308 (M+H⁺).

### Description 10

### 3-Methyl-5-(4,4,4-trifluorobutyl)imidazo[4,5-d][1,3]oxazin-7(3H)-one

Prepared from Description 9 according to the procedure of Description 8. ¹H NMR (400 MHz, CDCl₃) δ 7.69 (1H, s), 3.78 (3H, s), 2.84 (2H, t, J7.4), 2.31-2.07 (4H, m).

### Description 11

### 5-(2-Cyclohexylethyl)-3-methylimidazo[4,5-d][1,3]oxazin-7(3H)-one

Prepared from Description 4 and 3-cyclohexylpropionic acid according to the procedures of Descriptions 7 and 8 respectively. ¹H NMR (360 MHz, CDCl₃) δ 7.67 (1H, s), 3.77 (3H, s), 2.73 (2H, app. t, J8.0), 1.80-1.60 (7H, m), 1.40-1.10 (4H, m), 1.05-0.85 (2H, m).

### Description 12

### Ethyl 1-methyl-5-[(4-phenylbutanoyl)amino]-1H-imidazole-4-carboxylate

Prepared from Description 4 and 4-phenylbutyric acid according to the procedure of Description 7, with crystallisation from ether-hexane rather than ether. ¹H NMR (360 MHz, DMSO) δ 9.82 (1H, s), 7.66 (1H, s), 7.31-7.16 (5H, m), 4.15 (2H, q, J7.1), 3.42 (3H, s), 2.64 (2H, t, J7.5), 2.37 (2H, t, J7.4), 1.94-1.86 (2H, m), 1.22 (3H, t, J7.1).

### Description 13

### 3-Methyl-5-(3-phenylpropyl)imidazo[4,5-d][1,3]oxazin-7(3H)-one

Description 12 (709 mg, 2.3 mmol) was suspended in toluene (10 ml) and phosphorus oxychloride (419 μL, 4.6 mmol) was added. The mixture was then heated at reflux for 3 h, after which time tlc indicated the reaction was complete. The reaction was cooled to room temperature, and the mixture was partitioned between ethyl acetate (15 ml) and sat aqueous potassium carbonate solution (15 ml). The layers were separated, the aqueous phase extracted with more ethyl acetate (20 ml) and the combined organic phases were dried (MgSO₄) and evaporated. The residue was purified by flash column chromatography (eluant: 5% MeOH in CH₂Cl₂) to give the title compound (641 mg, 100%). ¹H NMR (360 MHz, DMSO) δ 8.08 (1H, s), 7.30-7.16 (5H, m), 3.70 (3H, s), 2.75-2.67 (4H, m), 2.03 (2H, t, J7.5); m/z (ES⁺) 270 (M+H⁺).

### Description 14

### N-(4-Chlorophenyl)-1-methyl-5-[(5,5,5-trifluoropentanoyl)amino]-1H-imidazole-4-carboxamide

A mixture of Description 10 (235 mg, 0.90 mmol), 4-chloroaniline (229 mg, 1.80 mmol), pivalic acid (470 mg) and toluene (5 ml) was heated at reflux for 8 h, after which time the reaction was complete. The cooled reaction mixture was evaporated. Trituration of the residue with ether-hexane gave some product which was collected by filtration; the filtrate was then evaporated and purified by flash column chromatography (eluant 5% MeOH in CH₂Cl₂) and the product-containing fractions evaporated and triturated with ether-hexane. The solids obtained by trituration were combined to give the title compound (193 mg, 55%). ¹H NMR (400 MHz, CDCl₃) 8.74 (1H, s), 8.56 (1H, s), 7.58 (2H, d, J8), 7.31 (2H, d, J8), 3.65 (3H, s), 2.60 (2H, t, J7.3), 2.29-2.17 (2H, m), 2.05-1.99 (2H, m).

### Description 15

### N-(4-Chlorophenyl)-1-methyl-5-[(4-phenylbutanoyl)amino]-1H-imidazole-4-carboxamide

Prepared from Description 13 and 4-chloroaniline according to the procedure of Description 14.

### Description 16

### 5-Amino-N-(4-chlorophenyl)-1-methyl-1H-imidazole-4-carboxamide

Trimethylaluminium (2 M solution in hexane; 8.9 ml, 17.7 mmol) was added dropwise to a solution of 4-Chloroaniline (1.13 g, 8.87 mmol) in 1,2-dichloroethane (18 ml) over 5 mins at RT under an atmosphere of N₂. The resulting suspension was stirred for 30 mins before Description 4 (1.00 g, 5.91 mmol) was added and the slurry heated to reflux for 6 h. On cooling, the solution was diluted with CH₂Cl₂ (60 ml) and saturated aqueous sodium potassium tartrate (60 ml) was added, followed by saturated aqueous ammonium chloride solution (20 ml) and MeOH (10 ml). The mixture was stirred vigorously for 1 h and then allowed to settle for 1 h before separation of the phases. The aqueous phase was extracted with 8% MeOH/DCM (50 ml) and the combined organic extracts washed with 1M sodium potassium tartrate (150 ml dried over MgSO₄, filtered and concentrated in vacuo to give an orange solid. This was slurried in diethyl ether and the mixture filtered. The residue was washed with ether and dried by a stream of air for 1 h to give the title compound as a golden solid (1.38 g, 93%). ¹H NMR (500 MHz, DMSO): δ 9.44 (1H, s), 7.84 (2H, d, J8.8), 7.30 (2H, d, J8.8), 7.19 (1H, s), 5.98 (2H, s), 3.43 (3H, s).

### Description 17

### 5-amino-1-methyl-N-phenyl-1H-imidazole-4-carboxamide

Prepared from Description 4 and aniline according to the procedure of Description 16. ¹H NMR (500 MHz, DMSO): δ 9.21 (1H, s), 7.77 (2H, d, J7.9), 7.26 (2H, t, J7.8), 7.18 (1H, s), 6.97 (1H, t, J7.3), 5.95 (2H, s), 3.43 (3H, s).

### Description 18

### 5-amino-N-(4-chlorophenyl)-1,3-thiazole-4-carboxamide

Prepared from ethyl 5-amino-1,3-thiazole-4-carboxylate (Tetrahedron 1985, 41, 5989) and 4-chloroaniline according to the procedure of Description 16, except that the reaction time was reduced to 3 h at 90° C. and the product extracted with CH₂Cl₂ (instead of 8% MeOH/CH₂Cl₂) and washed with 10% Et₂O/hexane (instead of Et₂O). ¹H NMR (500 MHz, DMSO): δ 9.83 (1H, s), 8.08 (1H, s), 7.85 (2H, d, J 8.9), 7.35 (4H, m).

### Description 19

### Ethyl 5-amino-1-propyl-1H-imidazole-4-carboxylate

Prepared according to the method of Description 1, using n-propylamine instead of ethylamine. ¹H NMR (360 MHz, DMSO) δ 7.10 (1H, s), 5.89 (2H, s), 4.14 (2H, q, J7.1), 3.75 (2H, t, J7.0), 1.63 (2H, q, J 7), 1.23 (3H, t, J7.1), 0.83 (3H, t, J7.4); m/z (ES⁺) 198 (M+H⁺).

### Description 20

### Ethyl 5-amino-1-cyclopropyl-1H-imidazole-4-carboxylate

Prepared according to the method of Description 1, using cyclopropylamine instead of ethylamine. ¹H NMR (360 MHz; CDCl₃) 5.09 (2H, s), 4.33 (2H, q, J7), 3.00-2.94 (1H, m), 1.38 (3H, t, J7), 1.2-1.0 (2H, m), 1.0-0.8 (2H, m). m/z (ES⁺) 196 (M+H⁺).

### Description 21

### Ethyl 5-amino-1-(2,2,2-trifluoroethyl)-1H-imidazole-4-carboxylate

Prepared according to the method of Description 1, using 2,2,2-trifluoroethylamine instead of ethylamine. ¹H NMR (360 MHz, d₆-DMSO) 1.24 (3H, t, J7.1); 4.17 (2H, q, J7.1); 4.92 (2H, q, J9.3); 6.32 (2H, br s); 7.18 (1H, s).

### Description 22

### 6,6,6-Trifluorohexanoic acid

Dimethyl malonate (3.45 g, 26.2 mmol) was added over 10 minutes to a suspension of sodium hydride (60% dispersion in paraffin; 1.15 g, 28.8 mmol) in THF (120 ml) at RT. 4-Bromo-1,1,1-trifluorobutane (5 g, 26.2 mmol) was then added at RT and the reaction stirred for 44 h. The mixture was then poured into water (200 ml) and extracted with ethyl acetate (2×200 ml). The organic phases were dried (Na₂SO₄) and evaporated. The residue was dissolved in ethanol (20 ml) and 4M aqueous NaOH (20 ml) added. The mixture was heated at 100° C. for 5 h, then the ethanol evaporated and the mixture acidified with 5N aqueous HCl. Extracted with ethyl acetate (2×100 ml) and the organic phases were dried (Na₂SO₄) and evaporated to give an oil which crystallised on standing. Trituration with hexane and collection by filtration gave the title compound as a white solid (2.84 g). ¹H NMR (360 MHz, DMSO): δ 12.7 (1H, s), 3.35-3.20 (2H, m), 2.35-2.15 (2H, m), 1.85-1.70 (2H, m), 1.55-1.40 (2H, m).

### Description 23

### Ethyl 1-methyl-5-{[4,5,5,5-tetrafluoro-4-(trifluoromethyl)pentanoyl]amino}-1H-imidazole-4-carboxylate

Prepared from Description 4 and 4,5,5,5-tetrafluoro-4-trifluoromethylpentanoic acid according to the procedure outlined in Description 7. ¹H NMR (400 MHz, DMSO) δ 10.08 (1H, s), 7.67 (1H, s), 4.16 (2H, q, J7.1), 3.42 (3H, s), 2.75-2.68 (2H, m), 2.65-2.55 (2H, m), 1.23 (3H, t, J7.1).

### Description 24

### 3-Methyl-5-[3,4,4,4-tetrafluoro-3-(trifluoromethyl)butyl]imidazo[4,5-d][1,3]oxazin-7(3H)-one

Description 23 (1.7 g, 0.65 mmol) was suspended in phosphorus oxychloride (36 ml, 387 mmol) and the reaction heated at 120° C. for 16 h, after which time tlc indicated complete reaction. The reaction was cooled to room temperature, the phosphorus oxychloride evaporated and the mixture was partitioned between ethyl acetate and sat. aqueous potassium carbonate solution. The layers were separated, the aqueous phase extracted with more ethyl acetate and the combined organic phases were dried (MgSO₄) and evaporated. The residue was purified by flash column chromatography on silica (eluent: 2.5% methanol in dichloromethane with 0.1% NH₃) to give the title compound (750 mg, 50%). ¹H NMR (400 MHz, DMSO) δ 8.12 (1H, s), 3.73 (3H, s), 3.09-3.01 (2H, m), 2.85-2.74 (2H, m).

### Description 25

### N-(4-chlorophenyl)-1-methyl-5-{[4,5,5,5-tetrafluoro-4-(trifluoromethyl)pentanoyl]amino}-1H-imidazole-4-carboxamide

Description 24 (330 mg, 0.95 mmol), 4-chloroaniline (242 mg, 1.90 mmol), acetic acid (400 □l) and toluene (5 ml) were heated at reflux for 16 h, after which time the reaction was complete by tlc. The cooled reaction mixture was evaporated and extracted into ethyl acetate, washed with water and brine and the organic phase dried (MgSO₄) and evaporated. Trituration of the residue with ether-hexane gave some product which was collected by filtration; the filtrate was then evaporated and purified by flash column chromatography on silica (eluant: 2.5% MeOH in dichloromethane with 0.1% NH₃) and the product containing fractions evaporated and triturated with ether-hexane. The solids obtained by trituration were combined to give the title compound (308 mg, 68%). ¹H NMR (400 MHz, DMSO) δ 10.20 (1H, s), 9.95 (1H, s), 7.86 (2H, d, J8.9), 7.76 (1H, s), 7.34 (2H, d, J8.5), 3.46 (3H, s), 2.75-2.69 (2H, m), 2.65-2.55 (2H, m).

### Description 26

### Ethyl 2-(trifluoromethyl)-1,3-thiazole-4-carboxylate

A solution of 2,2,2-trifluoroacetamide (7.12 g, 63 mmol) and Lawesson's Reagent (15.3 g, 37.8 mmol) in THF (anhydrous, 60 ml) was stirred at reflux for 18 hrs. The reaction mixture was cooled then ethyl bromopyruvate (8 ml, 63 mmol) added, and refluxed for 18 hrs. The reaction was cooled, evaporated in vacuo, and the resulting crude material extracted into ethyl acetate and washed with water. The organic fraction was dried (MgSO₄), and condensed to give a yellow/orange oil. The residue was purified by flash column chromatography on silica (eluent: 15% ethyl acetate in hexane) to provide the title compound as a clear oil (3 g, 21%). ¹H NMR 8 (400 MHz, CDCl₃) δ 8.39 (1H, s), 4.47 (2H, q, J7.1), 1.42 (3H, t, J7.2).

### Description 27

### 2-(Trifluoromethyl)-1,3-thiazole-4-carboxylic acid

A mixture of Description 26 (2.82 g, 12.5 mmol) and potassium hydroxide (1.7 g, 30 mmol), in methanol (2 ml) and water (7 ml) was heated at 80° C. for 16 h. The reaction was cooled, poured onto ice and acidified to pH 2 using conc. hydrochloric acid before extracting into ethyl acetate. The organic layer was dried (MgSO₄) and evaporated to give the title compound as a white solid (1.9 g, 77%). ¹H NMR (500 MHz, DMSO) δ 8.84 (1H, s).

### Description 28

### Ethel (2E)-3-[2-hydroxy-5-(trifluoromethyl)pyridin-3-yl]acrylate

To a suspension of Sodium hydride (2.09 g of a 60% dispersion in oil; 52.32 mmol) in anhydrous THF (100 ml), cooled in an ice bath was added dropwise a solution of triethyl phosphonoacetate (10.38 ml; 52.32 mmol) in anhydrous THF (50 ml). After complete addition the mixture was stirred until no further hydrogen evolution was observed (approximately 30 minutes). To this mixture was added a solution of 2-hydroxy-5-(trifluoromethyl)nicotinaldehyde (WO 2005/070133 A2; 5 g; 26.16 mmol) in anhydrous THF (100 ml) and the resulting mixture stirred at room temperature for 1 hour. The mixture was quenched by the careful addition of 2N HCl (250 ml) and EtOAc (500 ml) added (mostly a solid was observed suspended in the organic layer). The layers were separated, the EtOAc evaporated and the residue triturated with EtOAc/hexanes, filtered and dried to give a pale yellow solid (5.7 g, 83%). ¹H NMR (500 MHz d₆-DMSO) δ 1.25 (3H, t, J7.1), 4.17 (2H, q, J7.1), 7.17 (1H, d, J15.9), 7.58 (1H, d, J15.9), 8.05 (1H, s), 8.20 (1H, d, J2.1), 12.65 (1H, br s).

### Description 29

### Ethyl (2E)-3-[2-chloro-5-(trifluoromethyl)pyridin-3-yl]acrylate

A mixture of Description 28 (3.00 g; 11.49 mmol) and phosphorus oxychloride (30 ml) was warmed at 80° C. for 3 hours. The excess phosphorus oxychloride was removed by evaporation, and the residue dissolved in dichloromethane (70 ml). This mixture was stirred vigorously and treated portionwise with sat. aqueous NaHCO₃ (50 ml) and then stirred for 30 mins. The organic layer was separated, dried over Na₂SO₄, filtered and evaporated to give the title compound (3.2 g, 99%). ¹H NMR (500 MHz, CDCl₃) 1.36 (3H, t, J7.2); 4.31 (2H, q, J7.2); 6.54 (1H, d, J16.0); 7.97 (1H, d, J 16.0); 8.11 (1H, d, J2.2); 8.65 (1H, d, J2.2).

### Description 30

### Ethyl 3-[5-(trifluoromethyl)pyridin-3-yl]propanoate

To a nitrogen flushed suspension of Description 29 (3.21 g; 11.49 mmol) and triethylamine (1.76 ml; 12.6 mmol) in methanol (100 ml), was added 10% palladium on carbon (0.5 g), and the resulting mixture stirred under a balloon of hydrogen overnight. The catalyst was removed by filtration and the filtrate evaporated to give a white solid (2.6 g, 91%). ¹H NMR (500 MHz, CDCl₃) δ 1.23 (3H, t, J7.1); 2.68 (2H, t, J7.4); 3.04 (2H, t, J7.4); 4.13 (2H, q, J7.1); 7.78 (1H, s); 8.68 (1H, s); 8.75 (1H, s).

### Description 31

### 3-[5-(Trifluoromethyl)pyridin-3-yl]propanoic acid

To a mixture of Description 30 (2.59 g; 10.5 mmol) in ethanol (25 ml) was added a solution of sodium hydroxide (2.1 g; 52.5 mmol) in water (75 ml) and the resulting mixture heated at reflux for 1 hour. The mixture was cooled and evaporated. The residue was dissolved in water and extracted with diethyl ether (×2). The aqueous was acidified by the addition of 2N HCl and extracted with DCM (×2), combined DCM layers washed with sat. NaCl, dried over Na₂SO₄, filtered and evaporated to give a white solid (2.0 g, 86%). ¹H NMR (500 MHz, CDCl₃) δ 2.74 (2H, t, J7.3); 3.07 (2H, t, J7.3); 7.85 (1H, s); 8.72 (1H, s); 8.75 (1H, s).

### Description 32

### Ethyl 3-azido-4-hydroxycyclohexanecarboxylate

To a solution of ethyl-7-oxabicyclo[4.1.0]heptane-3-carboxylate (EP 520419 A2 (1992); 10 g, 58.75 mmol) in anhydrous DMF (75 ml) was added successively ammonium chloride (4.54 g; 88.13 mmol) and sodium azide (5.73 g; 88.15 mmol), and the resulting mixture heated at 75° C. overnight. The mixture was cooled and evaporated to about 1/3 volume and partitioned between water (150 ml) and EtOAc (100 ml). The organic layer was washed with water (100 ml), brine (50 ml), dried over Na₂SO₄, filtered and evaporated. The residue was purified by column chromatography on silica (eluent: 20% EtOAc in hexanes) to give the title compound (11.49 g, 91%). ¹H NMR (400 MHz, CDCl₃) 1.25 (3H, t, J7.1); 1.45-1.63 (2H, m); 1.87-1.93 (1H, m); 2.06-2.13 (1H, m); 2.33-2.39 (2H, m); 2.72 (1H, quintet, J4.7); 3.47-3.58 (2H, m); 4.17 (2H, q, J7.1).

### Description 33

### Ethyl 3-amino-4-hydroxycyclohexanecarboxylate

To a nitrogen flushed solution of Description 32 (11.49 g; 53.9 mmol) in EtOAc (150 ml) was added 10% Palladium on carbon (1.5 g), and the resulting mixture stirred under a balloon of hydrogen overnight. The catalyst was removed by filtration and the mixture charged with fresh 10% Palladium on carbon (1.5 g), and hydrogenated at 30 psi in a PARR apparatus for 3 hours. The catalyst was removed by filtration and the filtrate evaporated to give the title compound (10 g; 99%).

### Description 34

### Ethyl 4-hydroxy-3-[(trifluoroacetyl)amino]cyclohexane carboxylate

To a mixture of Description 33 (10.09 g; 53.9 mmol, and triethylamine (15.02 ml, 107.8 mmol) in anhydrous dichloromethane (100 ml) cooled in an ice bath was added dropwise trifluoroacetic anhydride (8.74 ml, 61.99 mmol). The resulting mixture was stirred at room temperature for 2 hours then evaporated. The residue was partitioned between EtOAc (200 ml) and 1M Citric acid solution (200 ml). The organic layer was washed with sat. aqueous NaHCO₃ (100 ml), brine (100 ml), dried over Na₂SO₄, filtered and evaporated to give the title compound (15.2 g, quant).

### Description 35

### Ethyl 4-oxo-3-[(trifluoroacetyl)amino]cyclohexane carboxylate

To a solution of Description 34 (15.27 g; 53.9 mmol) in anhydrous dichloromethane (300 ml) was added Dess Martin periodinane (27.43 g; 64.68 mmol) and the resulting mixture stirred for 4 hours. Further Dess Martin periodinane (10 g; 23.6 mmol) was added and stirring continued overnight. The mixture was evaporated and the residue purified by column chromatography on silica (eluent: 25% EtOAc in hexanes). The product was dissolved in dichloromethane (150 ml) and washed with sat aqueous NaHCO₃ (150 ml), dried over Na₂SO₄, filtered and evaporated to give a yellow oil which crystallised on standing (11.2 g, 74%).

### Description 36

### Ethyl 2-(trifluoromethyl)-4,5,6,7-tetrahydro-1,3-benzothiazole-5-carboxylate

To a solution of Description 35 (5.00 g; 17.78 mmol) in anhydrous toluene (200 ml) was added Lawesson's Reagent (14.38 g; 35.56 mmol) and the resulting mixture heated at reflux for 15 hours. The mixture was cooled and concentrated to a volume of about 50 ml and loaded directly onto a silica gel column (eluent: 10% EtOAc in hexanes). The product was further purified by column chromatography on silica (eluent: 10% Et₂O in hexanes) to give a yellow oil (1.46 g, 29%). ¹H NMR (500 MHz, CDCl₃) 1.27 (3H, t, J7.1); 1.98-2.06 (1H, m); 2.27-2.31 (1H, m); 2.83-2.91 (2H, m); 2.97-3.20 (3H, m); 4.20 (2H, q, J7.1).

### Description 37

### 2-(Trifluoromethyl)-4,5,6,7-tetrahydro-1,3-benzothiazole-5-carboxylic acid

To a solution of Description 36 (1.46 g; 5.22 mmol) in ethanol (10 ml) was added a solution of sodium hydroxide (420 mg; 10.44 mmol) in water (20 ml) and the resulting mixture warmed to 80° C. for 30 minutes. The ethanol was removed by evaporation and the aqueous acidified by the addition of 2N HCl. The mixture was extracted with DCM (3×30 ml), combined DCM layers dried over Na₂SO₄, filtered and evaporated to give a white solid (770 mg, 58%). ¹H NMR (500 MHz, CDCl₃) 2.03-2.14 (1H, m); 2.31-2.37 (1H, m); 2.87-2.98 (2H, m); 2.98-3.07 (1H, m); 3.07-3.18 (1H, m); 3.21 (1H, dd, J16.8 and 5.7).

### Description 38

### 5-Amino-N-(4-chlorophenyl)-1-ethyl-1H-imidazole-4-carboxamide

Prepared from Description 1 according to the method of Description 16. ¹H NMR (500 MHz, DMSO) δ 9.44 (1H, s), 7.83 (2H, d, J8.9), 7.29 (2H, d, J8.9), 7.24 (1H, s), 6.02 (2H, s), 3.85 (2H, q, J7.3), 1.27 (3H, t, J7.2).

### Description 39

### N-(4-Chlorophenyl)-5-[(3-cyclopropylpropanoyl)amino]-1-ethyl-1H-imidazole-4-carboxamide

Description 38 (300 mg, 1.14 mmol), 3-cyclopropylpropanoic acid (220 mg, 1.93 mmol), bis(2-oxo-3-oxazolidinyl)phosphinic chloride (490 mg, 1.93 mmol), N,N-diisopropylethylamine (335 μl, 1.93 mmol) and dichloroethane (10 ml) were combined and heated in a microwave at 160° C. for 20 minutes, after which time TLC indicated the reaction was complete. The mixture was partitioned between dichloromethane (30 ml) and 10% aqueous potassium carbonate (30 ml). The layers were separated, the aqueous phase extracted with more dichloromethane (30 ml) and the organic layers were combined and washed with water (30 ml) and 10% aqueous citric acid (30 ml). The organic phase was dried (MgSO₄), and evaporated. The residue was purified by mass directed preparative HPLC to give the title compound (145 mg, 35%). ¹H NMR (500 MHz, DMSO) δ 9.92 (1H, s), 9.81 (1H, s), 7.85 (2H, d, J9), 7.81 (1H, s), 7.34 (2H, d, J9), 3.82 (2H, q, J7), 2.44 (2H, t, J7), 1.52 (2H, q, J7), 1.30 (3H, t, J7), 0.80-0.72 (1H, m), 0.41 (2H, m), 0.08 (2H, m); m/z (ES⁺) 361 (M+H⁺).

### Description 40

### N-(4-Chlorophenyl)-1-methyl-5-{[(4-methyl-1,2,5-oxadiazol-3-yl)acetyl]amino}-1H-imidazole-4-carboxamide

Prepared from Description 16 and (4-methyl-1,2,5-oxadiazol-3-yl)acetic acid according to the procedure of Description 39. m/z (ES⁺) 375, 377 (M+H⁺).

### Description 41

### 2-(Trifluoromethyl)-1,3-thiazole-4-carbaldehyde

Diisobutyl aluminium hydride (1M in dichloromethane, 16.7 ml, 16.7 mmol) was added dropwise to a solution of Description 26 (1.88 g, 8.37 mmol) in anhydrous dichloromethane (10 ml) at −78° C. The reaction was stirred at −78° C. for 1 hr, and then excess aq. 2N hydrochloric acid was added and the reaction allowed to warm to room temperature. The reaction was extracted into ethyl acetate (×5) and the organic layer washed with water and saturated aqueous NH₄Cl, then dried (MgSO₄) and evaporated. Purification by flash column chromatography on silica (eluent: 25% ethyl acetate in hexane) gave the title compound as a pale yellow solid (650 mg, 43%) ¹H NMR (400 MHz, DMSO) δ 10.01 (1H, s), 9.07 (1H, s). (Additionally, some of the corresponding alcohol (19%) was isolated).

### Description 42

### Ethyl (2E)-2-methyl-3-[2-(trifluoromethyl)-1,3-thiazol-4-yl]acrylate

Description 41 (639 mg, 3.53 mmol) was added to a solution of ethyl 2-diethoxyphosphoryl)propanoate (0.980 ml, 4.6 mmol), lithium chloride (2.10 g, 49.4 mmol) and N-ethyl-N-isopropylpropan-2-amine (1.10 ml, 8.83 mmol) in anhydrous acetonitrile (10 ml) and the mixture stirred at room temperature for 24 hr. Aqueous ammonium chloride (120 ml) and water (50 ml) were added and the reaction extracted into dichloromethane (×5), then the combined organic phases were dried (MgSO₄) and evaporated. The crude material was purified by flash column chromatography on silica (eluent: 13% ethyl acetate in hexane) to give the title compound as a white solid (875 mg, 94%). ¹H NMR (400 MHz, DMSO) δ 8.48 (1H, s), 7.63 (1H, d, J1.2), 4.23 (2H, q, J7.1), 2.28 (3H, d, J1.2), 1.29 (3H, t, J7.1).

### Description 43

### Ethyl 2-methyl-3-[2-(trifluoromethyl)-1,3-thiazol-4-yl]propanoate

A mixture of Description 42 (818 mg, 3.10 mmol) and 5% palladium on carbon (300 mg) in ethanol (20 ml) was hydrogenated under a balloon of hydrogen gas at room temperature for 24 hr. The reaction mixture was filtered and evaporated to give the title compound as a colourless oil (825 mg, 100%). M/z (ES⁺) 268 (M+H⁺).

### Description 44

### 2-Methyl-3-[2-(trifluoromethyl)-1,3-thiazol-4-yl]propanoic acid

A mixture of Description 43 (825 mg, 3.1 mmol) and lithium hydroxide (147 mg, 6.2 mmol), in THF (8 ml) and water (2 ml) was stirred at room temperature for 16 hr. The reaction was acidified to pH 1 using conc. hydrochloric acid before extracting into ethyl acetate (×3). The organic layer was dried (MgSO₄) and evaporated to give the title compound as a yellow solid (691 mg, 94%). ¹H NMR δ (400 MHz, DMSO) δ 12.15 (1H, s), 7.83 (1H, s), 3.11 (1H, dd, J6.7, 14.0), 2.88-2.76 (2H, m), 1.09 (3H, d, J6.8).

### Description 45

### 5,5,5-Trifluoro-2-methylpentanoic acid

Lithium diisopropylamide (1.8 M in heptane/THF, 10.4 ml, 18.8 mmol) was added dropwise to a solution of 5,5,5-trifluoropentanoic acid (837 mg, 5.4 mmol) in anhydrous THF at 0° C. over 5 mins. The reaction was stirred at 0° C. for 15 mins, and then methyl iodide (1 ml, 16.2 mmol) was added dropwise; the resulting solution was allowed to warm to room temperature and stir for 16 hr. The reaction was acidified to pH 1 with conc. hydrochloric acid, extracted into ethyl acetate (×3) then the combined organic layers were washed with water/brine, then dried (MgSO₄) and evaporated to give the title compound as an orange oil (560 mg, 61%). ¹H NMR (500 MHz, DMSO) δ 12.40 (1H, bs), 2.44 (1H, q, J6.6), 2.28-2.17 (2H, m), 1.78-1.71 (1H, m), 1.58-1.52 (1H, m), 1.10 (3H, t, J6.6).

### Description 46

### 5-Fluoroindan-1-ol

A solution of 5-fluoro-1-indanone (2.0 g, 13.32 mmol) in 30 mL of methanol and 15 mL of dichloromethane was cooled to 0° C. in an ice bath, and sodium borohydride (504 mg, 13.32 mmol) was added portionwise. After the addition, the reaction mixture was stirred for 15 min at 0° C., followed by 30 min at room temperature. The reaction mixture was diluted with water and extracted twice with ether. The combined ether extracts were dried (Na₂SO₄), filtered, and evaporated in vacuo to give the title compound as a pale yellow oil (2.03 g, 100%). ¹H NMR (400 MHz, CDCl₃) δ 7.35 (1H, m), 6.92 (2H, m), 5.20 (1H, q, J6.4), 3.05 (1H, m), 2.80 (1H, m), 2.50 (1H, m), 1.99 (1H, m), 1.75 (1H, d, J7.2).

### Description 47

### 1-Chloro-5-fluoroindane

A mixture of 5-fluoroindan-1-ol (2.03 g, 13.32 mmol) and thionyl chloride (1.5 mL, 19.98 mmol) in 20 mL of toluene was stirred for 30 min at room temperature, followed by 17 h at 55° C. After cooling to room temperature, the solvents were evaporated to give a brown oil. The oil was taken up in ethyl acetate (25 mL) and washed with water (20 mL) and saturated NaHCO₃ (20 mL). The ethyl acetate layer was dried (Na₂SO₄), filtered, and evaporated in vacuo to give the title compound as a brown oil (1.9 g, 84%). ¹H NMR (400 MHz, CDCl₃) δ 7.37 (1H, m), 6.92 (2H, m), 5.40 (1H, m), 3.20 (1H, m), 2.90 (1H, m), 2.62 (1H, m), 2.41 (1H, m).

### Description 48

### 1-Cyano-5-fluoroindane

A solution of 1-chloro-5-fluoroindane (1.9 g, 11.1 mmol) in 50 mL of DMF was treated with sodium cyanide (870 mg, 17.76 mmol), and the resulting reaction mixture was heated to 70° C. and stirred for 21 h. An additional portion of sodium cyanide (500 mg) was added after the first 8 h. After cooling to room temperature, the reaction mixture was poured into ice water (200 mL), and the resulting mixture was extracted with CH₂Cl₂ (3×60 mL). The combined CH₂Cl₂ extracts were washed with brine (60 mL), dried (Na₂SO₄), filtered, and evaporated to give a black liquid. Purification by column chromatography (gradient from hexane to 20% ethyl acetate/hexane) afforded the title compound as yellow-brown oil (780 mg, 43%). ¹H NMR (400 MHz, CDCl₃) δ7.37 (1H, m), 6.96 (2H, m), 4.10 (1H, m), 3.10 (1H, m), 2.95 (1H, m), 2.60 (1H, m), 2.41 (1H, m).

### Description 49

### 5-Fluoro-1-indancarboxylic acid

A mixture of 1-cyano-5-fluoroindane (200 mg, 1.24 mmol) and 50% aqueous KOH (4.0 mL) in 12 mL of ethanol was heated to reflux for 14 h. After cooling to room temperature, the solution was extracted with ether (10 mL). The aqueous extract was diluted with water (10 mL) and cooled to 0° C. Concentrated HCl was carefully added to adjust the pH to 2-3. The resulting cloudy mixture was extracted with CH₂Cl₂ (20 mL), and the CH₂Cl₂ layer was dried (Na₂SO₄), filtered, and evaporated in vacuo to give the title compound as a brown oil, which slowly solidified on standing (212 mg, 95%). ¹H NMR (400 MHz, CDCl₃) δ 7.35 (1H, m), 6.90 (2H, m), 4.03 (1H, t, J7), 3.10 (1H, m), 2.90 (1H, m), 2.4 (2H, m).

### Description 50

### 1,2-Bis(bromomethyl)-4-(trifluoromethyl)benzene

A mixture of 3,4-dimethylbenzotrifluoride (5 g, 28.7 mmol), N-bromosuccinimide (11.2 g, 63.14 mmol), and a catalytic amount of benzoyl peroxide (50 mg) in 45 mL of CCl₄ was heated to reflux for 8 h. After cooling to room temp, the reaction mixture was filtered and the filtrate concentrated in vacuo. The resulting pale yellow oil was purified by column chromatography (hexane) to give the title compound as a clear oil (4.9 g, 52%). ¹H NMR (400 MHz, CDCl₃) δ 7.63 (1H, s), 7.57 (1H, d, J8), 7.50 (1H, d, J8), 4.66 (2H, s), 4.65 (2H, s).

### Description 51

### Diethyl 5-(trifluoromethyl)-1,3-dihydro-2H-indene-2,2-dicarboxylate

Sodium metal (713 mg, 31.0 mmol) was added to 10 mL of absolute ethanol in a 250 mL round bottom flask and the mixture was stirred until all the sodium dissolved. Anhydrous diethyl ether (28 mL) was then added, followed by diethyl malonate (2.24 mL, 14.76 mmol). To this solution was added, as quickly as possible, 1,2-bis(bromomethyl)-4-(trifluoromethyl)benzene (4.9 g, 14.76 mmol) in 28 mL of ether. The reaction was then heated to reflux and stirred for 1 h. After cooling to room temp, the reaction mixture was filtered and the filtrate concentrated in vacuo. The dark blue oil was purified by column chromatography (10% ethyl acetate/hexanes) to give the title compound as a pale yellow oil (3.38 g, 69%). ¹H NMR (400 z, CDCl₃) δ 7.43 (2H, m), 7.30 (1H, d, J8), 4.2 (4H, q, J7.6, 6.8), 3.63 (4H, s), 1.26 (6H, t, J7.2).

### Description 52

### 5-Trifluoromethyl 1,3-dihydro-2H-indene-2,2-dicarboxylic acid

A mixture of diethyl 5-trifluoromethyl)-1,3-dihydro-2H-indene-2,2-dicarboxylate (3.38 g, 10.2 mmol) and KOH (2.5 g) in 15 mL of water and 10 mL of ethanol was heated to reflux for 3 h. After cooling to room temp, the ethanol was removed in vacuo, and the aqueous solution was acidified to pH=2-3 with 6.0 N aq. HCl. The resulting precipitate was filtered and dried in vacuo to give the title compound as a light brown solid (2.8 g, 100%). ¹H NMR (400 MHz, CDCl₃) δ 7.53 (1H, s), 7.46 (1H, d, J8), 7.38 (1H, d, J 8), 3.42 (4H, s).

### Description 53

### 5-(Trifluoromethyl)indane-2-carboxylic acid

5-(Trifluoromethyl)-1,3-dihydro-2H-indene-2,2-dicarboxylic acid (500 mg, 1.82 mmol) was placed in a small round bottom flask and heated to 200° C. for 20 minutes. The solid turned dark brown between 185-195° C., and fumes could be seen coming from the flask. After cooling to room temp, the residue was dissolved in 10 mL of 1.0 N aq. NaOH and extracted with ethyl acetate (10 mL). The aqueous layer was acidified to pH=2-3 with 6.0 N aq. HCl, causing a brown oil to separate from solution. The mixture was extracted with CH₂Cl₂ (25-30 mL), and the CH₂Cl₂ extract was dried (Na₂SO₄), filtered, and evaporated in vacuo to give the title compound as a brown oil, which slowly solidified on standing (270 mg, 64%). ¹H NMR (40 MHz, CDCl₃) δ 10.9 (1H, bs), 7.44 (2H, m), 7.31 (1H, d, J8), 3.3-3.5 (5H, m).

### Description 54

### Ethyl 1-methyl-5-{[(2,4,6-trifluorophenyl)acetyl]amino}-1H-imidazole-4-carboxylate

A mixture of ethyl 5-amino-1-methyl-1H-imidazole-4-carboxylate (2.2 g, 13.0 mmol) and 2,4,6-trifluorophenylacetic acid (2.5 g, 13.0 mmol) in 18 mL of trimethylacetic anhydride was heated to 85° C. and stirred for 23 h. The mixture was cooled to room temperature and poured into diethyl ether (50 mL). The solid precipitate that formed was filtered and dried in vacuo to provide the title compound as a white solid (2.7 g, 61%). ¹H NMR (400 MHz, CDCl₃) δ 8.46 (1H, bs), 7.36 (1H, s), 6.74 (2H, t, J8), 4.35 (2H, q, J6.8, 7.6), 3.83 (2H, s), 3.61 (3H, s) 1.39 (3H, t, J7.2).

### Description 55

### 3-Methyl-5-(2,4,6-trifluorobenzyl)imidazo[4,5-d][1,3]oxazin-7(3H)-one

A suspension of ethyl 1-methyl-5-{[(2,4,6-trifluorophenyl)acetyl]amino}-1H-imidazole-4-carboxylate (2.7 g, 7.91 mmol) in 25 mL of toluene was treated with phosphorus oxychloride (1.4 mL, 15.82 mmol). The reaction mixture was heated to reflux for 21 h. After cooling to room temperature, the solvent was removed in vacuo to give a brown residue, which was taken up in ethyl acetate (40 mL) and washed with 20 mL of saturated NaHCO₃. The ethyl acetate layer was dried (Na₂SO₄), filtered, and evaporated in vacuo to give a dark brown oil. Purification by column chromatography (5% MeOH/CH₂Cl₂) afforded the title compound as a light brown solid (1.6 g, 68%). ¹H NMR (400 MHz, CDCl₃) δ 7.67 (1H, s), 6.72 (2H, t, J8), 4.08 (2H, s), 3.69 (3H, s).

### Description 56

### N-(5-Chloro-1,3-thiazol-2-yl)-1-methyl-5-{[(2,4,6-trifluorophenyl)acetyl]amino}-1H-imidazole-4-carboxamide

A suspension of 2-amino-5-chlorothiazole hydrochloride (174 mg, 1.02 mmol) in 15 mL of ethyl acetate was treated with 1 mL of saturated NaHCO₃. The layers were separated and the ethyl acetate layer concentrated in vacuo to give the free base as a light brown solid. A mixture of this free base, 3-methyl-5-(2,4,6-trifluorobenzyl)imidazo[4,5-d][1,3]oxazin-7(3H)-one (200 mg, 0.68 mmol), and pivalic acid (347 mg, 3.4 mmol) was dissolved in CH₂Cl₂ and concentrated to give a thick brown oil, which was heated to 160° C. for 10 min After cooling to room temperature, the black residue was dissolved in 5% MeOH/CH₂Cl₂ and washed with water and saturated NaHCO₃. The organic extract was dried (Na₂SO₄), filtered, and evaporated to provide the title compound as a dark brown solid residue, which was used without further purification (250 mg, 86%). ¹H NMR (400 MHz, CDCl₃) δ 10.02 (1H, bs), 8.57 (1H, bs), 7.69 (1H, d, J9.8), 7.36 (1H, s), 6.72 (2H, m), 3.98 (1H, s), 3.65 (3H, s), 1.24 (1H, s).

### Description 57

### 4-(Trimethylsilyloxy)chroman-4-carbonitrile

Chroman-4-one (4.44 g, 0.03 moles) and zinc iodide (100 mg) were dissolved in CH₂Cl₂ at room temp under nitrogen atmosphere. To this mixture was added drop wise trimethylsilylcyanide (3.2 g, 0.0315 moles) and stirred at room temp for two days. Added additional trimethylsilylcyanide (2.2 g) to the reaction mixture and refluxed for 4 hours. The reaction mixture was concentrated in vacuo and the residue was purified by column chromatography using 15% ethyl acetate/hexane to afford the title compound as yellow viscous oil (7.2 g, 96% yield). ¹H NMR (400 MHz, CDCl₃) 7.6 (1H, d), 7.25 (1H, t), 7.0 (1H, t), 6.85 (1H, d), 4.35 (2H, m), 2.4 (2H, m), 0.2 (9H, s).

### Description 58

### Chroman-4-carboxylic acid

4-Trimethylsilyloxy)chroman-4-carbonitrile (7.2 g, 0.029 moles) and tin (II) chloride (23.6 g, 0.125 moles) were dissolved in glacial AcOH (30 mL) and con. HCl (30 mL). The resultant mixture was heated at 140° C. for 20 hours and then cooled to room temp. Diluted the mixture with water (150 mL), extracted with CH₂Cl₂ (3×150 mL), washed the combined extracts with brine and dried (MgSO₄). The dried extract was filtered and concentrated under vacuo to afford the title compound as off-white solid (4.45 g. 87% yield). ¹H NMR (400 MHz, CDCl₃) 7.26 (1H, m), 7.16 (1H, m), 6.84 (1H, m), 4.25 (2H, m), 3.81 (1H, m), 2.32 (1H, m), 2.13 (1H, m).

### Description 59

### ethyl 5-amino-1-(2,2-difluoroethyl)-1H-imidazole-4-carboxylate

Prepared according to the method of Description 1, using 2,2-difluoroethylamine instead of ethylamine.

### Description 60

### 5-amino-N-(4-chlorophenyl)-1-(2,2-difluoroethyl)-1H-imidazole-4-carboxamide

Prepared from Description 59 and 4-chloroaniline according to the procedure of Description 16.

### Description 61

### ethyl 2-(4,4-difluorocyclohexyl)acetate

[Bis(2-methoxyethyl)amino]sulfur trifluoride (Deoxofluor®, 7.5 g, 34.8 mmol) was added to a solution of ethyl 2-(4-oxocyclohexyl)acetate [Alonso F., et. al. Tetrahedron, 1995, 51, 10259-10280] (2.0 g, 10.9 mmol) in dichloromethane (20 mL). The mixture was stirred for 3 days at room temperature and quenched with sat. NaHCO₃ (20 mL) and extracted with ethyl acetate (3×100 mL) and the combined organic phases were dried (MgSO₄) and evaporated. The residue was purified with flash chromatography to give the title compound.

### Description 62

### 2-(4,4-difluorocyclohexyl)acetic acid

KOH (2.1 g, 32 mmol) was added to a solution of (4,4-difluoro-cyclohexyl)-acetic acid ethyl ester (1.3 g, 6.3 mmol) in MeOH—H₂O (4:1, 50 mL). The mixture was stirred for 3 h at room temperature, concentrated, diluted with H₂O (20 mL), washed with ether (50 mL). The aqueous layer was acidified to pH=2 and extracted with ethyl acetate (3×500 mL) and the combined organic phases were dried (MgSO₄) and evaporated to give the title compound.

### Description 63

### 2-(3,4-difluorophenyl)-2,2-difluoroacetic acid

KOH (89 mg, 1.35 mmol) was added to a solution of (3,4-difluoro-phenyl)-difluoro-acetic acid ethyl ester [Middleton, W. J. & Bingham, E. M. J. Org. Chem., 1980, 45, 2883-7] (80 mg, 0.34 mmol) in MeOH—H₂O (4:1, 20 mL). The mixture was heated to reflux for 4 h. The mixture was cooled to room temperature, concentrated, diluted with H₂O (10 mL), washed with ether (20 mL). The aqueous layer was acidified to pH=2 and extracted with ethyl acetate (3×30 mL) and the combined organic phases were dried (MgSO₄) and evaporated to give the title compound.

### Description 64

### methyl 2-(3,4-difluorophenyl)-2-fluoroacetate

LiHMDS (1M in THF, 3.45 mL, 3.45 mmol) was added dropwise to a cooled solution (−78° C.) of (3,4-difluoro-phenyl)-acetic acid methyl ester (560 mg, 3.0 mmol) in THF (20 mL) and the mixture was stirred at −78° C. for 1 h. A solution of N-fluorobenzene-sulfonimide (1.2 g, 3.75 mmol) in THF (10 mL) was added dropwise. The mixture was stirred at −78° C. for 4 h and quenched with sat, NaHCO₃. The mixture was extracted with ethyl acetate (3×100 mL) and the combined organic phases were dried (MgSO₄) and evaporated. The residue was purified with flash chromatography to give the title compound.

### Description 65

### 2-(3,4-difluorophenyl)-2-fluoroacetic acid

KOH (145 mg, 2.2 mmol) was added to a solution of (3,4-difluoro-phenyl)-fluoro-acetic acid methyl ester (90 mg, 0.44 mmol) in MeOH—H₂O (4:1, 20 mL). The mixture was heated to reflux for 4 h. The mixture was cooled to room temperature, concentrated, diluted with H₂O (10 mL), washed with ether (20 mL). The aqueous layer was acidified to pH=2 and extracted with ethyl acetate (3×30 mL) and the combined organic phases were dried (MgSO₄) and evaporated to give the title compound.

### Description 66

### 4,4,4-trifluoro-3-methylbutyl methanesulfonate

Methanesulfonyl chloride (1.64 mL, 21.1 mmol) and TEA (3.9 mL, 28.2 mmol) were added dropwise to a cooled solution of 4,4,4-trifluoro-3-methyl-butan-1-ol [EP 0300497] (2.0 g, 14.1 mmol) in THF (40 mL). The mixture was stirred at room temperature for 2 h and quenched with saturated NaHCO₃. The mixture was extracted with ether (3×50 mL) and the combined organic phases were dried (MgSO₄) and evaporated to give the title compound.

### Description 67

### 5,5,5-trifluoro-4-methylpentanenitrile

NaCN (2.1 g, 42.3 mmol) was added to a solution of methanesulfonic acid 4,4,4-trifluoro-3-methyl-butyl ester (3.1 g, 14.1 mmol) in DMSO (40 mL). The mixture was heated to 120° C. for 24 h. The mixture was cooled to room temperature, diluted with ether (200 mL), washed with water (3×40 mL), the organic phases were dried (MgSO₄), and evaporated. The residue was purified with flash chromatography (25% EtOAc-Hexanes) to give the title compound.

### Description 68

### 5,5,5-trifluoro-4-methylpentanoic acid

25% NaOH (20 mL) was added to a solution of 5,5,5-trifluoro-4-methyl-pentanenitrile (1.2 g, 7.9 mmol) in EtOH (20 mL). The mixture was heated to reflux for 24 h. The mixture was cooled to room temperature, concentrated, diluted with H₂O (10 mL), washed with ether (20 mL). The aqueous layer was acidified to pH=2 and extracted with ether (3×30 mL) and the combined organic phases were dried (MgSO₄) and evaporated to give the title compound.

### Description 69

### 5-Amino-N-(4-chlorophenyl)-1-cyclopropyl-1H-imidazole-4-carboxamide

Prepared from Description 20 and 4-chloroaniline according to the procedure of Description 16. m/z (ES⁺) 277 (M+H⁺).

### Description 70

### N-(4-Chlorophenyl)-1-cyclopropyl-5-(1,2,3,4-tetrahydronaphthalene-3-carboxamido)-1H-imidazole-4-carboxamide

Prepared from Description 69 and 1,2,3,4-tetrahydronaphthalene-2-carboxylic acid according to the procedure of Description 39. m/z (ES⁺) 435 (M+H⁺).

### Description 71

### N-(4-chlorophenyl)-5-[(1,2,3,4-tetrahydronaphthalene-3-carboxamido)thiazole-4-carboxamide

Prepared from Description 18 and 1,2,3,4-tetrahydronaphthalene-2-carboxylic acid according to the procedure of Description 39. m/z (ES⁺) 413 (M+H⁺).

### Description 72

### N-(4-Chlorophenyl)1-ethyl-5-(2-(2-methylthiazol-4-yl)-butanamido]-1H-imidazole-4-carboxamide

Prepared from Description 77 and 2-(2-methyl-thiazol-4-yl)-butyric acid according to the procedure of Description 39. m/z (ES⁺) 432 (M+H⁺.

### Description 73

### 2-(2-Ethylthiazol-4-yl)acetic acid

Ethyl chloroacetate (1 equiv) was dissolved in dry acetone, and a solution of thiopropanamide (1 equiv) in acetone was added. The reaction was refluxed overnight. After cooling to rt, volatiles were evaporated. The residue was diluted with ethyl acetate followed by careful addition of saturated NaHCO₃ (aq). Layers were separated and the aqueous layer was extracted with ethyl acetate (2×), organics were combined, dried (Na₂SO₄) and concentrated to give a yellow residue. Purification by column chromatography on silica gel (ethyl acetate:hexanes: 80:20) provided (2-Ethyl-thiazol-4-yl)-acetic acid ethyl ester in 45% yield. m/z (ES⁺) 200 (M+H⁺).

2-Ethyl-thiazol-4-yl)-acetic acid ethyl ester (1 equiv) was dissolved in ethanol and 4N NaOH (aq) (5 equiv) was added. The reaction was left to stir overnight at rt. The ethanol was evaporated, the residue acidified to pH 4, and extracted with ethyl acetate (4×). The organic extracts were combined, dried (Na₂SO₄) and concentrated to give a yellow solid. The solid was stirred for 3 h in a solution of ether:hexanes (1:3). After filtration and air drying, the acid was obtained as a beige solid in 93% yield. m/z (ES⁺) 172 (M+H⁺).

### Description 74

### Ethyl 1-Ethyl-5-(2-(3-trifluoromethylphenyl)acetamido)-1H-imidazole-4-carboxylate

A mixture of Description 1 (0.2 g, 1.09 mmol), (3-trifluoromethyl-phenyl)-acetyl chloride (0.36 g, 1.5 equiv), diisopropylethylamine (0.35 g, 2.5 equiv) in THF (5 mL) was heated at 80° C. for 26 h. Volatiles were evaporated from the cooled reaction and the residue was taken into CH₂Cl₂ and H₂O. The organic layer was dried (Na₂SO₄), filtered and concentrated. Purification by Preparative TLC provided the title compound in 65% yield. m/z (ES⁺) 370 (M+H⁺).

### Description 75

### 5-(3-trifluoromethylbenzyl)-3-ethylimidazo[4,5-d][1,3]oxazin-7(3H)-one

Prepared from Description 74 according to the procedure of Description 8.

### Description 76

### N-(4-Chlorophenyl)-1-ethyl-5-(2-(3-trifluoromethyl)phenylacetamido)-1H-imidazole-4-carboxamide

Prepared from Description 77 and trifluoromethyl acetic acid according to the procedure of Description 8.

### Description 77

### 5-Amino-1-N-(4-chlorophenyl)-1-ethyl-1H-imidazole-4-carboxamide

Prepared from Description 1 and 4-chloroaniline according to procedure of Description 16.

### EXAMPLE 1

### 1-(4-Chlorophenyl)-2-[2-(3-fluorophenyl)ethyl]-9-methyl-1,9-dihydro-6H-purin-6-one

A catalyst mixture of copper(I)iodide and bis(triphenylphosphino)palladium(II)dichloride (1:1 molar ratio; 5 mg) was added to a mixture of Description 6 (110 mg, 0.373 mmol), 1-ethynyl-3-fluorobenzene (80 μL, 0.693 mmol) and triethylamine (250 μL) in N,N-dimethylacetamide (4 ml). The reaction was heated in a microwave reactor at 110° C. for 20 minutes. More catalyst mixture (5 mg) was added and the reaction heated for a further 20 minutes at 110° C., then 20 minutes at 130° C. More alkyne (40 μL, 0.347 mmol) and more catalyst (5 mg) were added, the reaction heated at 130° C. for a further 20 minutes, then the reaction mixture was evaporated. The residue was purified by preparative thin layer chromatography (eluant: 15% MeOH in ethyl acetate) and the product then triturated with methanol and collected by filtration to give 1-(4-chlorophenyl)-2-[(3-fluorophenyl)ethynyl]-9-methyl-1,9-dihydro-6H-purin-6-one (35 mg). m/z (ES⁺) 379, 381 (M+H⁺). A sample of 1-(4-chlorophenyl)-2-[(3-fluorophenyl)ethynyl]-9-methyl-1,9-dihydro-6H-purin-6-one (30 mg) was dissolved in methanol-ethyl acetate (1:1; 5 ml). Palladium on carbon (50 mg) was added and the reaction stirred under a balloon of hydrogen gas for 1 h. The reaction was filtered, the filtrate evaporated and the residue was purified by preparative thin layer chromatography (eluant: 10% MeOH in ethyl acetate) to give the title compound (8 mg). ¹H NMR (400 MHz, CDCl₃) 7.73 (1H, s), 7.49 (2H, d, J8.4), 7.24-7.16 (1H, app. q, J7), 7.06 (2H, d, J8.5), 6.90-6.72 (3H, m), 3.84 (3H, s), 3.05 (2H, t, J7.5), 2.69 (2H, t, J7.5); m/z (ES⁺) 383, 385 (M+H⁺).

### EXAMPLE 2

### 1-(4-Chlorophenyl)-9-ethyl-2-[3-(trifluoromethyl)phenyl]-1,9-dihydro-6H-purin-6-one

A suspension of Description 3 (75 mg, 0.24 mmol), Pd (dppf)Cl₂ (10 mg, 0.012 mmol), [3-(trifluoromethyl)phenyl]boronic acid (45 mg, 0.24 mmol), and a saturated aqueous solution of sodium carbonate (250 μl) in tetrahydrofuran (2 ml) was irradiated in a Smith Synthesizer microwave at 150° C. for 10 minutes. The resulting solution was partitioned between dichloromethane (5 ml) and water (5 ml) and the layers separated. The resulting organic solution was then passed through a SCX (strong cation exchange) cartridge, washing with dichloromethane and methanol then eluting with 2M ammonia in methanol. Evaporation of the basic fraction gave a pale purple solid which was purified by mass directed HPLC to give the title compound as a white solid (22 mg, 22%). ¹H NMR (400 MHz, DMSO) δ 8.26 (1H, s), 7.72-7.67 (3H, m), 7.52 (1H, t, J7.8), 7.40-7.36 (4H, m), 4.24 (2H, q, J7.2), 1.44 (3H, t, J7.3); m/z (ES⁺) 419, 421 (M+H⁺).

### EXAMPLE 3

### 1-(4-Chlorophenyl)-9-methyl-2-(4,4,4-trifluorobutyl)-1,9-dihydro-6H-purin-6-one

### Method 1

Phosphorus oxychloride (93 μL, 0.995 mmol) was added to a suspension of Description 14 (193 mg, 0.497 mmol) in toluene (8 ml) and the reaction mixture heated at reflux for 4 h, after which time tlc indicated complete reaction. The reaction was cooled to room temperature, the toluene evaporated and the mixture was partitioned between dichloromethane (10 ml) and 10% aqueous potassium carbonate solution (30 ml). The layers were separated, the aqueous phase extracted with more dichloromethane (20 ml) and the combined organic phases were dried (Na₂SO₄) and evaporated. The residue was triturated with ether and the solid collected by filtration and dried in vacuo to give the title compound (130 mg, 71%). ¹H NMR (400 MHz, CDCl₃) 7.75 (1H, s), 7.53 (2H, d, J8), 7.16-7.14 (2H, d, J8), 3.83 (3H, s), 2.46 (2H, t, J7.1), 2.21-1.99 (4H, m); m/z (ES⁺) 371, 373 (M+H⁺).

### Method 2

Description 16 (50 mg, 0.20 mmol) was combined with 5,5,5-trifluoropentanoic acid (62 mg, 0.40 mmol) and polyphosphoric acid (0.5 ml) in a sealed tube. The mixture was heated to 150° C. and stirred for 20 mins. On cooling, water was added and the polyphosphoric acid plug broken up and the suspension partitioned between 4N aqueous NaOH (20 ml) and CH₂Cl₂ (20 ml). The aqueous phase was extracted with further CH₂Cl₂ (20 ml) and the combined organic fractions dried over MgSO₄, filtered and concentrated in vacuo. The resulting yellow oil was triturated with Et₂O to afford the title compound as an ivory solid (66 mg, 88%). Data was consistent with that for the material produced by Method 1 above.

### EXAMPLE 4

### 1-(4-Fluorophenyl)-9-methyl-2-(4,4,4-trifluorobutyl-1,9-dihydro-6H-purin-6-one

Prepared from Description 10 and 4-fluoroaniline according to the procedures of Description 14 and Example 3 respectively. ¹H NMR (400 MHz, CDCl₃) 7.75 (1H, s), 7.25-7.15 (4H, m), 3.83 (3H, s), 2.46 (2H, t, J7.1), 2.21-1.99 (4H, m); m/z (ES⁺) 355 (M+H⁺).

### EXAMPLE 5

### 1-(4-Chlorophenyl)-9-methyl-2-(3,3,3-trifluoropropyl)-1,9-dihydro-6H-purin-6-one

Prepared from Description 8 and 4-chloroaniline according to the procedures of Description 14 and Example 3 respectively. ¹H NMR (500 MHz, CDCl₃) 7.74 (1H, s), 7.55 (2H, d, J8), 7.17 (2H, d, J8), 3.83 (3H, s), 2.73-2.58 (4H, m); m/z (ES⁺) 357, 359 (M+H⁺).

### EXAMPLE 6

### 1-(4-Chlorophenyl)-2-(2-cyclohexylethyl)-9-methyl-1,9-dihydro-6H-purin-6-one

Prepared from Description 11 and 4-chloroaniline according to the procedures of Description 14 and Example 3 respectively. ¹H NMR (400 MHz, CDCl₃) 8.05 (1H, s), 7.63 (2H, d, J8), 7.43 (2H, d, J8), 3.75 (3H, s), 2.35 (2H, t, J7.9), 1.60-1.43 (7H, m), 1.15-1.00 (4H, m), 0.77-0.66 (2H, m). m/z (ES⁺) 371, 373 (M+H⁺).

### EXAMPLE 7

### 1-(4-Chlorophenyl)-9-methyl-2-(3-phenylpropyl)-1,9-dihydro-6H-purin-6-one

Phosphorus oxychloride (105 μL, 1.2 mmol) was added to a suspension of Description 15 (150 mg, 0.4 mmol) in toluene (8 ml) and the reaction mixture heated at reflux for 4 h, after which time tlc indicated complete reaction. The reaction was cooled to room temperature, and the mixture was partitioned between ethyl acetate (15 ml) and sat aqueous potassium carbonate solution (15 ml). The layers were separated, the aqueous phase extracted with more ethyl acetate (15 ml) and the combined organic phases were dried (MgSO₄) and evaporated. The residue was triturated with ether and the solid collected by filtration and dried in vacuo to give the title compound (115 mg, 76%). ¹H NMR (400 MHZ, DMSO) δ 8.05 (1H, s), 7.58 (2H, d, J8.6), 7.38 (2H, d, J8.6), 7.24-7.20 (2H, m), 7.16-7.13 (1H, m), 7.07 (2H, d, J7.1), 3.76 (3H, s), 2.54 (2H, t, J7.4), 2.33 (2H, t, J7.4), 1.94-1.88 (2H, m); m/z (ES⁺) 379, 381 (M+H⁺).

### EXAMPLE 8

### 9-Methyl-1-phenyl-2-(4,4,4-trifluorobutyl)-1,9-dihydro-6H-purin-6-one

Prepared from Description 17 according to the procedure of Example 3 (Method 2). ¹H NMR (500 MHz, DMSO): δ 8.06 (1H, s), 7.58-7.50 (3H, m), 7.36 (2H, d, J7.3), 3.76 (3H, s), 2.41 (2H, t, J7.2), 2.32-2.22 (2H, m), 1.91-1.85 (2H, m). m/z (ES⁺) 336 (M+H⁺).

### EXAMPLE 9

### 6-(4-Chlorophenyl)-5-(4,4,4-trifluorobutyl)[1,3]thiazolo[5,4-d]pyrimidin-7(6H)-one

Prepared from Description 18 according to the procedure of Example 3 (Method 2). ¹H NMR (500 MHz, DMSO): δ 9.18 (1H, s), 7.67 (2H, d, J8.6), 7.51 (2H, d, J8.6), 2.44 (2H, t, J7.4), 2.32-2.22 (2H, m), 1.89-1.83 (2H, m). m/z (ES⁺) 374 (M+H⁺).

### EXAMPLE 10

### 1-(4-Chlorophenyl)-9-methyl-2-(5,5,5-trifluoropentyl)-1,9-dihydro-6H-purin-6-one

Prepared from Description 16 and Description 22 according to the procedure of Example 3 (Method 2). ¹H NMR (400 MHz, CDCl₃) δ 7.73 (1H, s), 7.53 (2H, d, J4.3), 7.16 (2H, d, J5.7), 3.81 (3H, s), 2.42 (2H, t, J7.4), 2.12-2.00 (2H, m), 1.83-1.75 (2H, m), 1.60-1.51 (2H, m). m/z (ES⁺) 385, 387 (M+H⁺)

### EXAMPLE 11

### 1-(4-Chlorophenyl)-9-methyl-2-[3,4,4,4-tetrafluoro-3-(trifluoromethyl)butyl]-1,9-dihydro-6H-purin-6-one

Description 25 (308 mg, 0.65 mmol) was suspended in phosphorus oxychloride (5 ml, 58.5 mmol) and the reaction heated at 120° C. for 4 h, after which time tlc indicated complete reaction. The reaction was cooled to room temperature, the phosphorus oxychloride evaporated and the mixture was partitioned between ethyl acetate (10 ml) and sat. aqueous potassium carbonate solution (30 ml). The layers were separated, the aqueous phase extracted with more ethyl acetate (20 ml) and the combined organic phases were dried MgSO₄) and evaporated The residue was purified by flash column chromatography on silica (eluent: 5% methanol in dichloromethane with 0.1% NH₃) to give the title compound (230 mg, 78%). ¹H NMR (500 MHz, DMSO) δ 8.09 (1H, s), 7.66 (2H, d, J8.6), 7.47 (2H, d, J8.6), 3.78 (3H, s), 2.78-2.68 (2H, m), 2.62-2.58 (2H, m); m/z (ES⁺) 457,459 (M+H⁺).

### EXAMPLE 12

### 1-(4-Chlorophenyl)-9-methyl-2-(3-phenoxypropyl)-1,9-dihydro-6H-purin-6-one

Prepared from Description 4,4-phenoxybutyric acid and 4-chloroaniline according to the methods of Descriptions 7, 24, 14 and Example 11 respectively. ¹H NMR (360 MHz, DMSO) δ 8.05 (1H, s), 7.62 (2H, d, J8.5), 7.42 (2H, d, J8.5), 7.24 (2H, t, J7.5), 6.90 (1H, t, J7.3), 6.81 (2H, d, J7.9), 3.95 (2H, d, J6.2), 3.74 (3H, s), 2.56-2.49 (2H, m), 2.12-2.02 (2H, m); m/z (ES⁺) 395, 397 (M+H⁺).

Examples 13 to 30 were prepared using analogous methods to those described above using the appropriate amino ester, carboxylic acid and aniline in a 4 step sequence similar to that described for Example 12.

| Ex | Compound Name | Data |
|---|---|---|
| 13 | 1-(4-Chlorophenyl)-9-methyl-2-[2- | ¹H NMR (500 MHz, DMSO) 8.42 (1H, s), |
| | (trifluoromethyl)-1,3-thiazol-4-yl]- | 8.22 (1H, s), 7.42 (2H, d, J 8.6), 7.29 (2H, |
| | 1,9-dihydro-6H-purin-6-one | d, J 8.6), 3.82 (3H, s); m/z (ES⁺) 412, |
| | | 414 (M + H⁺). |
| 14 | 1-(4-Chlorophenyl)-9-ethyl-2-(4,4,4- | ¹H NMR (400 MHz, DMSO) δ 8.13 (1H, |
| | trifluorobutyl)-1,9-dihydro-6H-purin- | s), 7.63 (2H, d, J 8.6), 7.43 (2H, d, J 8.6), |
| | 6-one | 4.20 (2H, q, J 7.2), 2.42 (2H, t, J 7.1), |
| | | 2.38-2.22 (2H, m), 1.89 (2H, q, J 7.6), |
| | | 1.44 (3H, t, J 7.2); m/z (ES⁺) 385, 387 (M + H⁺). |
| 15 | 1-(4-Chlorophenyl)-9-cyclopropyl-2- | ¹H NMR (500 MHz, DMSO) δ 8.09 (1H, |
| | (4,4,4-trifluorobutyl)-1,9-dihydro-6H- | s), 7.63 (2H, d, J 8.6), 7.43 (2H, d, J 8.6), |
| | purin-6-one | 3.56-3.54 (1H, m), 2.43 (2H, t, J 7.1), |
| | | 2.34-2.29 (2H, m), 1.94-1.87 (2H, m), |
| | | 1.13-1.07 (4H, m); m/z (ES⁺) 397, |
| | | 399 (M + H⁺). |
| 16 | 9-Ethyl-1-(4-fluorophenyl)-2-(4,4,4- | ¹H NMR (400 MHz, DMSO) δ 8.13 (1H, |
| | trifluorobutyl)-1,9-dihydro-6H-purin- | s), 7.45-7.38 (4H, m), 4.20 (2H, q, J 7.2), |
| | 6-one | 2.41 (2H, t, J 7.2), 2.36-2.25 (2H, m), |
| | | 1.89 (2H, q, J 8.0), 1.44 (3H, t, J 7.2); m/z (ES⁺) |
| | | 369 (M + H⁺). |
| 17 | 1-(3,4-Difluorophenyl)-9-ethyl-2- | ¹H NMR (400 MHz, DMSO) δ 8.14 (1H, |
| | (4,4,4-trifluorobutyl)-1,9-dihydro-6H- | s), 7.72-7.62 (2H, m), 7.34-7.28 (1H, m), |
| | purin-6-one | 4.20 (2H, q, J 7.3), 2.50 (2H, t, J 7.2), |
| | | 2.35-2.24 (2H, m), 1.94-1.87 (2H, m), |
| | | 1.44 (3H, t, J 7.3); m/z (ES⁺) 387 (M + H⁺). |
| 18 | 1-(4-Chloro-3-fluorophenyl)-9-ethyl- | ¹H NMR (400 MHz, DMSO) δ 8.15 (1H, |
| | 2-(4,4,4-trifluorobutyl)-1,9-dihydro- | s), 7.81 (1H, t, J 8.4), 7.66 (1H, dd, J 2.2, |
| | 6H-purin-6-one | 9.9), 7.34 (1H, d, J 8.5), 4.21 (2H, q, J |
| | | 7.2), 2.49-2.43 (2H, m), 2.37-2.22 (2H, m), |
| | | 1.94-1.86 (2H, m), 1.44 (3H, t, J 7.2); m/z |
| | | (ES⁺) 403, 405 (M + H⁺). |
| 19 | 1-(4-Chlorophenyl)-2-(4,4,4- | ¹H NMR (400 MHz, DMSO) δ 8.20 (1H, |
| | trifluorobutyl)-9-(2,2,2- | s), 7.65 (2H, d, J 8.6), 7.48 (2H, d, J 8.6), |
| | trifluoroethyl)-1,9-dihydro-6H-purin- | 5.18 (2H, q, J 9.2), 2.43 (2H, t, J 6.9), |
| | 6-one | 2.35-2.23 (2H, m), 1.92-1.86 (2H, m).; m/z |
| | | (ES⁺) 439 (M + H⁺). |
| 20 | 1-(4-Fluorophenyl)-9-propyl-2-(4,4,4- | ¹H NMR (400 MHz, DMSO) δ 8.11 (1H, |
| | trifluorobutyl)-1,9-dihydro-6H-purin- | s), 7.46-7.38 (4H, m), 4.13 (2H, t, J 6.9), |
| | 6-one | 2.41 (2H, t, J 7.0), 2.35-2.23 (2H, m), |
| | | 1.91-1.81 (4H, m), 0.88 (3H, t, J 7.4); m/z |
| | | (ES⁺) 383 (M + H⁺). |
| 21 | 1-(4-Chlorophenyl)-9-propyl-2- | ¹H NMR (360 MHz, DMSO) δ 8.12 (1H, |
| | (4,4,4-trifluorobutyl)-1,9-dihydro-6H- | s), 7.63 (2H, d, J 8.6), 7.44 (2H, d, J 8.6), |
| | purin-6-one | 4.14 (2H, t, J 6.9), 2.44-2.36 (2H, m), |
| | | 2.33-2.23 (2H, m), 1.92-1.82 (4H, m), |
| | | 0.88 (3H, t, J 7.4); m/z (ES⁺) 399 (M + H⁺). |
| 22 | 9-Ethyl-1-(3-fluoro-4-methylphenyl)- | ¹H NMR (360 MHz, DMSO) δ 8.13 (1H, |
| | 2-(4,4,4-trifluorobutyl)-1,9-dihydro- | s), 7.47 (1H, t, J 8.3), 7.31 (1H, d, J 10.2), |
| | 6H-purin-6-one | 7.13 (1H, d, J 10.0), 4.20 (2H, q, J 7.3), |
| | | 2.45 (2H, t, J 7.2), 2.37-2.21 (5H, m), |
| | | 1.93-1.85 (2H, m), 1.44 (3H, t, J 7.3); m/z |
| | | (ES⁺) 383 (M + H⁺). |
| 23 | 1-(4-Chlorophenyl)-9-methyl-2-(4- | ¹H NMR (360 MHz, DMSO) δ 8.05 (1H, s), |
| | methylpentyl)-1,9-dihydro-6H-purin- | 7.63 (2H, d, J 8.6), 7.43 (2H, d, J 8.6), |
| | 6-one | 3.75 (3H, s), 2.36-2.28 (2H, m), 1.63-1.52 (2H, |
| | | m), 1.58-1.42 (1H, m), 1.15-1.02 (2H, m), |
| | | 0.79 (6H, d, J 6.6); m/z (ES⁺) 345, |
| | | 347 (M + H⁺). |
| 24 | 1-(4-Chlorophenyl)-2-cyclohexyl-9- | ¹H NMR (400 MHz; CDCl₃) 7.71 (1H, s), |
| | methyl-1,9-dihydro-6H-purin-6-one | 7.51 (2H, d, J 8), 7.16 (2H, d, J 8), 3.81 (3H, |
| | | s), 2.35-2.2 (1H, m), 1.8-1.6 (7H, m), |
| | | 1.3-1.15 (1H, m), 1.1-0.95 (2H, m); m/z (ES⁺) |
| | | 343, 345 (M + H⁺). |
| 25 | 1-(4-Chlorophenyl)-9-methyl-2-(3- | ¹H NMR (400 MHz; CDCl₃) 7.71 (1H, s), |
| | methylbutyl)-1,9-dihydro-6H-purin-6- | 7.51 (2H, d, J 7), 7.17 (2H, d, J 7), 3.82 (3H, |
| | one | s), 2.45-2.35 (2H, m), 1.6-1.4 (3H, m), |
| | | 0.78 (6H, d, J 6.5); m/z (ES⁺) 331, 333 (M + H⁺). |
| 26 | 1-(4-Chlorophenyl)-9-ethyl-2-(3- | ¹H NMR (400 MHz; CDCl₃) 7.75 (1H, s), |
| | methylbutyl)-1,9-dihydro-6H-purin-6- | 7.51 (2H, d, J 7), 7.17 (2H, d, J 7), 4.23 (2H, |
| | one | q, J 7), 2.45-2.35 (2H, m), 1.55 (3H, t, J 7), |
| | | 1.6-1.4 (3H, m), 0.78 (6H, d, J 6.5); m/z |
| | | (ES⁺) 345, 347 (M + H⁺). |
| 27 | 9-Ethyl-1-(4-fluorophenyl)-2-(3- | ¹H NMR (360 MHz; CDCl₃) 7.76 (1H, s), |
| | methylbutyl)-1,9-dihydro-6H-purin-6- | 7.3-7.15 (4H, m), 4.23 (2H, q, J 7), |
| | one | 2.45-2.35 (2H, m), 1.6-1.4 (6H, m), 0.78 (6H, d, |
| | | J 6.5); m/z (ES⁺) 329 (M + H⁺). |
| 28 | 2-tert-Butyl-1-(4-chlorophenyl)-9- | ¹H NMR (400 MHz; CDCl₃) 7.75 (1H, s), |
| | methyl-1,9-dihydro-6H-purin-6-one | 7.46 (2H, d, J 7), 7.18 (2H, d, J 7), |
| | | 3.81 (3H, s), 1.20 (9H, s); m/z (ES⁺) 317, |
| | | 319 (M + H⁺). |
| 29 | 1-(4-Chlorophenyl)-9-methyl-2-{2- | ¹H NMR (500 MHz, CDCl₃) 2.75 (2H, t, J |
| | [5-(trifluoromethyl)pyridin-3- | 7.2); 3.16 (2H, t, J 7.2); 3.82 (3H, s); |
| | yl]ethyl}-1,9-dihydro-6H-purin-6-one | 7.11 (2H, d, J 8.6); 7.52 (2H, d, J 8.6); 7.72 (1H, |
| | | s); 7.73 (1H, s); 8.60 (1H, s); 8.72 (1H, |
| | | s); m/z (ES⁺) 434 (M + H⁺). |
| 30 | 1-(4-Chlorophenyl)-9-methyl-2-[2- | ¹H NMR (500 MHz, CDCl₃) 7.75 (1H, s), |
| | (trifluoromethyl)-4,5,6,7-tetrahydro- | 7.52 (2H, d, J 8.4), 7.22-7.15 (2H, m), |
| | 1,3-benzothiazol-5-yl]-1,9-dihydro- | 3.80 (3H, s), 3.4-3.3 (1H, m), 3.1-3.0 (1H, m), |
| | 6H-purin-6-one | 3.0-2.9 (1H, m), 2.9-2.8 (1H, m), |
| | | 2.65-2.55 (1H, m), 2.25-2.10 (2H, m); m/z (ES⁺) 466, |
| | | 468 (M + H⁺). |

### EXAMPLE 31

### 1-(4-Chlorophenyl)-2-(2-cyclopentylethyl)-9-methyl-1,9-dihydro-6H-purin-6-one

3-Cyclopentylpropanoyl chloride (26 μL, 0.17 mmol) was added to a solution of Description 16 (35 mg, 0.14 mmol) and ethyldiisopropylamine (60 μL, 0.35 mmol) in THF (1 ml) at room temperature. The mixture was stirred for 60 h, then partitioned between ethyl acetate (25 ml) and water (25 ml). The layers were separated and the organic layer was dried (Na₂SO₄) and evaporated. To the residue (59 mg), phosphorus oxychloride (1 ml) was added and the mixture heated at 100° C. for 4 h. The mixture was cooled to room temperature, partitioned between ethyl acetate (25 ml) and 10% aqueous potassium carbonate solution (25 ml) and the layers separated. The aqueous phase was extracted with more ethyl acetate (10 ml) and the combined organic layers were dried (Na₂SO₄) and evaporated. Purification by preparative tlc (eluant: 5% MeOH in CH₂Cl₂) afforded the title compound (10 mg). ¹H NMR (400 M; CDCl₃) 7.73 (1H, s), 7.51 (2H, d, J7), 7.17 (2H, d, J7), 3.82 (3H, s), 2.45-2.35 (2H, m), 1.70-1.40 (9H, m), 1.0-0.85 (2H, m); m/z (ES⁺) 357, 359 (M+H⁺).

### EXAMPLE 32

### 1-(4-Chlorophenyl)-2-(2-cyclopropylethyl)-9-ethyl-1,9-dihydro-6H-purin-6-one

Prepared from Description 39 according to the procedure of Example 11. ¹H NMR (500 MHz, DMSO) δ 8.16 (1H, s), 7.64 (2H, d, J8.6), 7.44 (2H, d, J8.6), 4.19 (2H, q, J7.3), 2.43 (2H, t, J7.5), 1.52 (2H, q, J 7.2), 1.44 (3H, t, J7.3), 0.69-0.59 (1H, m), 0.37-0.25 (2H, m), ⁻0.07-⁻0.15 (2H, m); m/z (ES⁺) 343, 345 (M+H⁺).

### EXAMPLE 33

### 1-(4-chlorophenyl)-9-methyl-2-[(4-methyl-1,2,5-oxadiazol-3-yl)methyl]-1,9-dihydro-6H-purin-6-one

Description 40 (46 mg, 0.12 mmol) and polyphosphoric acid (0.24 ml) were combined and heated at 150° C. for 15 minutes after which time TLC indicated the reaction was complete. The reaction mixture was partitioned between dichloromethane (20 ml) and 10% aqueous potassium carbonate solution (30 ml), the layers separated and the aqueous phase extracted with dichloromethane (20 ml). The combined organic layers were dried over MgSO₄, evaporated and the residue purified by mass directed preparative HPLC to give the title compound (11 mg, 25%). ¹H NMR (500 MHz, DMSO) δ 8.08 (1H, s), 7.67 (2H, d, J 8.3), 7.51 (2H, d, J8.3), 4.00 (2H, s), 3.61 (3H, s), 2.53 (3H, s); m/z (ES⁺) 357, 359 (M+H⁺).

### EXAMPLE 34

### 1-(4-Chlorophenyl)-9-methyl-2-(1-methyl-2-[trifluoromethyl)-1,3-thiazol-4-yl]ethyl)-1,9-dihydro-6H-purin-6-one

Prepared from Description 16 and Description 44, according to the methods of Description 39 and Example 33 respectively. ¹H NMR (400 MHz, DMSO) δ 8.06 (1H, s), 7.67 (1H, s), 7.64 (1H, dd, J2.4, 8.4), 7.53-7.47 (2H, m), 6.92 (1H, dd, J2.5, 8.4), 3.77 (3H, s), 3.42-3.35 (1H, m), 2.99-2.89 (2H, m), 1.16 (3H, d, J6.4); m/z (ES⁺) 454, 456 (M+H⁺).

### EXAMPLE 35

### 1-(4-Chlorophenyl)-9-methyl-2-(4,4,4-trifluoro-1-methylbutyl)-1,9-dihydro-6H-purin-6-one

Prepared from Description 4, Description 45 and 4-chloroaniline according to Descriptions 7, 24, 25, and Example 33 respectively. ¹H NMR (360 M CDCl₃) δ 7.74 (1H, s), 7.53 (2H, dd, J2.8, 5.9), 7.20-7.13 (1H, t, m), 7.10-7.07 (1H, m), 3.82 (3H, s), 2.64-2.58 (1H, m), 2.22-2.14 (1H, m), 2.04-1.96 (2H, m), 1.78-1.70 (1H, m), 1.19 (3H, d, J6.7); m/z (ES⁺) 385, 387 (M+H⁺).

### EXAMPLE 36

### 1-(5-Chloro-1,3-thiazol-2-yl)-9-methyl-2-(2,4,6-trifluorobenzyl)-1,9-dihydro-6H-purin-6-one

A mixture of N-(5-Chloro-1,3-thiazol-2-yl)-1-methyl-5-{[(2,4,6-trifluorophenyl)acetyl]amino}-1H-imidazole-4-carboxamide (250 mg, 0.58 mmol) and phosphorus oxychloride (1.5 mL) was heated to 110° C. for 1 h. After cooling to room temperature, the excess POCl₃ was removed in vacuo, and ice water (10 mL) was added to the residue. The mixture was extracted with CH₂Cl₂ (20 mL), and the CH₂Cl₂ extract was washed with saturated NaHCO₃ (10 mL) and brine (10 mL), dried (Na₂SO₄), filtered, and evaporated to give a brown oily residue. Purification by preparative plate chromatography (5% MeOH/CH₂Cl₂) provided the title compound as a yellow solid (40 mg, 17%). ¹H NMR (400 MHz, CDCl₃) 7.71 (1H, s), 7.67 (1H, s), 6.67 (2H, t, J8), 3.99 (2H, s), 3.64 (3H, s); m/z (ES⁺) 411.91 (M+H⁺).

### EXAMPLE 37

### 1-(4-Chlorophenyl)-9-methyl-2-(2,4,6-trifluorobenzyl)-1,9-dihydro-6H-purin-6-one

Description 16 (50 mg, 0.20 mmol) was combined with 2,4,6-trifluorophenylacetic acid (76 mg, 0.40 mmol) and polyphosphoric acid (2.0 g) in a sealed tube. The mixture was heated to 140° C. and stirred for 30 mins. On cooling, ice was added and the polyphosphoric acid plug broken up and the suspension was basified to pH 9.0 using 4N aqueous NaOH. The aqueous phase was extracted with CH₂Cl₂ (3×20 mL) and the combined organic fractions dried over MgSO₄, filtered and concentrated in vacuo. The resulting yellow oil was triturated with Et₂O to afford the title compound as a white solid (45 mg, 56% yield). ¹H NMR (400 MHz, DMSO-D6) 8.04 (1H, s), 7.61 (2H, d), 7.44 (2H, d), 7.11 (2H, t), 3.73 (2H, s), 3.56 (3H, s); m/z (ES⁺) 404.98 (M+H⁺).

### EXAMPLE 38

### 1-(4-chlorophenyl)-9-ethyl-2-((6-morpholinopyridin-3-yl)methyl)-1H-purin-6(9H)-one

1-(4-chlorophenyl)-2-((6-chloropyridin-3-yl)methyl)-9-ethyl-1H-purin-6(9H)-one [prepared according to the procedures in Description 14 and Example 3] (40 mg, 0.1 mmol) was mixed with morpholine (2 mL) and CsF (61 mg, 0.4 mmol) and the mixture was heated at 100° C. for 16 h. The reaction was cooled to room temperature, concentrated, and partitioned between ethyl acetate (35 mL) and water (25 mL). The layers were separated, the aqueous phase extracted with more ethyl acetate (25 mL) and the combined organic phases were dried (MgSO₄) and evaporated. The residue was purified by preparative TLC to give the title compound.

### EXAMPLE 39

### 1-(4-chlorophenyl)-9-ethyl-2-((6-ethylpyridin-3-yl)methyl)-1H-purin-6(9H)one

Triethylaluminum (2M in THF, 0.2 mL, 0.4 mmol) and Pd(PPh₃)₄ (6 mg, 0.005 mmol) were added to the solution of 1-(4-chlorophenyl)-2-((6-chloropyridin-3-yl)methyl)-9-ethyl-1H-purin-6(9H)-one [prepared according to the procedures in Description 14 and Example 3] (40 mg, 0.1 mmol) in THF (10 mL) and the mixture was heated at 80° C. for 16 h. The reaction was cooled to room temperature, diluted with CH₂Cl₂ (30 mL), and saturated aqueous sodium potassium tartrate (10 mL) was added, followed by saturated aqueous ammonium chloride solution (10 mL). The mixture was stirred vigorously for 1 h and then allowed to settle for 1 h before separation of the phases. The aqueous phase was extracted with dichloromethane (50 mL) and the combined organic extracts washed with 1M sodium potassium tartrate, dried over MgSO₄, filtered and concentrated. The residue was purified by preparative TLC to give the title compound.

### EXAMPLE 40

### 3-((1-(4-chlorophenyl)-9-ethyl-6-oxo-6,9-dihydro-1H-purin-2-yl)methyl)benzonitrile

Zn(CN)₂ (10 mg, 0.084 mmol), DPPF (3 mg, 0.005 mmol), and Pd₂(dba)₃ (5 mg, 0.005 mmol) were added to the solution of 2-(3-bromobenzyl)-1-(4-chlorophenyl)-9-ethyl-1H-purin-6(9H)-one [prepared according to the procedures in Description 14 and Example 3] (60 mg, 0.14 mmol) in DMF-H₂O (10:1, 3 mL) and the mixture was heated at 120° C. for 6 h. The reaction was cooled to room temperature, partitioned between ethyl acetate (50 mL) and water (30 mL). The layers were separated, the aqueous phase extracted with more ethyl acetate (40 mL) and the combined organic phases were dried (MgSO₄) and evaporated. The residue was purified by preparative TLC to give the title compound.

### EXAMPLE 41

### 1-(4-chlorophenyl)-9-ethyl-2-(3-fluorobenzoyl)-1H-purin-6(9H)-one

SeO₂ (16 mg, 0.14 mmol) was added to the solution of 2-(3-fluorobenzyl)-1-(4-chlorophenyl)-9-ethyl-1H-purin-6(9H)-one [prepared according to the procedures in Description 14 and Example 3] (50 mg, 0.13 mmol) in dioxane (5 mL) and the mixture was heated at 110° C. for 16 h. The reaction was cooled to room temperature, concentrated, and partitioned between ethyl acetate (50 mL) and water (30 mL). The layers were separated, the aqueous phase extracted with more ethyl acetate (40 mL) and the combined organic phases were dried (MgSO₄) and evaporated. The residue was purified by preparative TLC to give the title compound.

### EXAMPLE 42

### 1-(4-chlorophenyl)-9-ethyl-2-(1-(3-(trifluoromethyl)phenyl)ethyl)-1H-purin-6(9H)-one

NaHMDS (1M in THF, 074 mL, 0.74 mmol) was added dropwise to a cooled solution (−78° C.) of 2-(3-(trifluoromethyl)benzyl)-1-(4-chlorophenyl)-9-ethyl-1H-purin-6(9H)-one [prepared according to the procedures in Description 14 and Example 3] (160 mg, 0.37 mmol) in THF (15 mL) and the mixture was stirred at −78° C. for 1 h. Iodomethane (35 μl, 0.56 nmol) was added and the mixture was stirred at −78° C. for 1 h followed 1 h at 0° C. The reaction was quenched with sat NaHCO₃ (20 mL) and extracted with ethyl acetate (3×30 mL) and the combined organic phases were dried (MgSO₄) and evaporated. The residue was purified by preparative TLC to give the title compound.

### EXAMPLE 43

### 1-(4-Chlorophenyl)-9-ethyl-2-[3-(trifluoromethyl)benzyl]-1,9-dihydro-6H-purin-6-one

Prepared from Description 76 according to the procedure of Example 3 (Method 1). m/z (ES⁺) 433 (M+H⁺), 435.

### EXAMPLE 44

### 1-(4-Chlorophenyl)-9-cyclopropyl-2-(2,4,6-trifluorobenzyl)-1,9-dihydro-6H-purin-6-one

Prepared from Description 69 and 2,4,6-trifluorophenylacetic acid according to the procedure of Example 3 (Method 2). m/z (ES⁺) 431 (M+H⁺).

### EXAMPLE 45

### 1-(4-Chlorophenyl)-9-cyclopropyl-2-(2,3-difluorobenzyl)-1,9-dihydro-6H-purin-6-one

Prepared from Description 69 and 2,3-trifluorophenylacetic acid according to the procedure of Example 3 (Method 2). m/z (ES⁺) 413 (M+H⁺), 415.

### EXAMPLE 46

### 1-(4-Chlorophenyl)-9-cyclopropyl-2-(2,4-difluorobenzyl)-1,9-dihydro-6H-purin-6-one

Prepared from Description 69 and 2,4-trifluorophenylacetic acid according to the procedure of Example 3 (Method 2). m/z (ES⁺) 413 (M+H⁺).

### EXAMPLE 47

### 1-(4-Chlorophenyl)-9-cyclopropyl-2-(3,5-difluorobenzyl)-1,9-dihydro-6H-purin-6-one

Prepared from Description 69 and 3,5-trifluorophenylacetic acid according to the procedure of Example 3 (Method 2). m/z (ES⁺) 413 (M+H⁺).

### EXAMPLE 48

### 1-(4-Chlorophenyl)-9-cyclopropyl-2-(5,5,5-trifluoropentyl)-1,9-dihydro-6H-purin-6-one

Prepared from Description 69 and 6,6,6-trifluoro-hexanoic acid according to the procedure of Example 3 (Method 2). m/z (ES⁺) 411 (M+H⁺), 413.

### EXAMPLE 49

### 1-(4-Chlorophenyl)-9-cyclopropyl-2-(1,2,3,4-tetrahydronaphthalen-2-yl)-1,9-dihydro-6H-purin-6-one

Prepared from Description 69 and 1,2,3,4-tetrahydro-naphthalene-2-carboxylic acid according to the procedure of Example 3 (Method 2). m/z (ES⁺) 417 (M+H⁺).

### EXAMPLE 50

### 6-(4-Chlorophenyl)-5-(2,4,6-trifluorobenzyl)[1,3]thiazolo[5,4-d]pyrimidin-7(6H)-one

Prepared from Description 17 and 2,4,6-trifluorophenylacetic acid according to the procedure of Example 3 (Method 2). m/z (ES⁺) 408 (M+H⁺), 410.

### EXAMPLE 51

### 6-(4-Chlorophenyl)-5-(2,3-difluorobenzyl)-[1,3]thiazolo[5,4-d]pyrimidin-7(6H)-one

Prepared from Description 17 and 2,4-trifluorophenylacetic acid according to the procedure of Example 3 (Method 2). m/z (ES⁺) 390 (M+H⁺).

### EXAMPLE 52

### 6-(4-Chlorophenyl)-5-(3,5-difluorobenzyl)[1,3]thiazolo[5,4-d]pyrimidin-7(6H)-one

Prepared from Description 17 and 3,5-trifluorophenylacetic acid according to the procedure of Example 3 (Method 2). m/z (ES⁺) 390, (M+H⁺), 392.

### EXAMPLE 53

### 6-(4-Chlorophenyl)-5-(2,4-difluorobenzyl)[1,3]thiazolo[5,4-d]pyrimidin-7(6H)-one

Prepared from Description 17 and 2,4-trifluorophenylacetic acid according to the procedure of Example 3 (Method 2). m/z (ES⁺) 390 (M+H⁺).

### EXAMPLE 54

### 6-(4-Chlorophenyl)-5-(5,5,5-trifluoropentyl)-[1,3]thiazolo[5,4-d]pyrimidin-7(6H)-one

Prepared from Description 17 and 6,6,6-trifluoro-hexanoic acid according to the procedure of Example 3 (Method 2). m/z (ES⁺) 388 (M+H⁺), 390.

### EXAMPLE 55

### 6-(4-Chlorophenyl)-5-(4,4,4-trifluoro-3-methylbutyl)[1,3]thiazolo[5,4-d]pyrimidin-7(6H)-one

Prepared from Description 17 and 5,5,5-trifluoro-4-methyl-pentanoic acid according to the procedure of Example 3 (Method 2). m/z (ES⁺) 388 (M+H⁺).

### EXAMPLE 56

### 1-(4-Chlorophenyl)-9-ethyl-2-(5,5,5-trifluoropentyl)-1,9-dihydro-6H-purin-6-one

Prepared from Description 1 and 6,6,6-trifluoro-hexanoic acid according to the procedure of Example 3 (Method 2). m/z (ES⁺) 399 (M+H⁺), 401.

### EXAMPLE 57

### 1-(4-Chlorophenyl)-9-ethyl-2-(2-ethyl-1,3-thiazol-4-yl)methyl]-1,9-dihydro-6H-purin-6-one

Prepared from Description 1 and Description 73 according to the procedure of Example 3 (Method 2). m/z (ES⁺) 400 (M+H⁺), 402.

### EXAMPLE 58

### 6-(4-Chlorophenyl)-5-(4,4,4-trifluoro-2-methylbutyl)[1,3]thiazolo[5,4-d]pyrimidin-7(6H)-one

Prepared from Description 17 and 5,5,5-trifluoro-3-methylpentanoic acid according to the procedure of Example 3 (Method 2). m/z (ES⁺) 388 (M+H⁺), 385, 386

### EXAMPLE 59

### 6-(4-Chlorophenyl)-5-(1,2,3,4-tetrahydronaphthalen-2-yl)[1,3]thiazolo[5,4-d]pyrimidin-7(6H)-one

Prepared from Description 17 and 1,2,3,4-tetrahydronaphthalene-2-carboxylic acid according to the procedure of Example 3 (Method 2). m/z (ES⁺) 394 (M+H⁺).

### EXAMPLE 60

### 6-(4-Chlorophenyl)-5-cyclohexylmethyl)[1,3]thiazolo[5,4-d]pyrimidin-7-(6H)-one

Prepared from Description 17 and cyclohexylacetic acid according to the procedure of Example 3 (Method 2). m/z (ES⁺) 360 (M+H⁺).

### EXAMPLE 61

### 1-(4-chlorophenyl)-2-(cyclohexylmethyl)-9-cyclopropyl-1H-purin-6(9H)-one

Prepared from Description 69 and cyclohexylacetic acid according to the procedure of Example 3 (Method 2). m/z (ES⁺) 383 (M+H⁺).

### EXAMPLE 62

### 1-(4-chlorophenyl)-9-ethyl-2-[(5-methyl-2-phenyl-1,3-oxazol-4-yl)methyl]-1,9-dihydro-6H-purin-6-one

Prepared from Description 1,2-(5-methyl-2-phenyloxazol-4-yl)acetic acid and 4-chloroaniline according to the methods of Descriptions 7, 24, 14 and Example 11 respectively.

### EXAMPLE 63

### 1-(4-chlorophenyl)-9-ethyl-2-[(5-methyl-2-phenyl-1,3-thiazol-4-yl)methyl]-1,9-dihydro-6H-purin-6-one

Prepared from Description 1,2-(5-methyl-2-phenylthiazol-4-yl)acetic acid and 4-chloroaniline according to the methods of Descriptions 7, 24, 14 and Example 11 respectively.

### EXAMPLE 64

### 1-(4-chlorophenyl)-9-ethyl-2-[(2-phenyl-1,3-thiazol-4-yl)methyl]-1,9-dihydro-6H-purin-6-one

Prepared from Description 1, 2-(2-phenylthiazol-4-yl)acetic acid and 4-chloroaniline according to the methods of Descriptions 7, 24, 14 and Example 11 respectively.

### EXAMPLE 65

### 1-(4-chlorophenyl)-2-(2-chloropyridin-4-yl)-9-ethyl 1,9-dihydro-6H-purin-6-one

Prepared from Description 1,2-chloroisonicotinic acid and 4-chloroaniline according to the methods of Descriptions 7, 24, 14 and Example 11 respectively.

### EXAMPLE 66

### 1-(4-chlorophenyl)-9-ethyl-2-[fluoro(3-fluorophenyl)methyl]-1,9-dihydro-6H-purin-6-one

Prepared in two steps from Description 38 and 2-fluoro-2-(3-fluorophenyl)acetic acid using the procedures given in description 39 and example 3 (method 1), respectively. m/z (ES⁺) 400 (M+H⁺).

### EXAMPLE 67

### 1-(4-chlorophenyl)-2-[(2,6-difluorophenyl)(fluoro)methyl]-9-ethyl-1,9-dihydro-6H-purin-6-one

Prepared in two steps from Description 38 and 2-(3,5-difluorophenyl)-2-fluoroacetic acid using the procedures given in description 39 and example 3 (method 1), respectively. m/z (ES⁺) 418 (M+H⁺).

### EXAMPLE 68

### 1-(4-chlorophenyl)-2-[(3,4-difluorophenyl)(fluoro)methyl]-9-ethyl-1,9-dihydro-6H-purin-6-one

Prepared in two steps from Description 38 and 2-(3,4-difluorophenyl)-2-fluoroacetic acid using the procedures given in description 39 and example 3 (method 1), respectively. m/z (ES⁺) 418 (M+H⁺).

### EXAMPLE 69

### 1-(4-chlorophenyl)-2-[(3,4-difluorophenyl)(difluoro)methyl]-9-ethyl-1,9-dihydro-6H-purin-6-one

Prepared in two steps from Description 38 and 2-(3,4-difluorophenyl)-2,2-difluoroacetic acid using the procedures given in description 39 and example 3 (method 1), respectively. m/z (ES⁺) 436 (M+H⁺).

### EXAMPLE 70

### 1-(4-chlorophenyl)-2-[(4,4-difluorocyclohexyl)methyl]-9-ethyl-1,9-dihydro-6H-purin-6-one

Prepared in two steps from Description 38 and Description 62 using the procedures given in description 39 and example 3 (method 1), respectively. m/z (ES⁺) 407 (M+H⁺).

### EXAMPLE 71

### 1-(4-chlorophenyl)-2-[(4,4-difluorocyclohexyl)methyl]-9-methyl-1,9-dihydro-6H-purin-6-one

Prepared in two steps from Description 17 and Description 62 using the procedures given in description 39 and example 3 (method 1), respectively. m/z (ES⁺) 393 (M+H⁺).

### EXAMPLE 72

### 1-(4-chlorophenyl)-9-ethyl-2-[1-(5-methyl-2-phenyl-1,3-oxazol-4-yl)ethyl]-1,9-dihydro-6H-purin-6-one

Prepared from 1-(4-chlorophenyl)-9-ethyl-2-((5-methyl-2-phenyloxazol-4-yl)methyl)-1H-purin-6(9H)-one using the procedure given in example 42. m/z (ES⁺) 460 (M+H⁺).

### EXAMPLE 73

### 1-(4-chlorophenyl)-2-[1-(3,5-difluorophenyl)ethyl]-9-ethyl-1,9-dihydro-6H-purin-6-one

Prepared from 2-(3,5-difluorobenzyl)-1-(4-chlorophenyl)-9-ethyl-1H-purin-6(9H)-one using the procedure given in example 42. m/z (ES⁺) 414 (M+H⁺).

### EXAMPLE 74

### 1-(4-chlorophenyl)-2-[1-(2,6-difluorophenyl)ethyl]-9-ethyl-1,9-dihydro-6H-purin-6-one

Prepared from 2-(2,6-difluorobenzyl)-1-(4-chlorophenyl)-9-ethyl-1H-purin-6(9H)-one using the procedure given in example 42. m/z (ES⁺) 414 (M+H⁺).

### EXAMPLE 75

### 1-(4-chlorophenyl)-2-[2-(2,3-dihydro-1-benzofuran-2-yl)ethyl]-9-ethyl-1,9-dihydro-6H-purin-6-one

Prepared from description 38 and 3-(2,3-dihydrobenzofuran-2-yl)propanoic acid (prepared according to procedure found in Organic & Biomolecular Chemistry, 2003, 11, 1930-1937) using procedures analogous to those used in description 39 and example 3 (method 1). m/z (ES⁺) 421.1 (M+H⁺).

### EXAMPLE 76

### 2-[(E)-2-(1-benzofuran-2-yl)vinyl]-1-(4-chlorophenyl)-9-methyl-1,9-dihydro-6H-purin-6-one

Prepared from description 16 and 3-(2,3-dihydrobenzofuran-2-yl)propanoic acid (prepared according to procedure found in Organic & Biomolecular Chemistry, 2003, 11, 1930-1937) using procedures analogous to those used in description 39 and example 3 (method 1). m/z (ES⁺) 407.1 (M+H⁺).

### EXAMPLE 77

### 1-(4-chlorophenyl)-2-(2,3-dihydro-1-benzofuran-3-ylmethyl)-9-ethyl-1,9-dihydro-6H-purin-6-one

Prepared from description 38 and 2-(2,3-dihydrobenzofuran-3-yl)acetic acid (prepared as described in WO 2001/14358) using procedures analogous to those used in description 38 and example 3 (method 1). m/z (ES⁺) 407.1078 (M+H⁺).

### EXAMPLE 78

### 1-(4-chlorophenyl)-2-(2,3-dihydro-1-benzofuran-3-ylmethyl)-9-methyl-1,9-dihydro-6H-purin-6-one

Prepared from description 16 and 2-(2,3-dihydrobenzofuran-3-yl)acetic acid (prepared as described in WO 2001/14358) using procedures analogous to those used in description 38 and example 3 (method 1). m/z (ES⁺) 393.09 (M+H⁺).

Examples 79 to 265 were prepared using methods analogous to those described in Example 37 or those described in Description 39 and Example 3 (method 1) using the appropriate amino amide and carboxylic acid.

| Ex | Compound Name | MS Data |
|---|---|---|
| 79 | 1-(4-chlorophenyl)-2-[(2,5-dimethyl-1,3-thiazol-4- | 400.17 |
| | yl)methyl]-9-ethyl-1,9-dihydro-6H-purin-6-one | |
| 80 | 2-(3-chlorobenzyl)-1-(4-chlorophenyl)-9-ethyl-1,9- | 399.13 |
| | dihydro-6H-purin-6-one | |
| 81 | 1-(4-chlorophenyl)-9-ethyl-2-(3-methylbenzyl)-1,9- | 379.18 |
| | dihydro-6H-purin-6-one | |
| 82 | 1-(4-chlorophenyl)-9-ethyl-2-(3-methoxybenzyl)- | 395.18 |
| | 1,9-dihydro-6H-purin-6-one | |
| 83 | 2-(3-bromobenzyl)-1-(4-chlorophenyl)-9-ethyl-1,9- | 445.10 |
| | dihydro-6H-purin-6-one | |
| 84 | 1-(4-chlorophenyl)-9-ethyl-2-(1-phenylethyl)-1,9- | 379.23 |
| | dihydro-6H-purin-6-one | |
| 85 | 1-(4-chlorophenyl)-9-ethyl-2-(2-fluorobenzyl)-1,9- | 383.20 |
| | dihydro-6H-purin-6-one | |
| 86 | 1-(4-chlorophenyl)-9-ethyl-2-(4-fluorobenzyl)-1,9- | 383.19 |
| | dihydro-6H-purin-6-one | |
| 87 | 1-(4-chlorophenyl)-9-ethyl-2-[1-(3- | 397.23 |
| | fluorophenyl)ethyl]-1,9-dihydro-6H-purin-6-one | |
| 88 | 1-(4-chlorophenyl)-2-(2,3-difluorobenzyl)-9-ethyl- | 401.15 |
| | 1,9-dihydro-6H-purin-6-one | |
| 89 | 1-(4-chlorophenyl)-2-(3,4-difluorobenzyl)-9-ethyl- | 401.15 |
| | 1,9-dihydro-6H-purin-6-one | |
| 90 | 1-(4-chlorophenyl)-2-(3,5-difluorobenzyl)-9-ethyl- | 401.15 |
| | 1,9-dihydro-6H-purin-6-one | |
| 91 | 2-benzyl-1-(4-chlorophenyl)-9-ethyl-1,9-dihydro- | 365.17 |
| | 6H-purin-6-one | |
| 92 | 2-(2-chlorobenzyl)-1-(4-chlorophenyl)-9-ethyl-1,9- | 399.14 |
| | dihydro-6H-purin-6-one | |
| 93 | 1-(4-chlorophenyl)-2-(2,6-difluorobenzyl)-9-ethyl- | 401.16 |
| | 1,9-dihydro-6H-purin-6-one | |
| 94 | 1-(4-chlorophenyl)-2-(2,4-difluorobenzyl)-9-ethyl- | 400.94 |
| | 1,9-dihydro-6H-purin-6-one | |
| 95 | 1-(4-chlorophenyl)-2-(2,5-difluorobenzyl)-9-ethyl- | 400.95 |
| | 1,9-dihydro-6H-purin-6-one | |
| 96 | 1-(4-chlorophenyl)-9-ethyl-2-(3,4,5- | 418.93 |
| | trifluorobenzyl)-1,9-dihydro-6H-purin-6-one | |
| 97 | 1-(4-chlorophenyl)-9-ethyl-2-[2- | 432.91 |
| | (trifluoromethyl)benzyl]-1,9-dihydro-6H-purin-6- | |
| | one | |
| 98 | 1-(4-chlorophenyl)-9-ethyl-2-(2,4,6- | 418.91 |
| | trifluorobenzyl)-1,9-dihydro-6H-purin-6-one | |
| 99 | 1-(4-chlorophenyl)-2-(cyclohexylmethyl)-9-ethyl- | 371.16 |
| | 1,9-dihydro-6H-purin-6-one | |
| 100 | 1-(4-chlorophenyl)-9-ethy-2-(3,3,3-trifluoro-2- | 385.10 |
| | methylpropyl)-1,9-dihydro-6H-purin-6-one | |
| 101 | 1-(4-chlorophenyl)-9-ethyl-2-(1,2,3,4- | 405.06 |
| | tetrahydronaphthalen-2-yl)-1,9-dihydro-6H-purin-6- | |
| | one | |
| 102 | 1-(4-chlorophenyl)-9-ethyl-2-[(4- | 385.09 |
| | methylcyclohexyl)methyl]-1,9-dihydro-6H-purin-6- | |
| | one | |
| 103 | 1-(4-chlorophenyl)-9-ethyl-2-(tetrahydro-2H- | 389.03 |
| | thiopyran-4-ylmethyl)-1,9-dihydro-6H-purin-6-one | |
| 104 | 1-(4-chlorophenyl)-9-methyl-2-(3,3,3-trifluoro-2- | 371.00 |
| | methylpropyl)-1,9-dihydro-6H-purin-6-one | |
| 105 | 1-(4-chlorophenyl)-9-ethyl-2-[(4- | 385.08 |
| | oxocyclohexyl)methyl]-1,9-dihydro-6H-purin-6-one | |
| 106 | 1-(4-chlorophenyl)-9-ethyl-2-(4,4,4-trifluoro-2- | 399.05 |
| | methylbutyl)-1,9-dihydro-6H-purin-6-one | |
| 107 | 1-(4-chlorophenyl)-9-methyl-2-(4,4,4-trifluoro-2- | 385.04 |
| | methylbutyl)-1,9-dihydro-6H-purin-6-one | |
| 108 | 1-(4-chlorophenyl)-9-methyl-2-(1,2,3,4- | 391.11 |
| | tetrahydronaphthalen-2-yl)-1,9-dihydro-6H-purin-6- | |
| | one | |
| 109 | 1-(4-fluorophenyl)-9-methyl-2-(1,2,3,4- | 375.14 |
| | tetrahydronaphthalen-2-yl)-1,9-dihydro-6H-purin-6- | |
| | one | |
| 110 | 9-ethyl-1-(4-fluorophenyl)-2-(4,4,4-trifluoro-2- | 383.12 |
| | methylbutyl)-1,9-dihydro-6H-purin-6-one | |
| 111 | 1-(4-fluorophenyl)-9-methyl-2-(4,4,4-trifluoro-2- | 369.11 |
| | methylbutyl)-1,9-dihydro-6H-purin-6-one | |
| 112 | 1-(4-chlorophenyl)-9-cyclopropyl-2-(4,4,4-trifluoro- | 411.09 |
| | 2-methylbutyl)-1,9-dihydro-6H-purin-6-one | |
| 113 | 1-(4-chlorophenyl)-9-ethyl-2-(4,4,4-trifluoro-3- | 399.01 |
| | methylbutyl)-1,9-dihydro-6H-purin-6-one | |
| 114 | 1-(4-chlorophenyl)-9-methyl-2-(4,4,4-trifluoro-3- | 385.01 |
| | methylbutyl)-1,9-dihydro-6H-purin-6-one | |
| 115 | 1-(4-chlorophenyl)-9-ethyl-2-(5,5,5-trifluoro-4- | 413.08 |
| | methylpentyl)-1,9-dihydro-6H-purin-6-one | |
| 116 | 1-(4-chlorophenyl)-9-methyl-2-(5,5,5-trifluoro-4- | 399.09 |
| | methylpentyl)-1,9-dihydro-6H-purin-6-one | |
| 117 | 9-ethyl-1-(4-fluorophenyl)-2-(4,4,4-trifluoro-3- | 383.11 |
| | methylbutyl)-1,9-dihydro-6H-purin-6-one | |
| 118 | 1-(4-fluorophenyl)-9-methyl-2-(4,4,4-trifluoro-3- | 369.10 |
| | methylbutyl)-1,9-dihydro-6H-purin-6-one | |
| 119 | 1-(4-chlorophenyl)-2-(2,6-difluorobenzyl)-9-(2,2,2- | 455.02 |
| | trifluoroethyl)-1,9-dihydro-6H-purin-6-one | |
| 120 | 1-(4-chlorophenyl)-2-(2,6-difluorobenzyl)-9-(2,2- | 437.03 |
| | difluoroethyl)-1,9-dihydro-6H-purin-6-one | |
| 121 | 1-(4-chlorophenyl)-9-(2,2,2-trifluoroethyl)-2-(4,4,4- | 453.05 |
| | trifluoro-2-methylbutyl)-1,9-dihydro-6H-purin-6- | |
| | one | |
| 122 | 1-(4-chlorophenyl)-9-(2,2-difluoroethyl)-2-(4,4,4- | 435.06 |
| | trifluoro-2-methylbutyl)-1,9-dihydro-6H-purin-6- | |
| | one | |
| 123 | 1-(4-chlorophenyl)-9-ethyl-2-(4-methylpentyl)-1,9- | 359.14 |
| | dihydro-6H-purin-6-one | |
| 124 | 9-ethyl-1-(4-fluorophenyl)-2-(5,5,5-trifluoro-4- | 397.13 |
| | methylpentyl)-1,9-dihydro-6H-purin-6-one | |
| 125 | 1-(4-fluorophenyl)-9-methyl-2-(5,5,5-trifluoro-4- | 383.12 |
| | methylpentyl)-1,9-dihydro-6H-purin-6-one | |
| 126 | 1-(4-chlorophenyl)-2-(2,2-dimethylpropyl)-9-ethyl- | 345.10 |
| | 1,9-dihydro-6H-purin-6-one | |
| 127 | 1-(4-chlorophenyl)-2-(2,2-dimethylpropyl)-9- | 331.11 |
| | methyl-1,9-dihydro-6H-purin-6-one | |
| 128 | 9-ethyl-1-(4-fluorophenyl)-2-(4-methylpentyl)-1,9- | 343.17 |
| | dihydro-6H-purin-6-one | |
| 129 | 1-(4-fluorophenyl)-9-methyl-2-(4-methylpentyl)- | 329.15 |
| | 1,9-dihydro-6H-purin-6-one | |
| 130 | 1-(4-chlorophenyl)-2-(2,2-dimethylbutyl)-9-ethyl- | 359.15 |
| | 1,9-dihydro-6H-purin-6-one | |
| 131 | 2-(2,6-difluorobenzyl)-9-ethyl-1-(4-fluorophenyl)- | 385.02 |
| | 1,9-dihydro-6H-purin-6-one | |
| 132 | 9-ethyl-1-(4-fluorophenyl)-2-(2,4,6- | 403.01 |
| | trifluorobenzyl)-1,9-dihydro-6H-purin-6-one | |
| 133 | 2-(2,4-difluorobenzyl)-9-ethyl-1-(4-fluorophenyl)- | 385.02 |
| | 1,9-dihydro-6H-purin-6-one | |
| 134 | 2-(2,5-difluorobenzyl)-9-ethyl-1-(4-fluorophenyl)- | 385.02 |
| | 1,9-dihydro-6H-purin-6-one | |
| 135 | 9-ethyl-1-(4-fluorophenyl)-2-[2- | 417.02 |
| | (trifluoromethyl)benzyl]-1,9-dihydro-6H-purin-6- | |
| | one | |
| 136 | 9-ethyl-2-(2-fluorobenzyl)-1-(4-fluorophenyl)-1,9- | 367.07 |
| | dihydro-6H-purin-6-one | |
| 137 | 2-(3,5-difluorobenzyl)-9-ethyl-1-(4-fluorophenyl)- | 385.04 |
| | 1,9-dihydro-6H-purin-6-one | |
| 138 | 2-(2-chlorobenzyl)-9-ethyl-1-(4-fluorophenyl)-1,9- | 383.01 |
| | dihydro-6H-purin-6-one | |
| 139 | 9-ethyl-1-(4-fluorophenyl)-2-[3- | 417.04 |
| | (trifluoromethyl)benzyl]-1,9-dihydro-6H-purin-6- | |
| | one | |
| 140 | 1-(4-chlorophenyl)-2-(2,4-difluorobenzyl)-9- | 386.98 |
| | methyl-1,9-dihydro-6H-purin-6-one | |
| 141 | 1-(4-chlorophenyl)-2-(2,5-difluorobenzyl)-9- | 386.98 |
| | methyl-1,9-dihydro-6H-purin-6-one | |
| 142 | 1-(4-chlorophenyl)-2-(2,3-difluorobenzyl)-9- | 386.99 |
| | methyl-1,9-dihydro-6H-purin-6-one | |
| 143 | 1-(4-chlorophenyl)-2-(2,6-difluorobenzyl)-9- | 386.98 |
| | methyl-1,9-dihydro-6H-purin-6-one | |
| 144 | 1-(4-chlorophenyl)-2-(3,5-difluorobenzyl)-9- | 386.98 |
| | methyl-1,9-dihydro-6H-purin-6-one | |
| 145 | 2-(2,3-difluorobenzyl)-9-ethyl-1-(4-fluorophenyl)- | 385.03 |
| | 1,9-dihydro-6H-purin-6-one | |
| 146 | 9-ethyl-1-(4-fluorophenyl)-2-{1-[3- | 431.04 |
| | (trifluoromethyl)phenyl]ethyl}-1,9-dihydro-6H- | |
| | purin-6-one | |
| 147 | 9-ethyl-1-(4-fluorophenyl)-2-[1-(2,4,6- | 417.01 |
| | trifluorophenyl)ethyl]-1,9-dihydro-6H-purin-6-one | |
| 148 | 1-(4-chlorophenyl)-2-(2-fluorobenzyl)-9-methyl- | 369.01 |
| | 1,9-dihydro-6H-purin-6-one | |
| 149 | 2-(2-chlorobenzyl)-1-(4-chlorophenyl)-9-methyl- | 384.97 |
| | 1,9-dihydro-6H-purin-6-one | |
| 150 | 2-(4-chloro-2-fluorobenzyl)-1-(4-chlorophenyl)-9- | 402.98 |
| | methyl-1,9-dihydro-6H-purin-6-one | |
| 151 | 2-(2-chloro-4-fluorobenzyl)-1-(4-chlorophenyl)-9- | 402.98 |
| | methyl-1,9-dihydro-6H-purin-6-one | |
| 152 | 2-(3-chloro-2-fluorobenzyl)-1-(4-chlorophenyl)-9- | 402.98 |
| | methyl-1,9-dihydro-6H-purin-6-one | |
| 153 | 9-ethyl-1-(4-fluorophenyl)-2-(2,4,5- | 403.02 |
| | trifluorobenzyl)-1,9-dihydro-6H-purin-6-one | |
| 154 | 2-(2-chloro-4-fluorobenzyl)-9-ethyl-1-(4- | 401 |
| | fluorophenyl)-1,9-dihydro-6H-purin-6-one | |
| 155 | 2-(3-chloro-2-fluorobenzyl)-9-ethyl-1-(4- | 401.02 |
| | fluorophenyl)-1,9-dihydro-6H-purin-6-one | |
| 156 | 2-(4-chloro-2-fluorobenzyl)-9-ethyl-1-(4- | 401.02 |
| | fluorophenyl)-1,9-dihydro-6H-purin-6-one | |
| 157 | 2-(2,4-dichlorobenzyl)-9-ethyl-1-(4-fluorophenyl)- | 416.98 |
| | 1,9-dihydro-6H-purin-6-one | |
| 158 | 2-(3,4-dichlorobenzyl)-9-ethyl-1-(4-fluorophenyl)- | 416.99 |
| | 1,9-dihydro-6H-purin-6-one | |
| 159 | 9-ethyl-1-(4-fluorophenyl)-2-(2,3,6- | 402.99 |
| | trifluorobenzyl)-1,9-dihydro-6H-purin-6-one | |
| 160 | 1-(4-chlorophenyl)-2-(2,4-dichlorobenzyl)-9- | 418.89 |
| | methyl-1,9-dihydro-6H-purin-6-one | |
| 161 | 1-(4-chlorophenyl)-2-(3,4-dichlorobenzyl)-9- | 418.89 |
| | methyl-1,9-dihydro-6H-purin-6-one | |
| 162 | 1-(4-chlorophenyl)-9-methyl-2-(2,3,6- | 404.94 |
| | trifluorobenzyl)-1,9-dihydro-6H-purin-6-one | |
| 163 | 2-(2-chloro-4-fluorobenzyl)-1-(4-fluorophenyl)-9- | 387.01 |
| | methyl-1,9-dihydro-6H-purin-6-one | |
| 164 | 2-(4-chloro-2-fluorobenzyl)-1-(4-fluorophenyl)-9- | 387.01 |
| | methyl-1,9-dihydro-6H-purin-6-one | |
| 165 | 2-(2,4-difluorobenzyl)-1-(4-fluorophenyl)-9-methyl- | 371.04 |
| | 1,9-dihydro-6H-purin-6-one | |
| 166 | 2-(2,3-difluorobenzyl)-1-(4-fluorophenyl)-9-methyl- | 371.03 |
| | 1,9-dihydro-6H-purin-6-one | |
| 167 | 2-(4-chloro-2-fluorobenzyl)-3-(4- | 404.86 |
| | chlorophenyl)thieno[3,2-d]pyrimidin-4(3H)-one | |
| 168 | 2-(2-chloro-6-fluorobenzyl)-1-(4-chlorophenyl)-9- | 402.91 |
| | methyl-1,9-dihydro-6H-purin-6-one | |
| 169 | 1-(4-chlorophenyl)-2-(2,6-dichlorobenzyl)-9- | 418.88 |
| | methyl-1,9-dihydro-6H-purin-6-one | |
| 170 | 1-(4-chlorophenyl)-9-ethyl-2-(5-fluoro-2,3-dihydro- | 409.03 |
| | 1H-inden-1-yl)-1,9-dihydro-6H-purin-6-one | |
| 171 | 2-(2-chloro-4-fluorobenzyl)-3-(4- | 404.89 |
| | chlorophenyl)thieno[3,2-d]pyrimidin-4(3H)-one | |
| 172 | 2-(4-chloro-2-fluorobenzyl)-3-(4-chlorophenyl)-7- | 418.89 |
| | methylthieno[3,2-d]pyrimidin-4(3H)-one | |
| 173 | 2-(4-chloro-2-fluorobenzyl)-1-(3,4-difluorophenyl)- | 404.93 |
| | 9-methyl-1,9-dihydro-6H-purin-6-one | |
| 174 | 2-(2-chloro-4-fluorobenzyl)-1-(3,4-difluorophenyl)- | 404.94 |
| | 9-methyl-1,9-dihydro-6H-purin-6-one | |
| 175 | 2-(2,4-dichlorobenzyl)-1-(3,4-difluorophenyl)-9- | 420.91 |
| | methyl-1,9-dihydro-6H-purin-6-one | |
| 176 | 4-[2-(2-chloro-4-fluorobenzyl)-9-methyl-6-oxo-6,9- | 411.99 |
| | dihydro-1H-purin-1-yl]benzamide | |
| 177 | 2-(2,6-dichlorobenzyl)-1-(4-fluorophenyl)-9- | 402.95 |
| | methyl-1,9-dihydro-6H-purin-6-one | |
| 178 | 2-(2,4-dichlorobenzyl)-1-(4-fluorophenyl)-9- | 402.94 |
| | methyl-1,9-dihydro-6H-purin-6-one | |
| 179 | 4-[2-(2-chloro-4-fluorobenzyl)-9-methyl-6-oxo-6,9- | 393.95 |
| | dihydro-1H-purin-1-yl]benzonitrile | |
| 180 | 2-(2,3-difluorobenzyl)-1-(3,4-difluorophenyl)-9- | 388.98 |
| | methyl-1,9-dihydro-6H-purin-6-one | |
| 181 | 2-(2,4-difluorobenzyl)-1-(3,4-difluorophenyl)-9- | 388.98 |
| | methyl-1,9-dihydro-6H-purin-6-one | |
| 182 | 1-(3,4-difluorophenyl)-9-methyl-2-(2,4,6- | 406.98 |
| | trifluorobenzyl)-1,9-dihydro-6H-purin-6-one | |
| 183 | 4-[2-(4-chloro-2-fluorobenzyl)-9-methyl-6-oxo-6,9- | 393.98 |
| | dihydro-1H-purin-1-yl]benzonitrile | |
| 184 | 4-[2-(2,4-difluorobenzyl)-9-methyl-6-oxo-6,9- | 378.03 |
| | dihydro-1H-purin-1-yl]benzonitrile | |
| 185 | 4-[2-(2,3-difluorobenzyl)-9-methyl-6-oxo-6,9- | 378.02 |
| | dihydro-1H-purin-1-yl]benzonitrile | |
| 186 | 1-(4-fluorophenyl)-9-methyl-2-(2,4,6- | 388.97 |
| | trifluorobenzyl)-1,9-dihydro-6H-purin-6-one | |
| 187 | 1-(4-fluorophenyl)-9-methyl-2-(2,3,6- | 390.05 |
| | frifluorobenzyl)-1,9-dihydro-6H-purin-6-one | |
| 188 | 2-(2-chloro-3,6-difluorobenzyl)-1-(4-fluorophenyl)- | 404.97 |
| | 9-methyl-1,9-dihydro-6H-purin-6-one | |
| 189 | 3-(4-chlorophenyl)-7-methyl-2-(2,4,6- | 420.89 |
| | trifluorobenzyl)thieno[3,2-d]pyrimidin-4(3H)-one | |
| 190 | 2-(2-chloro-3,6-difluorobenzyl)-1-(4-chlorophenyl)- | 420.95 |
| | 9-methyl-1,9-dihydro-6H-purin-6-one | |
| 191 | 2-(2-chloro-3,6-difluorobenzyl)-1-(3,4- | 422.97 |
| | difluorophenyl)-9-methyl-1,9-dihydro-6H-purin-6- | |
| | one | |
| 192 | 2-(2,3-dihydro-1H-inden-2-yl)-1-(4-fluorophenyl)- | 361.05 |
| | 9-methyl-1,9-dihydro-6H-purin-6-one | |
| 193 | 1-(4-chlorophenyl)-9-methyl-2-(2,3,4- | 404.91 |
| | trifluorobenzyl)-1,9-dihydro-6H-purin-6-one | |
| 194 | 1-(4-chlorophenyl)-2-(2,6-difluoro-3- | 400.94 |
| | methylbenzyl)-9-methyl-1,9-dihydro-6H-purin-6- | |
| | one | |
| 195 | 2-(3-chloro-2,6-difluorobenzyl)-1-(4-chlorophenyl)- | 420.88 |
| | 9-methyl-1,9-dihydro-6H-purin-6-one | |
| 196 | 2-(2,6-difluoro-3-methylbenzyl)-1-(4-fluorophenyl)- | 385 |
| | 9-methyl-1,9-dihydro-6H-purin-6-one | |
| 197 | 2-(3-chloro-2,6-difluorobenzyl)-1-(4-fluorophenyl)- | 404.95 |
| | 9-methyl-1,9-dihydro-6H-purin-6-one | |
| 198 | 4-[9-methyl-6-oxo-2-(2,4,6-trifluorobenzyl)-6,9- | 395.98 |
| | dihydro-1H-purin-1-yl]benzonitrile | |
| 199 | 1-(4-fluorophenyl)-2-isopropyl-9-methyl-1,9- | 286.99 |
| | dihydro-6H-purin-6-one | |
| 200 | 1-(4-chlorophenyl)-2-isopropyl-9-methyl-1,9- | 302.95 |
| | dihydro-6H-purin-6-one | |
| 201 | 1-(4-chlorophenyl)-9-ethyl-2-isopropyl-1,9-dihydro- | 317 |
| | 6H-purin-6-one | |
| 202 | 1-(4-fluorophenyl)-9-methyl-2-(2,3,4- | 388.97 |
| | trifluorobenzyl)-1,9-dihydro-6H-purin-6-one | |
| 203 | 1-(4-chloro-3-fluorophenyl)-2-(2,4-difluorobenzyl)- | 404.92 |
| | 9-methyl-1,9-dihydro-6H-purin-6-one | |
| 204 | 1-(4-chloro-3-fluorophenyl)-9-methyl-2-(2,4,6- | 422.92 |
| | trifluorobenzyl)-1,9-dihydro-6H-purin-6-one | |
| 205 | 1-(4-chloro-3-fluorophenyl)-9-methyl-2-(2,3,6- | 422.92 |
| | trifluorobenzyl)-1,9-dihydro-6H-purin-6-one | |
| 206 | 1-(4-chloro-3-fluorophenyl)-9-methyl-2-(2,3,4- | 422.91 |
| | trifluorobenzyl)-1,9-dihydro-6H-purin-6-one | |
| 207 | 2-(4-chloro-2-fluorobenzyl)-1-(4-chloro-3- | 420.9 |
| | fluorophenyl)-9-methyl-1,9-dihydro-6H-purin-6-one | |
| 208 | 2-(2-chloro-4-fluorobenzyl)-1-(4-chloro-3- | 420.88 |
| | fluorophenyl)-9-methyl-1,9-dihydro-6H-purin-6-one | |
| 209 | 1-(4-chloro-3-methylphenyl)-2-(2,4- | 400.92 |
| | difluorobenzyl)-9-methyl-1,9-dihydro-6H-purin-6- | |
| | one | |
| 210 | 2-(4-chloro-2-fluorobenzyl)-1-(4-chloro-3- | 416.9 |
| | methylphenyl)-9-methyl-1,9-dihydro-6H-purin-6- | |
| | one | |
| 211 | 2-(2-chloro-4-fluorobenzyl)-1-(4-chloro-3- | 416.98 |
| | methylphenyl)-9-methyl-1,9-dihydro-6H-purin-6- | |
| | one | |
| 212 | 1-(4-chloro-3-methylphenyl)-9-methyl-2-(2,4,6- | 419 |
| | trifluorobenzyl)-1,9-dihydro-6H-purin-6-one | |
| 213 | 1-(4-chloro-3-methylphenyl)-9-methyl-2-(2,3,4- | 418.89 |
| | trifluorobenzyl)-1,9-dihydro-6H-purin-6-one | |
| 214 | 1-(4-chloro-3-methylphenyl)-9-methyl-2-(2,3,6- | 418.91 |
| | trifluorobenzyl)-1,9-dihydro-6H-purin-6-one | |
| 215 | 3-(4-chlorophenyl)-7-methyl-2-(4,4,4- | 386.86 |
| | trifluorobutyl)thieno[3,2-d]pyrimidin-4(3H)-one | |
| 216 | 1-(4-chlorophenyl)-2-(2,3-dihydro-1H-inden-2-yl)- | 391.02 |
| | 9-ethyl-1,9-dihydro-6H-purin-6-one | |
| 217 | 1-(4-chlorophenyl)-2-(2,3-dihydro-1H-inden-2-yl)- | 376.92 |
| | 9-methyl-1,9-dihydro-6H-purin-6-one | |
| 218 | 1-(3,4-difluorophenyl)-2-(2,3-dihydro-1H-inden-2- | 378.94 |
| | yl)-9-methyl-1,9-dihydro-6H-purin-6-one | |
| 219 | 1-(4-fluorophenyl)-9-methyl-2-[5-(trifluoromethyl)- | 429.05 |
| | 2,3-dihydro-1H-inden-2-yl]-1,9-dihydro-6H-purin- | |
| | 6-one | |
| 220 | 2-[2-(1,3-benzothiazol-2-yl)ethyl]-1-(4- | 453.89 |
| | chlorophenyl)-9-ethyl-1,9-dihydro-6H-purin-6-one | |
| 221 | 2-[2-(1,3-benzothiazol-2-yl)ethyl]-1-(4- | 421.99 |
| | chlorophenyl)-9-methyl-1,9-dihydro-6H-purin-6- | |
| | one | |
| 222 | 1-(4-chlorophenyl)-9-methyl-2-[5-(trifluoromethyl)- | 445 |
| | 2,3-dihydro-1H-inden-2-yl]-1,9-dihydro-6H-purin- | |
| | 6-one | |
| 223 | 1-(4-chlorophenyl)-9-ethyl-2-[5-(trifluoromethyl)- | 459 |
| | 2,3-dihydro-1H-inden-2-yl]-1,9-dihydro-6H-purin- | |
| | 6-one | |
| 224 | 2-[2-(1,3-benzoxazol-2-yl)ethyl]-1-(4- | 419.89 |
| | chlorophenyl)-9-ethyl-1,9-dihydro-6H-purin-6-one | |
| 225 | 1-(4-chlorophenyl)-2-(3,4-dihydro-2H-chromen-4- | 406.9 |
| | yl)-9-ethyl-1,9-dihydro-6H-purin-6-one | |
| 226 | 2-[2-(1,3-benzoxazol-2-yl)ethyl]-1-(4- | 405.86 |
| | chlorophenyl)-9-methyl-1,9-dihydro-6H-purin-6- | |
| | one | |
| 227 | 1-(4-chlorophenyl)-2-[2-(dimethylamino)ethyl]-9- | 345.92 |
| | ethyl-1,9-dihydro-6H-purin-6-one | |
| 228 | 1-(4-chlorophenyl)-2-(3,4-dihydro-2H-chromen-4- | 392.94 |
| | yl)-9-methyl-1,9-dihydro-6H-purin-6-one | |
| 229 | 1-(4-chlorophenyl)-9-ethyl-2-vinyl-1,9-dihydro-6H- | 301.09 |
| | purin-6-one | |
| 230 | 1-(4-chlorophenyl)-2-(2,2-dimethylbutyl)-9-methyl- | 345.14 |
| | 1,9-dihydro-6H-purin-6-one | |
| 231 | 1-(4-chlorophenyl)-2-(3,3-dimethylbutyl)-9-ethyl- | 359.15 |
| | 1,9-dihydro-6H-purin-6-one | |
| 232 | 1-(4-chlorophenyl)-2-(3,3-dimethylbutyl)-9-methyl- | 345.13 |
| | 1,9-dihydro-6H-purin-6-one | |
| 233 | 1-(4-chlorophenyl)-9-(2,2-difluoroethyl)-2-(3,3,3- | 421.06 |
| | trifluoro-2-methylpropyl)-1,9-dihydro-6H-purin-6- | |
| | one | |
| 234 | 1-(4-chlorophenyl)-9-(2,2-difluoroethyl)-2-(4,4,4- | 421.05 |
| | trifluorobutyl)-1,9-dihydro-6H-purin-6-one | |
| 235 | 1-(4-chlorophenyl)-9-(2,2-difluoroethyl)-2-(4,4,4- | 435.06 |
| | trifluoro-3-methylbutyl)-1,9-dihydro-6H-purin-6- | |
| | one | |
| 236 | 1-(4-chlorophenyl)-9-(2,2-difluoroethyl)-2-(5,5,5- | 435.05 |
| | trifluoropentyl)-1,9-dihydro-6H-purin-6-one | |
| 237 | 1-(4-chlorophenyl)-9-(2,2,2-trifluoroethyl)-2-(3,3,3- | 439.15 |
| | trifluoro-2-methylpropyl)-1,9-dihydro-6H-purin-6- | |
| | one | |
| 238 | 1-(4-chlorophenyl)-9-(2,2,2-trifluoroethyl)-2-(4,4,4- | 453.18 |
| | trifluoro-3-methylbutyl)-1,9-dihydro-6H-purin-6- | |
| | one | |
| 239 | 1-(4-chlorophenyl)-9-(2,2,2-trifluoroethyl)-2-(5,5,5- | 453.18 |
| | trifluoropentyl)-1,9-dihydro-6H-purin-6-one | |
| 240 | 9-(2,2-difluoroethyl)-1-(4-fluorophenyl)-2-(4,4,4- | 419.06 |
| | trifluoro-2-methylbutyl)-1,9-dihydro-6H-purin-6- | |
| | one | |
| 241 | 9-(2,2-difluoroethyl)-1-(4-fluorophenyl)-2-(4,4,4- | 419.10 |
| | trifluoro-3-methylbutyl)-1,9-dihydro-6H-purin-6- | |
| | one | |
| 242 | 9-(2,2-difluoroethyl)-1-(4-fluorophenyl)-2-(5,5,5- | 419.10 |
| | trifluoropentyl)-1,9-dihydro-6H-purin-6-one | |
| 243 | 1-(4-chlorophenyl)-9-ethyl-2-(4-methylcyclohexyl)- | 371.15 |
| | 1,9-dihydro-6H-purin-6-one | |
| 244 | 1-(4-chlorophenyl)-9-ethyl-2-(4- | 399.15 |
| | isopropylcyclohexyl)-1,9-dihydro-6H-purin-6-one | |
| 245 | 2-(4-tert-butylcyclohexyl)-1-(4-chlorophenyl)-9- | 413.18 |
| | ethyl-1,9-dihydro-6H-purin-6-one | |
| 246 | 1-(4-chlorophenyl)-9-ethyl-2-[4- | 447.11 |
| | (trifluoromethyl)cyclohexyl]-1,9-dihydro-6H-purin- | |
| | 6-one | |
| 247 | 1-(4-chlorophenyl)-2-(4,4-difluorocyclohexyl)-9- | 393.10 |
| | ethyl-1,9-dihydro-6H-purin-6-one | |
| 248 | 9-ethyl-1-(4-fluorophenyl)-2-[4- | 409.13 |
| | (trifluoromethyl)cyclohexyl]-1,9-dihydro-6H-purin- | |
| | 6-one | |
| 249 | 1-(4-chlorophenyl)-9-methyl-2-[4- | 433.10 |
| | (trifluoromethyl)cyclohexyl]-1,9-dihydro-6H-purin- | |
| | 6-one | |
| 250 | 1-(4-fluorophenyl)-9-methyl-2-[4- | 395.13 |
| | (trifluoromethyl)cyclohexyl]-1,9-dihydro-6H-purin- | |
| | 6-one | |
| 251 | 4-[9-methyl-6-oxo-2-(4,4,4-trifluoro-3- | 376.12 |
| | methylbutyl)-6,9-dihydro-1H-purin-1- | |
| | yl]benzonitrile | |
| 252 | 4-[9-methyl-6-oxo-2-(4,4,4-trifluoro-2- | 376.13 |
| | methylbutyl)-6,9-dihydro-1H-purin-1- | |
| | yl]benzonitrile | |
| 253 | 2-(2-chloroethyl)-1-(4-chlorophenyl)-9-ethyl-1,9- | 337.05 |
| | dihydro-6H-purin-6-one | |
| 254 | 1-(4-chlorophenyl)-2-[2-(2,3-dihydro-1-benzofuran- | 421.11 |
| | 2-yl)ethyl]-9-ethyl-1,9-dihydro-6H-purin-6-one | |
| 255 | 1-(4-chlorophenyl)-2-[2-(2,3-dihydro-1-benzofuran- | 407.10 |
| | 2-yl)ethyl]-9-methyl-1,9-dihydro-6H-purin-6-one | |
| 256 | 2-[(E)-2-(1-benzofuran-2-yl)vinyl]-1-(4- | 403.07 |
| | chlorophenyl)-9-methyl-1,9-dihydro-6H-purin-6- | |
| | one | |
| 257 | 2-(1-benzothien-3-ylmethyl)-1-(4-chlorophenyl)-9- | 421.06 |
| | ethyl-1,9-dihydro-6H-purin-6-one | |
| 258 | 2-(1-benzothien-3-ylmethyl)-1-(4-chlorophenyl)-9- | 407.05 |
| | methyl-1,9-dihydro-6H-purin-6-one | |
| 259 | 1-(4-chlorophenyl)-2-(3-chloropropyl)-9-ethyl-1,9- | 351.07 |
| | dihydro-6H-purin-6-one | |
| 260 | 1-(4-chlorophenyl)-9-ethyl-2-[(3-methyl-1- | 449.05 |
| | benzothien-2-yl)methyl]-1,9-dihydro-6H-purin-6- | |
| | one | |
| 261 | 1-(3,4-difluorophenyl)-9-methyl-2-[(3-methyl-1- | 423.09 |
| | benzothien-2-yl)methyl]-1,9-dihydro-6H-purin-6- | |
| | one | |
| 262 | 9-ethyl-1-(4-fluorophenyl)-2-[5-(trifluoromethyl)- | 443.12 |
| | 2,3-dihydro-1H-inden-2-yl]-1,9-dihydro-6H-purin- | |
| | 6-one | |
| 263 | 1-(4-chlorophenyl)-2-(2,3-dihydro-1-benzofuran-3- | 407.11 |
| | ylmethyl)-9-ethyl-1,9-dihydro-6H-purin-6-one | |
| 264 | 1-(4-chlorophenyl)-2-(2,3-dihydro-1-benzofuran-3- | 393.10 |
| | ylmethyl)-9-methyl-1,9-dihydro-6H-purin-6-one | |
| 265 | 1-(3,4-difluorophenyl)-9-methyl-2-[5- | 447.09 |
| | (trifluoromethyl)-2,3-dihydro-1H-inden-2-yl]-1,9- | |
| | dihydro-6H-purin-6-one | |

The above exemplified compounds of the present invention have been tested in the following assay and generally possess an IC₅₀<300 nM and, in the majority of cases, <200 nM. Other assays, such as electrophysiology using rat VR1 expressed in HEK cells measuring activity at various pH levels, can be used.

### Determination of In Vitro Activity

In vitro activity of compounds was measured using one or both of the following assays.

### Method 1

CHO cells, stably expressing recombinant rat or human VR1 receptors and plated into black-sided 384 well plates, were washed three times with assay buffer (containing Hepes, NaCl₂, KCl, MgCl₂, CaCl₂, sucrose, glucose and probenecid, pH 7.4) and then incubated with test compound and 4 uM Fluo-3-AM for 60 minutes at room temperature in darkness. Cells were washed three times more to remove excess dye, before being placed, along with plates containing capsaicin and test compounds into a Molecular Devices FLIPR³⁸⁴. The FLIPR³⁸⁴ simultaneously performed automated pharmacological additions and recorded fluorescence emission from Fluo-3. In all experiments, basal fluorescence was recorded, before re-addition of test compounds and subsequent addition of a previously determined concentration of capsaicin that evoked 80% of the maximum response. Inhibition of capsaicin evoked increases in intracellular [Ca²⁺] were expressed relative to wells on the same plate to which an EC80 concentration of capsaicin was added in the absence of test compounds.

### Method 2

Antagonists were ranked by absolute efficacy at a single low concentration vs. activation by either pH 5.5 or capsaicin (500 nM) using a medium-throughput electrophysiology assay. TRPV1 activity is initially determined using a 5 second application of 500 nM capsaicin. Agonist (either pH 5.5 or capsaicin) is then applied for 5 seconds followed by a 30 second wash period until a stable control response is achieved. Inhibition of the agonist response is determined following applications of a single concentration of test compound and inhibition is monitored using repeated agonist activation in the presence of the compound until a stable inhibition state is achieved (up to a maximum of 10 minutes of application). If a successful recovery was achieved by re-applying a control wash, additional compounds can be tested sequentially. Inhibition effect of the drug is calculated as the sustained maximum current within the 5 second agonist application divided by the control sustained maximum current before the drug had been applied, multiplied by 100 (=% inhibition @ the test concentration).

1-15. (canceled)

16. A method of treating gout; irritable bowel syndrome; respiratory diseases such as chronic obstructive pulmonary diseases (COPD), chronic bronchitis, cystic fibrosis, asthma and rhinitis, including allergic rhinitis such as seasonal and perennial rhinitis, non-allergic rhinitis and cough; hot flushes; hiccups; obesity; or gastro-oesophageal reflux disease (GERD) comprising using a compound of formula (I): 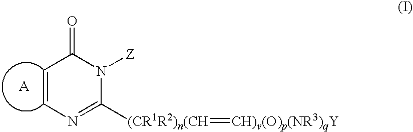
wherein:
A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by one, two or three groups independently chosen from halogen, hydroxy, S(O)ᵣC₁₋₄alkyl, S(O)ᵣNR⁴R⁵, —NR^{x}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a ring selected from phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
R¹ and R² are independently hydrogen, hydroxy, halogen, C₁₋₆alkyl or haloC₁₋₆alkyl, or R¹ and R² together form an oxo group;
R³ is hydrogen or C₁₋₆alkyl;
each R⁴ and R⁵ is independently hydrogen or C₁₋₆alkyl or R⁴ and R⁵, together with the nitrogen atom to which they are attached, may form a saturated 4-7 membered ring;
R^{x} is hydrogen or C₁₋₆alkyl;
n is zero, one, two, three or four;
v is zero or one;
p and q are both zero or one of p and q is zero and the other is one, provided that when n and v are zero then p and q are both zero;
r is zero, one or two;
Y is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S, the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;
Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by one or more groups independently chosen from halogen, hydroxy, cyano, nitro, NR⁴R⁵ as defined above, S(O)ᵣNR⁴R⁵, —NR^{x}S(O)ᵣC₁₋₆alkyl, S(O)ᵣC₁₋₄alkyl, C₁₋₆-alkyl, C₂₋₆alkenyl, C₂₋₆alkynyl, trifluoromethyl, C₁₋₆alkoxy, haloC₁₋₆alkoxy, C₃₋₇cycloalkyl and hydroxyC₁₋₆alkyl; or a pharmaceutically acceptable salt or tautomer thereof.

17. A method for the treatment of physiological disorders that may be ameliorated by modulating VR1 activity comprising using a compound of formula (I): 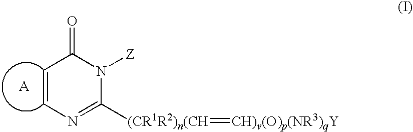
or a pharmaceutically acceptable salt or tautomer thereof wherein:
A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by one, two or three groups independently chosen from halogen, hydroxy, S(O)ᵣC₁₋₄alkyl, S(O)ᵣNR⁴R⁵, —NR^{x}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a ring selected from phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
R¹ and R² are independently hydrogen, hydroxy, halogen, C₁₋₆alkyl or haloC₁₋₆alkyl, or R¹ and R² together form an oxo group;
R³ is hydrogen or C₁₋₆alkyl;
each R⁴ and R⁵ is independently hydrogen or C₁₋₆alkyl or R⁴ and R⁵, together with the nitrogen atom to which they are attached, may form a saturated 4-7 membered ring;
R^{x} is hydrogen or C₁₋₆alkyl;
n is zero, one, two, three or four;
v is zero or one;
p and q are both zero or one of p and q is zero and the other is one, provided that when n and v are zero then p and q are both zero;
r is zero, one or two;
Y is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S, the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;
Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by one or more groups independently chosen from halogen, hydroxy, cyano, nitro, NR⁴R⁵ as defined above, S(O)ᵣNR⁴R⁵, —NR^{x}S(O)ᵣC₁₋₆alkyl, S(O)ᵣC₁₋₄alkyl, C₁₋₆alkyl, C₂₋₆alkenyl, C₂₋₆alkynyl, trifluoromethyl, C₁₋₆alkoxy, haloC₁₋₆alkoxy, C₃₋₇cycloalkyl and hydroxyC₁₋₆alkyl; wherein:
(a) when n is two and v is zero or when n is zero and v is one; and p and q are zero then A is a fused imidazole;
(b) when A is a fused 1,3-dimethyl[4,5]pyrazole ring and Z is optionally substituted phenyl then (CR¹R²)ₙ(CH═CH)ᵥ(O)ₚ(NR³)_{q}Y is not C₁₋₄alkyl, haloC₁₋₄alkyl, morpholinomethyl, piperidinomethyl, methoxymethyl, N-methylpiperazinomethyl or p-chlorophenoxymethyl;
(c) when A is a fused [3,4]thiophene ring then (CR¹R²)ₙ(CH═CH)ᵥ(O)ₚ(NR³)_{q}Y is not C₁₋₇alkyl; and
(d) when A is a fused [3,4]thiophene ring then (CR¹R²)ₙ(CH═CH)ᵥ(O)ₚ(NR³)_{q}Y¹ is not pyridylvinyl.

18. A compound of formula (IA): 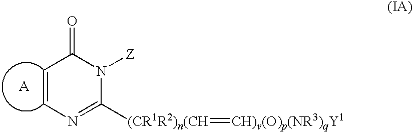
wherein:
A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by one, two or three groups independently chosen from halogen, hydroxy, S(O)ᵣC₁₋₄alkyl, S(O)ᵣNR⁴R⁵, —NR^{x}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl, or R¹ and R² together form an oxo group;
R¹ and R² are independently hydrogen, hydroxy, halogen, C₁₋₆alkyl or haloC₁₋₆alkyl, or R¹ and R² together form an oxo group;
R³ is hydrogen or C₁₋₆alkyl;
each R⁴ and R⁵ is independently hydrogen or C₁₋₆alkyl or R⁴ and R⁵, together with the nitrogen atom to which they are attached, may form a saturated 4-7 membered ring;
R^{x} is hydrogen or C₁₋₆alkyl;
n is zero, one, two, three or four;
v is zero or one;
p and q are both zero or one of p and q is zero and the other is one, provided that when n and v are zero then p and q are both zero;
r is zero, one or two;
Y¹ is C₁₋₆alkyl, C₂₋₆alkenyl, haloC₁₋₆alkyl, hydroxyC₁₋₆alkyl, aminoC₁₋₆alkyl, carboxyC₁₋₆alkyl; or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; a six-membered saturated ring containing one or two heteroatoms independently chosen from O and N; a 8 to 10-membered fused bicyclic partially saturated ring containing a C₅₋₆cycloalkyl ring, a five or a six-membered saturated ring containing one or two heteroatoms independently selected from O, N and S the ring fused to either a phenyl ring, a five-membered heteroaromatic ring as just defined or a six-membered heteroaromatic ring as just defined; or a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₆alkyl, C₂₋₆alkenyl, C₂₋₆alkynyl, nitro, cyano, C₃₋₇cycloalkyl, hydroxy, oxo, C₁₋₆alkoxy, haloC₁₋₆alkyl, phenyl, morpholino, haloC₁₋₆alkoxy, hydroxyC₁₋₆alkyl, hydroxyC₁₋₆alkoxy and NR⁴R⁵, wherein each of R⁴ and R⁵ are independently selected from hydrogen and C₁₋₆alkyl;
Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by one or more groups independently chosen from halogen, hydroxy, cyano, nitro, NR⁴R⁵ as defined above, S(O)ᵣNR⁴R⁵, —NR^{x}S(O)ᵣC₁₋₆alkyl, S(O)ᵣC₁₋₄alkyl, C₁₋₆alkyl, C₂₋₆alkenyl, C₂₋₆alkynyl, trifluoromethyl, C₁₋₆alkoxy, haloC₁₋₆alkoxy, C₃₋₇cycloalkyl and hydroxyC₁₋₆alkyl;
provided that:
(a) when n is two and v is zero or when n is zero and v is one; and p and q are zero then A is a fused imidazole;
(b) when A is a fused 1,3-dimethyl[4,5]pyrazole ring and Z is an optionally substituted phenyl then (CR¹R²)ₙ(CH═CH)ᵥ(O)ₚ(NR³)_{q}Y¹ is not C₁₋₄alkyl, haloC₁₋₄alkyl, morpholinomethyl, piperidinomethyl, methoxymethyl, N-methylpiperazinomethyl or p-chlorophenoxymethyl;
(c) when A is a fused [3,4]thiophene ring then (CR¹R²)ₙ(CH═CH)ᵥ(O)ₚ(NR³)_{q}Y¹ is not C₁₋₇alkyl;
(d) when A is a fused benzene ring or a fused [3,2]thiophene or [3,2]furan ring; n, p, q and v are zero; Z is an optionally substituted phenyl or optionally substituted pyrid-2-yl ring; and Y¹ is a phenyl, furan, thiophene or pyridine ring; then the Y¹ ring is not substituted by NR⁴R⁵;
(e) when A is a fused [2,3]thiophene ring, Z is optionally substituted phenyl, and p, q and v are zero then Y¹ is not C₁₋₆alkyl or an optionally substituted phenyl, or an optionally substituted 5 or 6 membered heteroaromatic ring or an optionally substituted six-membered saturated ring linked via an N heteroatom;
(f) when A is a fused [3,4]thiophene ring then (CR¹R²)ₙ(CH═CH)ᵥ(O)ₚ(NR³)_{q}Y¹ is not pyridylvinyl;
(g) when A is a fused [3,2]thiophene ring and Z is an optionally substituted phenyl then (CR¹R²)ₙ(CH═CH)ᵥ(O)ₚ(NR³)_{q}Y¹ is not methyl;
(h) when A is a fused benzene ring and Z is an optionally substituted phenyl then (CR¹R²)ₙ(CH═CH)ᵥ(O)ₚ(NR³)_{q}Y¹ is not phenyl or biphenyl; and
(i) when A is a fused [2,3]thiophene ring, n, v, p are zero, q is one and Z is an optionally substituted phenyl then Y¹ is not C₁₋₆alkyl;
or a pharmaceutically acceptable salt or tautomer thereof.

19. A compound according to claim 18 of formula (IAA): 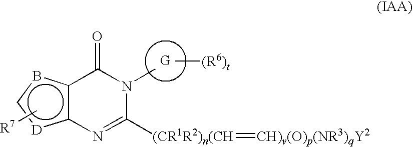
wherein:
B is S and D is C or one of B and D is N and the other N or S;
G is phenyl, pyridine or thiazole;
n is zero, one, two, three or four;
v is zero or one;
p and q are both zero or one of p and q is zero and the other is one, provided that when n and v are zero then p and q are both zero;
t is zero, one or two;
R¹ and R² are independently hydrogen, C₁₋₆alkyl or halogen;
R³ is hydrogen or C₁₋₆alkyl;
R⁶ is cyano, halogen, C₁₋₄alkyl, trifluoromethyl, C₁₋₄alkoxy, haloC₁₋₄alkoxy, amino, C₁₋₄alkylamino, di(C₁₋₆alkyl)amino, amide, C₁₋₄alkylamide or di(C₁₋₆alkyl)amide;
R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;
Y² is C₁₋₆alkyl, C₂₋₆alkenyl, haloC₁₋₆alkyl or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₄alkyl, hydroxy, oxo, C₁₋₄alkoxy, haloC₁₋₄alkyl, cyano, phenyl, haloC₁₋₄alkoxy, morpholino and NR⁴R⁵ where R⁴ and R⁵ are independently hydrogen or C₁₋₄alkyl or, R⁴ and R⁵, together with the nitrogen atom to which they are attached, form a 5 or 6 membered saturated ring;
provided that:
(a) when B is S and D is C; n, p, q and v are zero; G is phenyl or pyrid-2-yl ring; and Y² is a phenyl, furan, thiophene or pyridine ring; then the Y² ring is not substituted by NR⁴R⁵;
(b) when B is S and D is C and G is phenyl then (CR¹R²)ₙ(CH═CH)ᵥ(O)ₚ(NR³)_{q}Y² is not methyl;
(c) when n is two and v is zero or when n is zero and v is one; and p and q are zero then one of B and D is N and the other N or S;
or a pharmaceutically acceptable salt or tautomer thereof.

20. A compound according to claim 19 wherein one of B and D is N and the other N or S.

21. A compound according to claim 18 of formula (IIA): 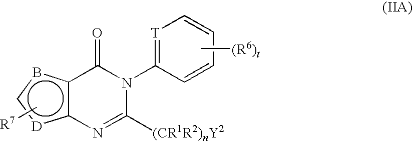
wherein:
one of B and D is N and the other N or S;
T is C or N;
n is zero, one, two, three or four;
t is zero, one or two;
R¹ and R² are independently hydrogen or C₁₋₆alkyl;
R⁶ is cyano, halogen, C₁₋₄alkyl, trifluoromethyl, C₁₋₄alkoxy, haloC₁₋₄alkoxy, amino, C₁₋₄alkylamino or di(C₁₋₆alkyl)amino;
R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;
Y² is C₁₋₆alkyl, haloC₁₋₆alkyl or a C₃₋₇cycloalkyl ring, a phenyl ring, a five-membered heteroaromatic ring containing one, two, three or four heteroatoms independently chosen from O, N and S, at most one heteroatom being O or S, a six-membered heteroaromatic ring containing one, two or three N atoms, an 8 to 10-membered fused bicyclic partially saturated ring containing a C₅₋₆cycloalkyl ring fused to either a phenyl ring, a five-membered heteroaromatic ring as just defined or a six-membered heteroaromatic ring as just defined, the ring being optionally substituted by one or more groups independently chosen from halogen, C₁₋₄alkyl, hydroxy, C₁₋₄alkoxy, haloC₁₋₄alkyl, phenyl, haloC₁₋₄alkoxy and NR⁴R⁵ where R⁴ and R⁵ are independently C₁₋₄alkyl or, R⁴ and R⁵, together with the nitrogen atom to which they are attached, form a 5 or 6 membered saturated ring;
or a pharmaceutically acceptable salt or tautomer thereof.

22. A compound according to claim 18 of formula (IB): 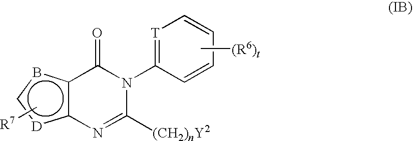
wherein:
one of B and D is N and the other N or S;
T is C or N;
n is zero, one, two, or three;
t is zero, one or two;
R⁶ is cyano, halogen, C₁₋₄alkyl, trifluoromethyl, C₁₋₄alkoxy, haloC₁₋₄alkoxy, amino, C₁₋₄alkylamino or di(C₁₋₆alkyl)amino;
R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;
Y² is C₁₋₆alkyl, C₂₋₆alkenyl, haloC₁₋₆alkyl or a C₃₋₇cycloalkyl ring; a phenyl ring; a benzoyl ring; a five-membered heteroaromatic ring containing one, two, three or four heteroatoms independently chosen from O, N and S, at most one heteroatom being O or S; a six-membered heteroaromatic ring containing one, two or three N atoms; an 8 to 10-membered fused bicyclic partially saturated ring containing a C₅₋₆cycloalkyl ring, a five or a six-membered saturated ring containing one or two heteroatoms independently selected from O, N and S the ring fused to either a phenyl ring, a five-membered heteroaromatic ring as just defined or a six-membered heteroaromatic ring as just defined; a six-membered saturated ring containing one or two heteroatoms independently selected from O, N and S; a 8 to 10 membered fused bicyclic heteroaromatic ring containing a phenyl ring, a five or six-membered heteroaromatic ring as just defined, the ring fused to either a five or six membered heteroaromatic ring as just defined; any of which rings being optionally substituted by one or more groups independently chosen from halogen, C₁₋₄alkyl, hydroxy, oxo, C₁₋₄alkoxy, haloC₁₋₄alkyl, cyano, phenyl, haloC₁₋₄alkoxy, morpholino and NR⁴R⁵ where R⁴ and R⁵ are independently hydrogen or C₁₋₄alkyl or, R⁴ and R⁵, together with the nitrogen atom to which they are attached, form a 5 or 6 membered saturated ring;
or a pharmaceutically acceptable salt or tautomer thereof.

23. A compound according to any one of claims 18 of formula (IIB): 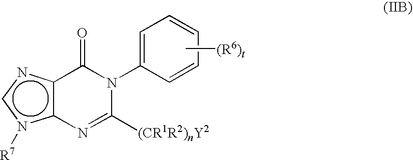
wherein n, t, R¹, R², R⁶, R⁷ and Y² are as defined in claim 4 or 6;
or a pharmaceutically acceptable salt or tautomer thereof.

24. A compound according to claim 18 of formula (IC): 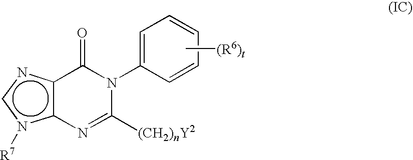
wherein n, t, R⁶, R⁷ and Y² are as previously defined;
or a pharmaceutically acceptable salt or tautomer thereof.

25. A compound according to claim 16 wherein Y² is C₁₋₆alkyl, haloC₁₋₆alkyl, C₂₋₆alkenyl or an optionally substituted ring selected from cyclopropyl, cylopentyl, cyclohexyl, phenyl, pyridinyl, thiazolyl, oxadiazolyl, tetrahydrobenzothiazolyl, benzoyl, tetrahydronaphthalenyl, oxazolyl, dihydrobenzofuranyl, benzofuranyl, tetrahydrothiopyranyl, dihydroindenyl, benzothiazolyl, dihydrochromenyl, benzoxazolyl, benzofuranyl and benzothienyl.

26. A pharmaceutical composition comprising one or more compounds of claims 18, or pharmaceutically acceptable salts thereof in association with a pharmaceutically acceptable carrier or excipient.

27. A process for the preparation of a compound of formula (I) of claim 16 comprising:
(A) reacting a compound of formula II: 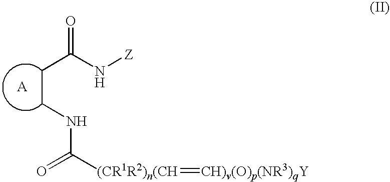
wherein n, p, q, v, A, R¹, R², R³, Y and Z are as defined in claim 1, with a cyclising agent;
(B) for compounds of formula (I) wherein n, p, q and v are zero and Y is an aromatic ring (Ar), reacting a compound of formula VIII with a compound of formula IX: 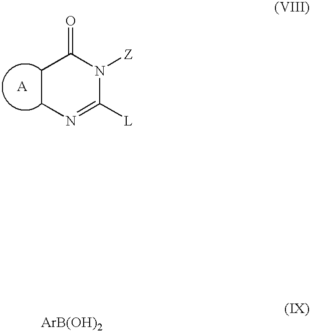
wherein A and Z are as defined in claim 1, Ar is an aromatic ring as defined for Y in claim 1 and L is a leaving group;
(C) for compounds of formula (I) wherein n is two and v is zero or n is zero and v is one; p and q are both zero; and R¹ and R² are both hydrogen, hydrogenation of a compound of formula XIV: 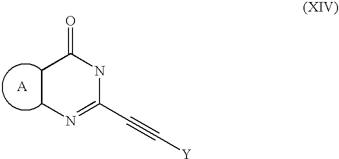
wherein A and Y are as defined in claim 1; or
(D) reacting a compound of formula XVI with a compound of formula VII: 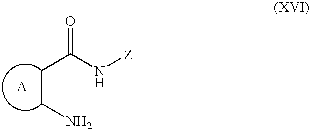
wherein A and Z are as defined in claim 16.
